(19) United States
(12) Patent Application Publication
(10) Pub. No.: US 2026/0306158 A1
(43) Pub. Date: Oct. 8, 2026

(54) FIBER-OPTICAL SENSOR SYSTEM FOR DATA TO FACILITATE, PLAN, LOCALIZE, MONITOR, OR CONFIRM ABLATION PROCEDURES

(71) Applicant: DEEPSIGHT TECHNOLOGY, INC., Santa Clara, CA (US)

(72) Inventors: Linhua XU, University City, MO (US); Jiangang ZHU, St. Louis, MO (US); Sitai KOU, Brentwood, MO (US); Lan YANG, Clayton, MO (US); Bradford WOOD, Bethesda, MD (US); Thomas HORNING, St Louis, MO (US); Maxwell ADOLPHSON, St Louis, MO (US)

(21) Appl. No.: 19/631,626

(22) Filed: Mar. 27, 2026

Related U.S. Application Data
(60) Provisional application No. 63/779,812, filed on Mar. 28, 2025.

(51) Int. Cl. A61B 18/00; G01D 5/353; G01D 21/02; G01K 11/32

(52) U.S. Cl. CPC A61B 18/00; G01D 5/35374; G01D 5/35387; G01D 21/02; G01K 11/32; A61B 2018/00577; A61B 2018/00648; A61B 2018/00702; A61B 2018/00791

(57) ABSTRACT

Disclosed are methods and devices for ablation, where an ablation system includes an ablation probe, an optical fiber extending along at least a portion of the ablation probe; a plurality of fiber Bragg grating (FBG) sensors disposed along the optical fiber and configured to generate temperature-related data; a fiber-end sensor disposed at a distal portion of the optical fiber and configured to sense acoustic signals; and one or more processors configured to: determine a temperature profile along the ablation probe based on the temperature-related data from the plurality of FBG sensors, and control at least one of positioning or operation of the fiber-end sensor based on the temperature profile.

### RELATED APPLICATIONS

This application claims the benefit of the filing date of U.S. Provisional Application No. 63/779,812, filed Mar. 28, 2025, the contents of which are incorporated by reference herein in their entirety.

This disclosure refers to U.S. Pat. No. 12,025,489, titled FIBER-OPTICAL SENSOR SYSTEM FOR ULTRASOUND SENSING AND IMAGING, issued on Jul. 2, 2024, to U.S. patent application Ser. No. 18/382,984, titled TRANSPONDER TRACKING AND ULTRASOUND IMAGE ENHANCEMENT and filed on Oct. 23, 2023 and published as U.S. Patent Pub. No. 2024/0423482, to patent application Ser. No. 18/609,378, titled FIBER-OPTICAL SENSOR ARRAY FOR SENSING AND IMAGING, filed on Mar. 19, 2024 and published as U.S. Patent Pub. No. 2024/0423481, to U.S. patent application Ser. No. 18/698,193, titled ULTRASOUND BEACON VISUALIZATION WITH OPTICAL SENSORS, filed on Oct. 7, 2022 and published as U.S. Patent Pub. No. 2025/02351800, to U.S. patent application Ser. No. 18/685,985, titled MULTI-DIMENSIONAL SIGNAL DETECTION WITH OPTICAL SENSORS, filed on Feb. 23, 2024 and published as U.S. Patent Pub. No. 2024/0358256, to U.S. patent application Ser. No. 18/597,493, filed on Mar. 6, 2024, titled Mixed Array Imaging Probe, and published as U.S. Patent Pub. No. 2024/0329243, to U.S. patent application Ser. No. 17/990,596, filed on Nov. 18, 2022, titled Mixed Ultrasound Transducer Arrays, and published as U.S. Patent Pub. No. 2023/0148869, and to U.S. patent application Ser. No. 17/244,605, filed on Apr. 29, 2021, titled Modularized Acoustic Probe, and published as U.S. Patent Pub. No. 2022/0350022, each of which is incorporated herein by reference in its entirety.

### FIELD

This following description relates generally to the field of fiber-optical based sensors. More specifically, this following description relates to the of fiber-optical based sensors used for planning, facilitating, monitoring, verifying, or augmenting ablation procedures.

### BACKGROUND

Ablation procedures may use various different modalities to achieve tissue ablation and may be conducted in various portions of the body. The various different ablation modalities, may include, for example, RF ablation, Cryoablation, laser ablation, microwave ablation, ultrasound ablation, such as high-intensity focused ultrasound (HIFU), histotripsy, vapor ablation, pulsed electrical fields, lithotripsy, and electroporation. Different ablation modalities may be delivered to patient tissue by different tools, including, for example, catheters, needles, robotic, surgical or laparoscopic, or endoscopic end effectors and other probes. Finally, the various ablation modalities and delivery tools may be employed in different portions of the body for destruction of tissue or modulation of biology or immune processes, or to augment drug delivery or therapeutic efficacy for example. It is advantageous to a clinician to monitor the tissue environment at and near the ablation site to gain insight into the progress of the ablation.

Ablation environments may be difficult to characterize or measure for several reasons. Depending on the ablation technology used, the tissue environment may be hostile to sensor technologies. For example, many ablation technologies generate extreme temperatures within the tissue, which can interfere with sensor. Some ablation technologies, e.g., radiofrequency or microwave ablation, saturate the tissue with high frequency electromagnetic waves, which may interfere with any sensors that employ electrically conductive components. Imaging tools such as ultrasound do not appreciate the ablation volume due to organic microbubbles and gas formation which results in artifacts and challenges in imaging the target region or the treated region.

Accordingly, there is a need for new and improved sensor technology to provide multimodal sensor measurements in hostile sensor environments, particularly for ablation procedure applications.

### SUMMARY

Systems, devices, and methods for facilitating ablation procedures are presented herein. In particular, systems, devices, and methods described herein may include fiber-optic based sensor devices and systems and methods of use. This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In some aspects, the techniques described herein relate to a method of manufacturing an ablation device, including forming an ablation probe having a proximal portion and a distal portion including a distal tip, forming a channel in an outer surface of the ablation probe, disposing at least one optical fiber of a fiber optic sensor device within the channel, positioning a sensing region of the optical fiber proximal to the distal tip of the ablation probe, and securing the optical fiber to the ablation probe, and or delivering multiple sensors on needles or the like in the expected ablation zone via a graded device such as a grid with regular spaced intervals.

In some aspects, the techniques described herein relate to a method of manufacturing an ablation device, including forming an ablation probe having a proximal portion and a distal portion including a distal tip, forming a lumen within the ablation probe, disposing at least one optical fiber of a fiber optic sensor device along the ablation probe, including at least partially within the lumen, or another location adjacent to the lumen or inside or outside of the lumen, with positioning a sensing region of the optical fiber proximal, inside or distal to the distal tip of the ablation probe, active treatment zone, and securing the optical fiber to the ablation probe, or affixing the fiber at regular intervals via independent insertion or a combined single device with grid insertion with parallel stabilization rods, for example.

In some aspects, the techniques described herein relate to an ablation system, including an ablation probe, a sensing system associated with the ablation probe and including at least one temperature or other sensor configured to generate temperature-related data, and at least one acoustic sensor configured to generate acoustic data, and one or more processors configured to determine a temperature profile based on the temperature-related data, and control at least one of operation, sensing, or positioning of the acoustic sensor based on the temperature profile. Imaging information or registered or fused information may also be fed into such a model or function as input for such a model in addition to the acousto optic sensor.

In some aspects, the techniques described herein relate to a method of operating an ablation system, including obtaining temperature-related data from a plurality of fiber Bragg grating (FBG) sensors disposed along an optical fiber, determining a temperature profile along an ablation probe based on the temperature-related data, obtaining acoustic data from a fiber-end sensor disposed at a distal portion of the optical fiber, and controlling at least one of positioning or operation of the fiber-end sensor based on the temperature profile or spatial profile of temperature and pressure information. This could help account for spatial heterogeneities and unpredictability of standard ablations which do not occur in uniform homogeneous fashion and are subject to conformal adjustments based on nearby anatomies such as convective heat loss from nearby blood flow. These conformal changes are not accounted for or predictable with standard ablation tools. Knowing the conformal nature of an ablation volume allows an operator to adjust the treatment to better treat tumor at risk for under treatment.

In some aspects, the techniques described herein relate to an ablation system, including an ablation probe, an optical fiber extending along at least a portion of the ablation probe, a plurality of fiber Bragg grating (FBG) sensors disposed along the optical fiber and configured to generate temperature-related data, a fiber-end sensor disposed at a distal portion of the optical fiber and configured to sense acoustic signals, and one or more processors configured to determine a temperature profile along the ablation probe based on the temperature-related data from the plurality of FBG sensors, and control at least one of positioning or operation of the fiber-end sensor based on the temperature profile.

In some aspects, there is provided an ablation system, including an ablation probe, a fiber optic sensing system including at least one fiber Bragg grating (FBG) sensor disposed on the ablation probe, the FBG sensor being configured to generate FBG sensor inputs including temperature-related data, and a fiber-end sensor disposed on a distal portion of the ablation probe, the FBG sensor being configured to generate fiber-end sensor inputs including acoustic data, and one or more processors configured to receive the FBG sensor inputs and the fiber-end sensor inputs, process the FBG sensor inputs and the fiber-end sensor inputs using a machine learning model, and generate, based on the processing, one or more outputs including at least one of a thermal prediction, an acoustic output, a zone boundary determination, or a control output for the ablation probe.

In some aspects, there is provided a method of operating an ablation system, including receiving FBG sensor inputs including temperature-related data from at least one fiber Bragg grating sensor, receiving fiber-end sensor inputs including acoustic data from a fiber-end sensor, processing the FBG sensor inputs and the fiber-end sensor inputs using a machine learning model, and generating, based on the processing, one or more outputs including at least one of a thermal prediction, an acoustic output, a zone boundary determination, or a control signal for controlling an ablation probe. In some embodiments, the FBG sensor inputs and the fiber-end sensor inputs may each include one or more data elements used to modify ablation planning, ablation guidance, monitoring, or verification software that might otherwise rely on simple geometries and therefore provide guidance that is not patient-specific, location-specific, or conformal. In some examples, the verification software may include one or more of Morpheus, Ablation Fit, See My Ablation Guide, NeuWave, Epione quantum surgical software, or Cascination software.

In some embodiments, the method may further include operating one or more needle-based sensors arranged for roughly parallel insertion relative to an ablation probe or treatment region. The one or more needle-based sensors may be independent or partly dependent, may share a common telescoping framework, or may buttress one another while still being configured to be actuated or advanced independently. Such an arrangement may permit sensor interrogation of a proximal ablation zone and/or a distal ablation zone located beyond an ablation volume, thereby improving monitoring, verification, and guidance of the ablation procedure.

Other features and aspects will be apparent from the following detailed description, the drawings, and the claims.

### BRIEF DESCRIPTION OF THE DRAWINGS

The accompanying figures, which are incorporated herein, form part of the specification and illustrate embodiments of systems, methods, and devices for ultrasound sensing and imaging. Together with the description, the figures further explain the principles of and enable a person skilled in the relevant art(s) to make and use the methods, systems, and devices described herein. The drawings are provided to illustrate various features of the embodiments described herein and are not necessarily drawn to scale. In the drawings, like reference numbers indicate identical or functionally similar elements.

FIG. 1 illustrates an optical sensor system for use with a fiber optic sensor device consistent with embodiments hereof.

FIG. 2 illustrates an optical sensor system for use with a fiber optic sensor device consistent with embodiments hereof.

FIG. 3 illustrates a sensor fiber including a fiber optic sensor device and associated optical fiber.

FIG. 4 illustrates a sensor fiber including an optical waveguide and optical resonator structure as an optical sensor.

FIGS. 5A and 5B illustrate examples of an optical sensor system and fiber optic sensor device consistent with embodiments hereof.

FIGS. 5C and 5D illustrate examples of an optical sensor system and fiber optic sensor device consistent with embodiments hereof.

FIGS. 6A-6G illustrate examples of an optical sensor system and fiber optic sensor device consistent with embodiments hereof.

FIGS. 6H and 6I illustrate examples of an optical sensor system and fiber optic sensor device consistent with embodiments hereof.

FIGS. 6J and 6K illustrate examples of an optical sensor system and fiber optic sensor device consistent with embodiments hereof.

FIG. 7A-7B illustrate an optical resonator structure including an in fiber Bragg grating consistent with embodiments hereof.

FIG. 8 illustrates a fiber optic sensor device including a membrane, consistent with embodiments hereof.

FIG. 9A-9C illustrate structural embodiments of fiber optic sensor devices consistent with embodiments hereof.

FIG. 10A-10C illustrate structural embodiments of fiber optic sensor devices consistent with embodiments hereof.

FIG. 11 illustrates an example optical sensing configuration for use with a fiber-end optical sensor device consistent with embodiments hereof.

FIG. 12 illustrates another example optical sensing configuration for use with fiber-optic sensor devices consistent with embodiments hereof.

FIG. 13 illustrates another example optical sensing configuration for use with fiber-optic sensor devices consistent with embodiments hereof.

FIG. 14A illustrates an example ablation device incorporating a fiber optic sensing configuration, consistent with embodiments hereof.

FIG. 14B illustrates an example ablation device incorporating a fiber optic sensing configuration, consistent with embodiments hereof.

FIG. 14C illustrates an example ablation device incorporating a fiber optic sensing configuration, consistent with embodiments hereof.

FIG. 14D illustrates an example ablation device incorporating multiple fiber optic sensor devices, consistent with embodiments hereof.

FIGS. 15A-15C illustrate example configurations for placement and integration of one or more fiber optic sensor devices with an ablation probe of an ablation device, consistent with embodiments hereof.

FIGS. 16A-16D illustrate example configurations of a fiber optic sensor device integrated with an ablation device, consistent with embodiments hereof.

FIGS. 17A and 17B illustrate example configurations of an ablation device integrated with a fiber optic sensor device, consistent with embodiments hereof.

FIGS. 18A and 18B illustrate example configurations of a fiber optic sensor device integrated with an ablation device, consistent with embodiments hereof.

FIG. 18C illustrates an example configuration in which an ablation probe is positioned relative to a target tissue region and an expected treatment volume.

FIG. 18D illustrates an example configuration in which a plurality of needle-based sensor delivery devices are arranged relative to the ablation probe and the expected treatment volume.

FIG. 18E illustrates an example needle-based sensor delivery device including a fiber-optic sensor and a plurality of sensing regions positioned along the fiber-optic sensor.

FIG. 18F illustrates an example processing arrangement in which outputs from the needle-based sensor delivery devices are provided to a processing system and one or more computational models.

FIG. 18G illustrates an example structured deployment device having a plurality of apertures configured to guide placement of needle-based sensor delivery devices.

FIG. 19 illustrates an example processing architecture for integrating sensing, prediction, imaging, and control operations of the ablation system, consistent with embodiments hereof.

FIG. 20 illustrates an example hardware architecture for implementing the machine learning model and processing operations described with respect to FIG. 19.

### DETAILED DESCRIPTION

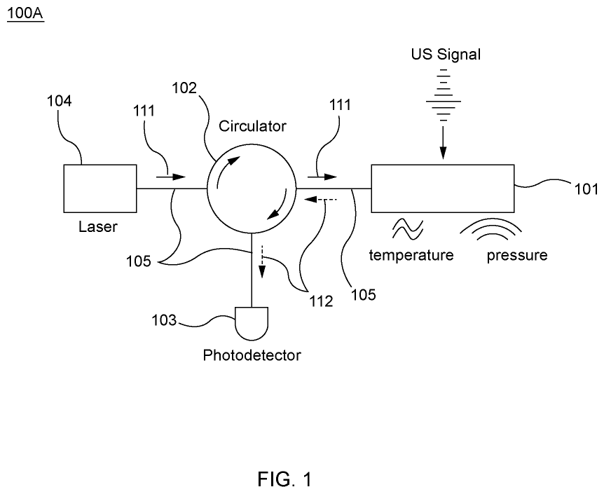

The present disclosure relates to systems, devices, and methods for facilitating ablation procedures using fiber-optic based sensing technologies that enable robust, multimodal measurement and imaging in sensor-hostile environments. In particular, an ablation probe is integrated with one or more optical fibers that include distributed sensing regions, such as fiber Bragg grating (FBG) sensors, and distal fiber-end sensors configured to detect acoustic signals. These optical sensors are capable of measuring environmental parameters, including temperature and pressure, as well as detecting acoustic signals for imaging, tracking, and guidance. By leveraging optical sensing modalities that are inherently immune to electromagnetic interference, the disclosed systems provide reliable operation in environments associated with RF, microwave, ultrasound, or other ablation modalities. As illustrated in FIGS. 1-18B, the fiber-optic sensor devices may be incorporated along the probe surface, within lumens, or at distal regions, enabling localized sensing and high-resolution mapping of tissue conditions proximate to the ablation site.

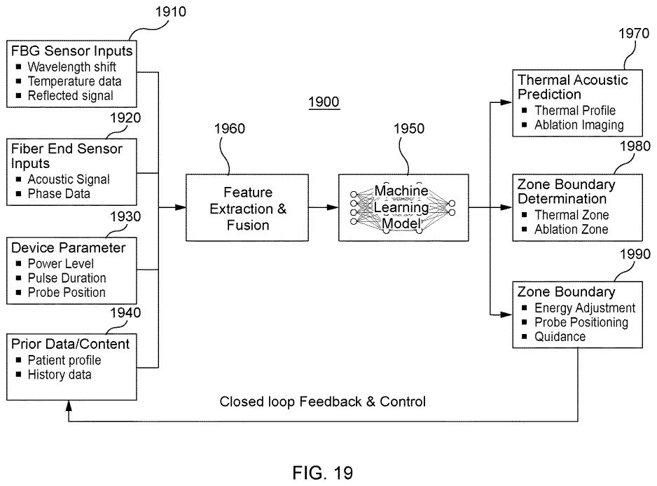

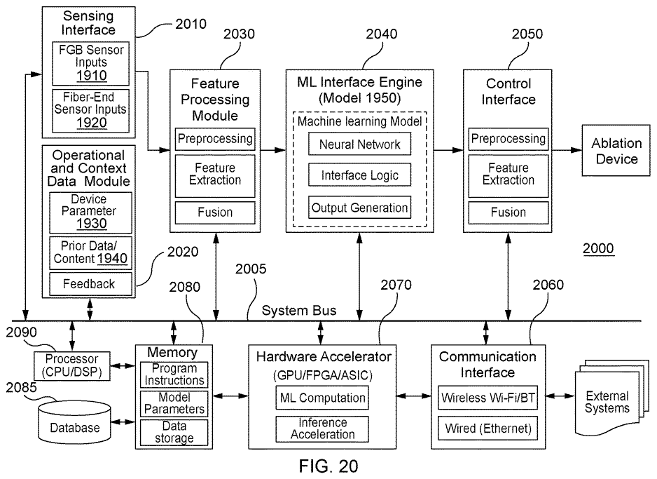

In further aspects, the disclosed systems incorporate advanced processing architectures that integrate multimodal sensor inputs to generate predictive, imaging, and control outputs for improved procedural efficacy. For example, as shown in FIGS. 19 and 20, temperature-related data from FBG sensors and acoustic data from fiber-end sensors may be processed using machine learning models to generate outputs such as thermal profiles, acoustic imaging, zone boundary determinations, and control signals for probe positioning and energy delivery. These models may perform feature extraction and fusion across sensing modalities to enable real-time or near-real-time feedback and feedback-driven control of the ablation procedure. Accordingly, the disclosed systems provide enhanced accuracy in monitoring ablation progression, delineating treatment zones, and guiding probe placement, thereby improving safety and treatment outcomes while reducing reliance on separate sensing and imaging systems.

It is advantageous to a clinician to monitor the tissue environment at and near the ablation site to gain insight into the progress of the ablation. Monitoring the ablation site may be performed via measurement and mapping of tissue temperature, measurement and mapping of tissue pressure, and via ultrasound imagery, or a combination thereof, including sensor input, or input over time or space, as one element feeding a multimodal model that determines outcomes, endpoints, and ablation confirmation via integration of dynamic temporal and spatial data feeding into a model that Integrates preprocedural imaging and post procedural imaging such as combining urgent assessment to determine the minimum ablation margin or tissue or tumor at risk for under treatment. Further, it is advantageous to provide imaging and tracking for the purposes of guiding an ablation delivery tool. This can be planning, navigation, localization, thermal feedback, monitoring ablation, determining tumor margin pretreatment, determining devascularization post treatment volume, and integration of spatial and temporal data to inform outcomes and procedural endpoints. Identification of ablation confirmation is highly correlated with successful outcomes in specific settings. Identification of tumor at risk for undertreatment via such temporal and spatial data should reduce risk for incomplete ablation or unsuccessful outcomes or regrowth or residual tumor.

Sensors deployed in specific locations on ultra small scales, such as 24 or 25 gauge needles, may provide a ray or vector of linear information over time and space with known spatial location and known temperature or pressure changes or tissue characterization changes such as are seen at tumor margin and ablation D vascularized margin. Such temporal and spatial information may also be integrated over a grid or other semi regulated fashion to provide input for models of ablation that incorporated pennes bioheat equation such as in a finite elemental analysis model or a deep learning model or other AI models described herein. Such sensors could be delivered near the periphery of an expected treatment volume to inform the exact location of the treatment volume or the D vascularized margin in comparison to pre-treatment imaging. Such confirmation of ablation or definition of minimum ablation margins may improve successful outcomes.

Non-limiting examples of various aspects and variations of the invention are described herein and illustrated in the accompanying drawings. The following detailed description is merely illustrative in nature and is not intended to limit the invention or the application and uses of the invention. Although the description of the invention is in the context of fiber optical sensor systems, methods, and devices for parameter measurement and acoustic signal detection, the disclosure should not be considered so limiting. For example, although methods may be discussed herein with respect to various medical procedures, embodiments hereof may be suitable for other medical procedures as well as other procedures or methods in other industries that may benefit from the sensing and imaging technologies described herein. Some of these embodiments might be enabled by the small scale of the fiber and the multimodal nature of the data and the location of the data at the “spatial point of care”, inside the body. Further, various systems and devices that incorporate fiber micro-sensors are described. It is understood that fiber micro-sensors, as described herein, may be integrated into and/or used with a variety of systems and devices not described herein. Modifications may be made to the embodiments described herein without departing from the spirit and scope of the present invention. Therefore, the following detailed description is not meant to be limiting. Furthermore, there is no intention to be bound by any expressed or implied theory presented in the preceding technical field, background, summary, or the following detailed description.

Various structures are described herein according to their geometric properties. As discussed herein, all structures so described may vary from the described shape according to the tolerances of known manufacturing techniques. Unless otherwise specified, features described with the term “substantially” are understood to be within 5% of exactness. For example, features described as “substantially parallel” may deviate from true parallel by 5%.

Systems, devices, and methods for performing sensor measurements during ablation procedures are provided. In particular, the sensor measurements are provided by fiber-optic based sensor devices. Fiber-optic based sensor devices consistent with the present disclosure may be employed for detection of environmental parameters (e.g., at least temperature sensing and/or pressure sensing) as well as for detection of acoustic signals (e.g., for tracking, guidance, object visualization/location, and/or ultrasound imaging). As used herein, environmental parameters refer to parameters that exist within a local vicinity of a fiber-optic based sensor device in use, for example, the temperature and pressure of tissue in the local vicinity of the sensor device. Further, when multiple environmental parameters are measured across an environment, e.g., temperature and/or pressure readings performed across an environment, environmental parameter mapping, e.g., of gradients and isobars or isotherms may be achieved. Although the systems, devices, and methods described herein are discussed with respect to their utility in ablation procedures, these may also be used be applied to other medical and surgical applications where similar functionality may be desired and/or advantageous.

The fiber-optic based sensor devices described herein incorporate optical devices disposed at the end of optical fibers or at designated locations along their length and configured for the detection of acoustic signals, including ultrasound signals. Fiber-optic based sensor devices, as described herein, may include an optical waveguide (such as an optical fiber) with one or more optical sensor devices either coupled to an end thereof or arranged along a length of the fiber. As used herein the term optical waveguide may refer to optical fibers, optical fiber cores, photonic integrated waveguides, planar waveguides, etc., based on material systems such as, for example, fused glass, polymer, semiconductor/dielectric wafer, nanoimprinted/3D printed polymer on different substrates or any other optical signal channel.

The technology described herein provides several advantages over conventional technologies, as described in greater detail below and throughout this disclosure. Fiber-optic based sensor devices described herein may have a smaller size than some conventional sensors. Smaller size sensors may be easier to incorporate as sensor arrays or with other surgical tools, i.e., as on-tool sensors. Ablation procedures may benefit from the increased location accuracy provided by the on-tool optical based ultrasound transducers described herein to ensure that ablation tools are appropriately located within an ablation target. In addition to the increased location accuracy, real-time or near-real time tracking or location monitoring may be beneficial to ensure that the ablation tool is not displaced from the target location during a procedure.

Fiber-optic based sensor devices described herein may provide robust measurement stability within the sensor-hostile environments associated with different ablation techniques. Fiber-optic based sensor devices may be provided with optical fibers that are non-conductive of RF and microwave energy. Thus, the RF and/or microwave signals generated for ablation procedures do not interfere with the optical signals used in the optical based measurement sensors. Additionally, as described herein, techniques may be used to account for temperature variations of the fiber-optic based sensor devices. In an ablation environment, both the target tissue and the ablation tool may undergo significant heating or cooling. Such heating or cooling may distort results from some other types of sensors used for measurement purposes during an ablation procedure. Given the localization capabilities of the optical sensor, multiple substantially parallel sensor fibers could be placed at the expected periphery of an ablation zone where temperatures are not as damaging to any sensor. The successful use and placement of the acousto-optic sensor(s) in this peripheral location is based on its localization capabilities as well as its thermal and pressure capabilities. The localization of margins is critical determinant of success and failure of a focal ablation such as in the liver or kidney.

In a further advantage, the fiber-optic based sensor devices may be used multi-modally, to measure multiple different parameters. The same sensors may be employed to measure temperature, tissue pressure, and may be used to provide ultrasound based guidance, tracking, location, and imaging. Such temperature and/or pressure measurements may be used to determine the extent of the ablation effect, e.g., based on tissue temperature or tissue pressure. Such temperature and pressure measurement may also be used to track and identify the boundaries of tissue isotherms created during an ablation procedure. Temporal and spatial localization of isotherms may determine thermal effects on tissue, minimum ablation margins, and or immunomodulatory effects both localized and systemic, knowing the temporal and spatial factors and history related to a specific voxel of tumor may enable A favorable immunomodulatory effect by enhancement of tumor specific immune stimulation such as recruitment of T cells, antigen presenting cells, appropriate macrophage polarized cells, and potentially a reversal of regulatory T cells all of which may promote a positive immune response or subsequent creation of terminal matrices within tumor associated lymphoid cellular clusters or miniature lymph nodes in the tissue surrounding an ablation, highly associated with positive immune effects for Tumor ablation combined with immunomodulators such as checkpoint inhibitors. Temperature measurement via optical sensors may have advantages over more traditional temperature transducers for several reasons. First, by using the optical sensors for temperature measurement, it may be unnecessary to provide additional temperature sensors and wiring which would lead to a bulkier ablation tool or measurement probe. Next, as discussed above, optical based sensors are robust against interference from the RF and microwave energy generated during an ablation procedure. The ability to use the fiber-optic based sensor devices described herein to provide multimodal measurements may simplify tool and probe design. Small scale sensors may also be used in multiplicity without increased risk which is quite small for 24 or 25 gauge needles but much greater for larger probes as is typical for other thermal techniques.

Fiber-optic based sensor devices provided herein may be used to provide temperature measurements. Such sensor devices may be provided as on-tool sensors and/or via separate probes. Multiple temperature sensors may be provided so as to permit temperature mapping, e.g., based on temperature determinations recorded in different locations.

Fiber-optic based sensor devices provided herein may be used to provide tissue pressure measurements. Such sensor devices may be provided as on-tool sensors and/or via separate probes. Multiple pressure sensors may be provided so as to permit pressure mapping, e.g., based on pressure determinations recorded in different locations.

Fiber-optic based sensor devices provided herein may be used to provide ultrasound based object visualization, tissue visualization, tracking, guidance, and/or location determination. Object/tissue visualization, tracking, guidance and location determination are important aspects for performing ablation procedures in a safe and reliable manner. Objects for tracking, visualization, and location determination may include any type of ablation device that travels or is located within the body of a subject, including, for example, catheters, needles, and other ablation devices. For instance, medical practitioners may visualize and track an ablation probe or tip while performing an ablation to ensure safety and efficacy. In such instances, accurate ablation probe visualization or tracking may help to guide the ablation probe to the appropriate ablation location as well as to prevent or reduce unintentional vascular, neural, tissue or visceral injury.

The optical signal changes induced by the acoustic waves, temperature variation, and tissue pressure changes occur on different time scales and can be separated with appropriate signal processing techniques, such as time-frequency analysis, filtering, or machine learning algorithms, allowing independent extraction of both dynamic acoustic responses and slower thermal and/or tissue pressure effects for enhanced sensing precision.

Fiber optic sensor devices consistent with the present disclosure may include resonant structures, including, but not limited to Fabry-Perot (FP) resonators, optical cavity resonators, whispering-gallery-mode resonators, and photonic crystal resonators; optical interferometers, including but not limited to MZI, phase-shift coherent interferometers, self-mixing interferometers; polarization based sensors; fiber Bragg grating based sensors; and acoustically responsive fiber end facets.

The aforementioned fiber optic sensor devices are configured to respond to and/or detect temperature, pressure, and acoustic (such as ultrasound) signals. These optical structures may include temperature sensitive, pressure sensitive, and acoustically responsive materials and/or acoustically responsive structures.

Acoustically responsive, as used herein, refers to structures or materials that are configured to respond to incident acoustic signals (e.g., ultrasound acoustic signals) in a manner that adjusts the optical properties of the materials or structures. Reponses to acoustic signals in such resonant, interferometer, polarization based, acoustically responsive fiber end facet, and/or any other structures described herein may be due to the photo-elastic effect and/or physical deformation of the structures. When subject to acoustic signals, the resonant, interferometer or acoustically responsive fiber end facet structures are subject to mechanical stress and/or strain from the alternating pressures of the acoustic signal sound waves. This mechanical stress and/or strain may change the optical properties of the optical sensor structures due to the photo-elastic effect and may also cause changes or deformations in the physical structure of resonator. With polarization-based sensors, the polarization of optical signals changes when the medium through which the light is passing is subjected to acoustic signals. When coupled to a light source (e.g. a laser light source, a broadband light source (e.g. a lamp or LED) or other suitable light source) via an optical waveguide (e.g., an optical fiber), the effect of acoustic signals on the optical sensor structures may be measured due to changes in the light returned by the optical sensor structures via the optical waveguide.

Temperature sensitive, as used herein, refers to structures and materials configured to change properties in accordance with temperature changes. Property changes responsive to temperature changes may occur at the material level and/or at the structural level of the devices described herein. The property changes may be detected through the use of coherent light directed through the optical waveguides to which the sensors are coupled. Interaction between the sensor and the light may be altered according to the temperature of the fiber optic sensor device, thus permitting measurement of temperature in or near the fiber optic sensor device. In some embodiments, acoustic signals may be employed to assist in temperature measurements. The level of acoustic response provided by a structure or material may be affected by the temperature, and thus, in some embodiments, temperature may be measured during provision of an acoustic signal.

Pressure sensitive, as used herein, refers to structures and materials configured to change properties in accordance with pressure changes. Property changes responsive to pressure changes may occur at the material level and/or at the structural level of the devices described herein. The property changes may be detected through the use of coherent light directed through the optical waveguides to which the sensors are coupled. Interaction between the sensor and the light may be altered according to the pressure of tissue surrounding the fiber optic sensor device, thus permitting measurement of pressure in or near the fiber optic sensor device. In some embodiments, acoustic signals may be employed to assist in pressure measurements. The level of acoustic response provided by a structure or material may be affected by the baseline pressure to which the structure or material is subject, and thus, in some embodiments, temperature may be measured during provision of an acoustic signal.

Within this disclosure, optical signals and light may be referred to as responding to temperature, pressure, and/or acoustic signals. It is understood that such responses are due to interaction between the acoustic signals and the medium through which the light passes and/or due to pressure or temperature induced property changes. Thus, as discussed herein, a material or structure that is referred to as temperature sensitive, pressure sensitive, or acoustically responsive may respond to environmental parameters such as temperature and pressure and to acoustic signals typical of an ultrasound environment in manner that can be measured, by techniques discussed herein, by optical signals consistent with embodiments hereof.

Embodiments hereof include systems configured for use with fiber optic sensor devices. For example, systems consistent with the present disclosure may include light sources (e.g., laser light sources, a broadband light source (e.g. a lamp or LED), an optical system on a chip, or other suitable light source), light reception devices (e.g., photodetectors, etc.), optical devices (splitters, combiners, circulators, polarization sensitive couplers, polarization analyzers, polarization controllers, frequency shifters, etc.), control devices, computer processing units, and other devices to facilitate the functionality of the fiber optic sensor devices. Further, such systems consistent with the present disclosure may include acoustic devices, such as transducers, probes, and hardware/software for their control. Systems consistent with the present disclosure may further include medical systems and devices, including all devices, systems, hardware, and software necessary to carry out any medical procedures that the fiber optic sensor devices are used to facilitate.

FIG. 1 illustrates an optical sensor system for use with a fiber optic sensor device. The optical sensor system 100A includes a light source 104, such as a laser, a light reception device 103, such as a photodetector, one or more optical waveguides 105, an optical circulator 102, and a fiber optic sensor device 101. In operation, the light source 104 supplies the initial optical signal 111 to the fiber optic sensor device 101 via the optical waveguides 105 and through the optical circulator 102. The supplied initial optical signal 111 is returned by the fiber optic sensor device 101 back along the optical waveguide 105. The returned optical signal 112 travels via the optical waveguides 105 through the optical circulator 102 and is received at the light reception device 103. As discussed above, environmental temperature, environmental pressure, and/or acoustic signals incident on the fiber optic sensor device 101 alter the optical characteristics (which may include the physical structure as well as the optical material properties) of the fiber optic sensor device 101. Such optical characteristic alterations may be measured according to changes in the returned optical signal 112, as discussed in greater detail below, thus permitting pressure and temperature measurement as well as acoustic based measurements for location, guidance, tracking, and imaging. This can be accomplished at a single point in time and space for a sensor or at multiple points along a vector on a needle with multiple sensors or on a needle that is pulled back with single or multiple sensors for multiple time and space data points, all encoded with localized information.

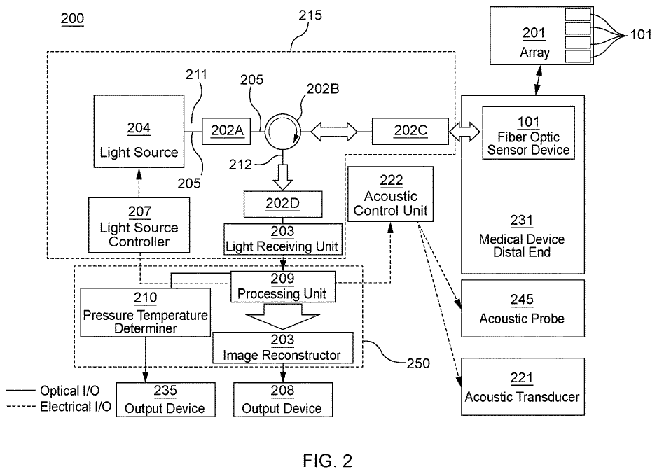

FIG. 2 illustrates a fiber optic sensor system for use with a fiber optic sensor device. The fiber optic sensor system 200 includes components, devices, hardware, and software to facilitate the use of a fiber optic sensor device 101 or fiber optic sensor device array 201 (comprising a plurality of fiber optic sensor devices 101, as pictured in FIG. 2, or a series of sensors along the length of the optical fiber waveguide). Further references to FIG. 2 may refer specifically to the use of a single fiber optic sensor device 101; however, it will be understood that, in additional embodiments, a fiber optic sensor device array 201 may be incorporated into the fiber optic sensor system 200 in combination with any of the features discussed below and that any functionality attributed to a fiber optic sensor device 101 may further be carried out by the fiber optic sensor device array 201. In embodiments, for example as shown in FIG. 2, the fiber optic sensor system 200 may include hardware and componentry to facilitate the use of additional medical or surgical tools, including, for example, an ultrasound transducer and/or ultrasound probe, as well as ablation devices. The ultrasound transducer may be used for generating and receiving acoustic signals or simply generating acoustic signals. The fiber optic sensor system 200 may include one or more processors or a processing system 250, an optical sub-system 215, and an output device 208.

The processing system 250 may include a processing unit or processor 209, an image reconstruction unit or image reconstructor 206, and a pressure/temperature determination unit or a pressure/temperature determiner 210. Processing unit 209 may include at least one computer processor, at least one non-transitory computer readable storage medium, and appropriate software instructions. The processing unit 209 is configured to provide control signals to and receive information signals from the light source control unit 207, the light receiving device 203, and the acoustic control unit 222. The processing unit 209 may communicate (via control signals and information signals) with the light source control unit 207, thereby providing control of optical signals provided to the fiber optic sensor device 101. The processing unit 209 may communicate (via control signals and information signals) with the acoustic control unit 222, thereby providing control and reception of acoustic signals via an acoustic probe 245. The processing unit 209 is further configured to communicate with the light receiving device 203 to receive information signals associated with optical signals received by the light receiving device 203. Thus, processing unit 209 operates to provide the necessary control signals and receive the acquired information signals in the fiber optic sensor system 200.

The processing unit 209 is further in communication with the image reconstruction unit 206, which operates to generate images based on the data and/or information acquired by the processing unit 209. The image reconstruction unit 206 may generate images based on data related to a medium, such as a human body, captured by the fiber optic sensor device 101 and/or the acoustic probe 245. The image reconstruction unit 206 may be integrated within the same hardware containing the processing unit 209 and/or may be a separate system including at least one computer processor, at least one non-transitory computer readable storage medium, and appropriate software instructions.

The processing unit 209 is further in communication with the pressure/temperature determination unit 210, which operates to determine environmental parameters such as temperature and pressure based on the data and/or information acquired by the processing unit 209. The pressure/temperature determination unit 210 may determine temperature and pressure data (including discrete data points as well as temperature or pressure maps) related to a medium or environment, such as a human body, captured by the fiber optic sensor device 101. The pressure/temperature determination unit 210 may be integrated within the same hardware containing the processing unit 209 and/or may be a separate system including at least one computer processor, at least one non-transitory computer readable storage medium, and appropriate software instructions. In embodiments where a single fiber is used for sensing pressure and/or temperature as well as incident acoustic signals, the pressure/temperature determination unit 210 may use various techniques and methods to differentiate optical signals representative of incident acoustic signals waves from signals representative of pressure and/or temperature. As discussed above, the optical signal changes induced by incident acoustic signals, temperature variation, and tissue pressure changes occur on different time scales and may be separated with appropriate signal processing techniques, such as time-frequency analysis, filtering, or machine learning algorithms, allowing independent extraction of both dynamic acoustic responses and slower thermal and/or tissue pressure effects for enhanced sensing precision. In further embodiments, acoustic signals may be switched on and off, permitting the measurement of pressure and/or temperature during a period where there are no incident acoustic signals.

The processing system 250 may provide control signals to an output device 208 to provide a data output. The output device 208 may include, for example, a display or a device including a display.

The medical device distal end 231 may include one or more of a needle, a catheter, a guidewire, a delivery device, and/or any other device or apparatus configured for use within the body of a patient. The medical device distal end 231 may be configured for the performance of an ablation procedure.

In some embodiments, the output device 208 may further include an additional systems, such as a medical procedure system that is configured to use the data that is output. For example, output device 208 may include an ablation system or any other appropriate medical system, including an endoscopy system, a laparoscopic system, a robotic surgical system, neurosurgical system and an interoperative ultrasound imaging system.

The output data may include information related to guidance, location, tracking, and imagery of the medical device distal end 231 or working portion, images acquired of the medium in the area of where the medical device distal end 231 is used/deployed such as the patient anatomy, as well as temperature and pressure information of the local tissue environment.

The optical sub-system 215 includes a light source control unit 207, a light source 204, optical devices 202A, 202B, 202C, and 202D, and light receiving device 203. The light source control unit is configured to interface with and control the light source 204 to control the production of an initial optical signal 211. The light source may generate a continuous wave (CW) or pulsed light emission (stimulated emission, spontaneous emission, and/or the like.) The initial optical signal 211 may include coherent light, e.g. laser light, provided in one or more modes and at one or more frequencies. The initial optical signal 211 may be of a single frequency/wavelength, a selection of frequencies/wavelengths, and/or a broadband light source. Thus, light source 204 may include a laser array configured to produce laser light in one or more modes and at one or more frequencies. Additionally, the polarization of the supplied light may be controlled to optimize the detected signal levels according to application requirement. The polarization state of light can be controlled to be linear polarized at certain angles or to be circularly polarized. Linearly polarized light will respond optimally to a certain input ultrasound direction, and circularly polarized light will respond to ultrasound from all directions. The polarization of light can be defined from the laser source output, and the output polarization state can be controlled by an in-line fiber polarizer, a paddle fiber polarization controller, an in-line fiber polarization controller, or other types of polarization controller. The optical devices 202A, 202B, and 202C may be configured to manipulate or influence the initial optical signal 211 received at the fiber optic sensor device 101. The initial optical signal 211 may be provided at a plurality of wavelengths or across a spectrum of wavelengths. The optical device 202A may include, for example, a wavelength division multiplexing (WDM) device configured to multiplex multiple frequencies of initial optical signal 211 provided by the light source 204 for simultaneous transmission over the optical waveguides 205 that direct the initial optical signal 211 to the fiber end optical sensor 101. The optical device 202B may be a circulator with first, second and third ports, where the first port is in optical communication with the light source through a wavelength division multiplexing device (WDM) 202A. The initial optical signal 211 may pass through a second optical device 202B, which may be an optical circulator, for example, and which is configured to direct the initial optical signal 211 to the optical device 202C. The optical device 202C may include a WDM device configured to de-multiplex the initial optical signal 211 provided to the fiber optic sensor device 101, which may be part of an array 201 such that each of multiple fiber optic sensor devices 101 receives and subsequently outputs light of a different wavelength. Optical device 202C is in optical communication with the second port of the second optical device 202B for dividing the initial optical signal into optical signals each having one of the wavelengths associated therewith and combining the returned optical signals from the fiber optic sensor device 101 which is then directed though a third port and optical device 202D which may include a WDM device, to the light receiving device 203.

The initial optical signal 211 is received by the fiber optic sensor device 101 (or optical sensors 101 of the fiber optic sensor device array 201 some embodiments) and returned through one or more optical waveguides 205 to the optical device 202C, which may be further configured to multiplex the returned optical signal 212 (if required) for transmission to the light receiving device 203. The returned optical signal 212 may be directed by the optical device 202C through the optical device 202B and towards the optical device 202D, which may be a WDM device configured to de-multiplex the returned optical signal 212 for reception by the light receiving device 203.

Optical device 202D may be in optical communication with the third port of the optical device 202B for receiving the returned optical signal and dividing it into individual wavelength components. The light receiving device 203, which may be a photodetector array, for example, may be in optical communication with optical device 202D for receiving the individual wavelength components of the returned optical signal, such that detected phase shifts or other changes in the individual wavelength components are indicative of sensed acoustic signals.

It will be understood that, in embodiments that do not require frequency multiplexing/demultiplexing of the initial optical signal 211 and the returned optical signal 212, the optical devices 202A and 202C may not be required. The light receiving device 203 may include any suitable device configured to detect incident light, including, for example, a photodetector. The light receiving device 203 may further include, but is not limited to, a photodiode. The light receiving device 203 may be in optical communication with the optical device 202D (e.g., a wavelength division multiplexing splitter) for receiving the individual wavelength components of the returned optical signal 212, such that detected phase shifts, changes in polarization, or other changes in the individual wavelength components are indicative of sensed acoustic signals. The changes in the returned optical signal 212 may be converted (e.g., by the processing unit 209 and/or by additional optical components such as polarization sensitive couplers and/or frequency shifters) into data representative of temperature, pressure, and/or sensed acoustic signals (which may be further used, e.g., to generate data representative of the tissue/anatomical structure of the medium in which the medical device distal end 231 is inserted in the area of a diagnostic or a therapeutic procedure and/or to identify a location of the medical device distal end 231 within the medium and/or to assist in the aforementioned temperature and pressure measurements).

In embodiments, the initial optical signal 211 and returned optical signal 212 signals may undergo pre-processing, beamforming and post-processing, as described in U.S. patent application Ser. No. 18/698,193, filed Apr. 3, 2024, titled Ultrasound Beacon Visualization with Optical Sensors); U.S. patent application Ser. No. 18/685,985 filed February 2024 titled Multi-Dimensional Signal Detection with Optical Sensors); each of which is incorporated herein by reference, disclose various methods for ultrasound beamforming, data processing, and image processing. The image and/or data representative of the environment at the medical device distal end 231 (or the fiber optic sensor device(s) 101) may then be displayed to the user on output device 208, which may include a computer display or the like.

As discussed above, the light receiving device 203 is in communication with the processing unit 209. The processing unit 209 receives information signals from the light receiving device 203 that are representative of the returned optical signal 212 received at the light receiving device 203. The processing unit 209 may also receive information signals from the light source control unit 207 that are representative of the initial optical signal 211 output by the light source 204. The processing unit 209 operates to process the information signals associated with the returned optical signal 212 (optionally in comparison with the information signals associated with the initial optical signal 211) to make determinations about an acoustic environment at the fiber optic sensor device 101, as discussed further below. Acoustic environment determinations may include the detection, identification, and interpretation of acoustic signals incident upon the fiber optic sensor device 101 or sensors 101 of the fiber optic sensor device array 201. Processing unit 209 may determine the presence and nature of acoustic signals incident upon the fiber optic sensor devices 101 of the fiber optic sensor device 101. Processing unit 209 may further serve to identify temperature and/or pressure signals contained in the returned optical signal 212, which may be further processed by the pressure/temperature determination unit 210.

Accordingly, the fiber optic sensor devices 101 may function to detect and/or measure environmental temperature and pressure as well as acoustic (e.g., ultrasound) signals, and provide optical signals that are representative of and consistent with the environmental temperature and pressure acoustic signals through an optical receive chain (e.g., optical devices 202C, 202B, 202D) to a light receiving device 203 configured to detect and/or receive the optical signals and provide electrical signals representative of and consistent with the optical signals to the processing unit 209 for processing and interpretation. Thus, the processing unit 209 may be configured to receive electrical signals that are representative of and consistent with the received/measured environmental temperature and pressure and acoustic signals and to process and interpret the electrical signals to reconstruct an image as well as determine environmental temperature and pressure.

The processing unit 209 may further be in communication with an acoustic control unit 222. The acoustic control unit 222 may be configured to provide control data to and receive signal data from the acoustic probe 245 and/or the acoustic transducers 221. The acoustic probe 245 may be configured for ex vivo or in vivo use and may include an AEG transducer or an array of AEG transducers (or any other suitable acoustic transducers) configured to generate and/or receive acoustic signals, such as ultrasound signals. The acoustic probe 245 may also include a mixed array of both AEG transducers (or any other suitable acoustic transducers) configured to generate and/or receive acoustic signals and optical sensors configured to receive optical sensors such as disclosed in U.S. Patent Publication Nos. US2022/0365036, US2023/0097639; US2022/0350022, and US2023/0148869, each of which is incorporated herein by reference. The one or more array elements of the first type (e.g., AEG transducers) may be used to form a first image. In parallel, the one or more array elements of the second type (e.g., the optical sensors) are used to detect acoustic echoes that can be used to form a second image. The second image that is generated by highly sensitive and broadband optical sensors may be used independently or can be combined with the first image to form an even further improved image. Because of the high sensitivity and broad bandwidth of optical sensors, the image produced by the optical sensors may have improved spatial resolution, improved penetration depth, improved signal-to-noise ratio (SNR), improved tissue harmonic imaging, and/or improved Doppler sensitivity.

The acoustic transducers 221 may be a component of a medical device system that is configured for in vivo deployment within the medium where the diagnostic or therapeutic procedure is or will be performed.

Typical ex vivo transducers 221 or probes 245 may be positioned on the patient's skin surface, such as commonly used for general imaging or for specific procedures, such as needle guidance, needle location, or needle placement.

The processing unit 209 is configured to use the information signals from the acoustic probe 245 or acoustic transducers 221 (as well as any other acoustic signal generator that may be connected to or in communication with the optical acoustic sensor system 200) as received by the fiber optic sensor device 101 to sense, track, and monitor the medical device distal end 231 as well as generate ultrasound images of the anatomy in the area of the procedure. In embodiments, the fiber optic sensor device 101 or sensor array 201 operates to receive/detect acoustic signals generated by the acoustic probe(s) 245 and/or the acoustic transducers 221, along with scattered signals and tissue harmonics. Imaging of the medium may be accomplished by processing unit 209 according to differences between acoustic signals output or transmitted by the acoustic probe(s) 245 and/or acoustic transducers 221 and corresponding acoustic signals received and/or detected by the acoustic(s) probes 245 and/or acoustic transducers 221 and the fiber optic sensor device 101. The signals detected may include the detected scattered signals and tissue harmonics. Portions of the medium through which the acoustic signals generated by the acoustic probe(s) 245 and/or acoustic transducers 221 travel may be imaged according to the detected acoustic signals.

The fiber optic sensor device 101 (or sensor array 201) receives the acoustic signal transmitted from the acoustic probe 245 and/or acoustic transducers 221. Based on the signals received from the fiber optic sensor device 101, the location of the fiber optic sensor device 101 (and thus, the location of the medical device distal end 231) may be calculated either by triangulation (e.g., based on the receipt of one or more acoustic signals transmitted from a known origin) and/or by coherent image formation. More details can be found in co-pending application U.S. patent application Ser. No. 18/382,984, titled TRANSPONDER TRACKING AND ULTRASOUND IMAGE ENHANCEMENT, filed on Oct. 23, 2023, and published as U.S. Patent Pub. No. 2024/0423482. The location of the fiber optic sensor device 101 may be overlayed on an ultrasound image of the anatomy to determine the relative location of the fiber optic sensor device 101 with respect to a known location of the acoustic probe 245 and/or acoustic transducers 221. Further, an ultrasound image of the surrounding anatomy may be coherently reconstructed according to a combination of acoustic signals received by the fiber optic sensor device 101 and by one or more of the acoustic probe 245 and/or the acoustic transducers 221. Such a combination may produce a better image quality than an image formed using acoustic probes 245 and/or acoustic transducers 221 alone.

In embodiments for tracking, sensing, and monitoring the medical device distal end 231, the optical acoustic sensor system 200 may include a plurality of acoustic probes 245 that are either fixed in place or have their locations tracked. Tracking, sensing, determining, and monitoring the location and movement of the medical device distal end 231 may be accomplished, for example, by identifying timing and/or directional differences between a plurality of acoustic signals detected by the fiber optic sensor device 101 and the acoustic transducer 221.

The temperature/pressure determination unit 210 may further be in communication with an ablation control system or ablation controller 235. The ablation control system 235 may further communicate/control the medical device distal end 231 and/or any other ablation tools that may be employed during an ablation procedure. The ablation control system 235 may use temperature and pressure information determined by the temperature/pressure determination unit 210 to assist in control and operation of an ablation procedure.

It will be understood that the configuration of the optical acoustic sensor system 200 as illustrated in FIG. 2 is provided by way of example. Different configurations may be employed without departing from the scope of this disclosure. For example, different arrangements of optical devices 202A/B/C/D, different numbers and arrangements of fiber optic sensor devices 101 and fiber optic sensor device arrays 201 may be employed. In embodiments, the light source control unit 207 and the acoustic control unit 222 may be incorporated or integrated within the processing system 250. Additional combinations of the components of the optical acoustic sensor system 200 may be selected as appropriate to achieve the functionality as described herein.

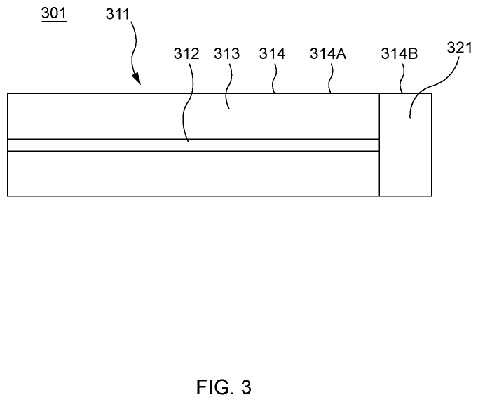

FIG. 3 illustrates a sensor fiber including a fiber optic sensor device and associated optical fiber. An apparatus, as illustrated in FIG. 3, may include a sensor fiber 301. The sensor fiber 301 may be an optical fiber configured with a fiber optic sensor device disposed on an end thereof. Sensor fiber 301 includes an optical waveguide 311 comprising a core 312 and a cladding structure 313. The optical waveguide 311 is configured to transmit or carry light therein, e.g., within the core 312. The core 312 is surrounded by and protected by the cladding structure 313. The optical waveguide 311 may be substantially cylindrical along its length and/or may be of another suitable shape. The core 312 may be substantially in the center of the cladding structure 313. In embodiments, the optical waveguide 311 may be an optical fiber and may include any materials common to optical fibers. For example, core 312 may include silica glass, polymer, or other appropriate material. The cladding structure 313 material may be selected to be responsive to, for example, changes in environmental temperature or pressure as well as ultrasound-induced pressure or strain. Changes in environmental temperature or pressure or the pressure or strain induced by ultrasound will introduce a deformation or refractive index changes, leading to variations in optical signals passing through the optical fiber. When used as a multimodal sensor, for example, the larger the variation in response to any of the parameters to be detected leads to higher sensitivity and a better detection limit. The cladding material may have at least one material property associated therewith, where the at least one material property may be a lower refractive index (RI) than a refractive index of the optical fiber core 312. Material properties such as the Young's modulus, photo-elastic coefficient, and thermal coefficient of the fiber core, cladding materials, and encapsulating structures, which may be identical or different materials, can be tailored for the application. A smaller Young's modulus and larger thermal and photo-elastic coefficients may be preferred for increased temperature sensitivity, pressure sensitivity, ultrasound sensitivity and for acoustic responsiveness. As used herein, sensitive or responsive to acoustic signals may refer to materials that have a relatively small Young's modulus (E), a relatively high photo-elastic coefficient, and/or a relatively large refractive index (n), for example, as compared to silica materials. Such features may also increase responsiveness or sensitivity to tissue pressure changes. As used herein, a relatively small Young's modulus may refer to a Young's modulus less than 3.0 GPa, less than 2.0 GPa, less than 1.2 GPa, or within a range between 1.2 and 0.8 GPa. A relatively high photo-elastic coefficient C (i.e., |C₁-C₂₁|) may refer to photo-elastic coefficients greater than C=2*10⁻¹² 1/Pa. A relatively large refractive index may refer to a refractive index greater than approximately 1.46 for optical signals that range between approximately 300 nm-2000 nm. Such materials may be selected to increase, improve, or optimize the ability of optical structures discussed herein to detect acoustic, pressure, and temperature signals.

Because the optical structures described herein are configured to detect temperature, pressure, and acoustic signals (e.g., ultrasound signals), the materials of which they are constructed may be selected to maximize or increase the sensitivity of the optical properties of the structures with respect to these parameters. For example, a material with a lower Young's module requires less stress to deform. In some applications, increased deformation may be undesirable. However, increased deformation in response to incident local tissue pressure and/or acoustic signals may amplify or increase detectable changes in optical signals that pass through the optical structures experiencing greater deformation. Similarly, increases in the photo-elastic effect are desirable in optical structures as described herein, but may be undesirable in different structures configured for different purposes. Finally, higher thermal coefficients may also be advantageous as they may produce a larger response to thermal changes in the tissue.

It should be understood that the optical fiber core 312 may be any suitable type of optical fiber core, such as those made from silica, silicon, optically transparent polymers, or the like. As a non-limiting example, if the optical fiber core 312 is made from silica (SiO₂), the cladding material may be MY-133, a low refractive index optical coating manufactured by MY Polymers Ltd. of Israel, or BIO-133, also a low refractive index optical coating manufactured by MY Polymers Ltd. of Israel. As a further non-limiting example, if the core is silicon, which has a higher RI than silica, the cladding structure 313 may be polyvinylidene fluoride (PVDF), polystyrene (PS), parylene, benzocyclobutene (BCB), MY-133, or BIO-133.

The optical waveguide 311 may be configured for single mode (SM) transmission or for multi-mode (MM) transmission, depending upon the form factor and laser and sensor wavelength tuning requirements, as a SM fiber will be smaller in size. For example, a single mode fiber configured to operate in a 1550 nm band may have a 50 μm cladding structure diameter and a core D=>4.2 μm. Such a fiber may be a polarization maintaining fiber. A Multimode fiber configured to operate in the 1550 nm band may have a core D=50 μm-60.5 μm and a 125 μm cladding diameter. In embodiments, a polymer fiber (e.g., PMMA, polystyrene) may be used. Such a fiber may have a larger diameter and a larger minimum bending radius than typical glass optical fibers. In other embodiments, a photonic crystal fiber (having a hollow structure/periodic pattern) may be used.

Disposed at an end of the sensor fiber 301 is an optical resonator structure 321. The optical resonator structure 321 is coupled to the end of the optical waveguide 311 and may include an optical resonator, such as a Fabry-Perot (FP) resonator, whispering-gallery mode resonator, micro-ring, micro-toroid, spiral resonator or a photonic crystal resonator integrated therein. The optical resonator structure 321 and other optical resonator structures described herein may include, in addition to the optical resonator, additional structures and components configured to facilitate the functionality of the optical resonator, as described below. The optical resonator is configured for receiving a first optical signal (e.g., light) supplied to it via the optical waveguide and providing a second optical signal back along the optical waveguide. The second optical signal may correspond to and represent environmental pressure, environmental temperature, and/or acoustic signals incident upon the optical resonator structure 321. As discussed above, environmental pressure, environmental temperature, and/or acoustic signals may cause physical deformation and/or material property alteration of the optical resonator structure 321. Accordingly, an optical signal provided along the optical waveguide 311 by the optical resonator structure may be altered by, influenced by, or otherwise indicative or representative of environmental pressure, environmental temperature, and/or acoustic signals and therefore may be used to characterize the environmental pressure, environmental temperature, and/or acoustic signals.

The sensor fiber 301 may further comprise an encapsulating structure 314, which may include, for example, an outer coating, shielding, protective outer layer, and/or fiber jacket. The encapsulating structure 314 is configured with a first portion 314A surrounding the optical waveguide and 311 and a second portion 314B that at least partially surrounds the optical resonator structure 321. The encapsulating structure 314 may include a polymer, such as parylene, MY-133, BIO-133, or other suitable polymer that is sensitive or responsive to environmental pressure, environmental temperature, and/or acoustic signals, as discussed above. The acoustic impedance of the encapsulating structure 314 may be selected to match an impedance of the optical resonator structure 321 so as to enhance the sensitivity of detection of acoustic signals. As used herein, “matching the impedance” may refer to selecting materials and/or structures that have acoustic impedances that match, generally it is well known to those of skill in medical ultrasound that acoustic impedances within 20% of one another provide an acceptable match. Closer matches in acoustic impedance lead to a better transmission of the acoustic signal (e.g., a smaller portion of the acoustic signal is reflected) and thus higher sensitivity. In embodiments, the first portion 314A surrounding the optical waveguide and 311 and a second portion 314B that at least partially surrounds the optical resonator structure 321 may comprise different materials selected for different purposes. For example, the first portion 314A may include an acoustically transmissive material, e.g., having an acoustic impedance selected to increase matching and thereby minimize reflection of acoustic signals. The second portion 314B may include acoustically, thermally, or pressure responsive/sensitive materials, as discussed above, to increase a response to environmental pressure, environmental temperature, and/or acoustic signals in the area of the optical resonator structure 321. Unless explicitly stated otherwise, all encapsulating structures discussed herein may include properties similar to those of encapsulating structure 314, including a first portion and a second portion comprising different materials selected for different purposes.

The optical resonator structure 321 is disposed at an end of the optical waveguide 311 and may therefore be referred to as a fiber-end sensor. The cladding structure 313 may have a first diameter and the optical resonator structure 321 may have a second diameter. The first diameter and the second diameter may or may not be substantially the same. Depending on the application, it may be advantageous to have the fiber substantially the same size or to have a significantly larger sensor than the fiber, such as a bulb like structure that may or may not be symmetrical. The increased size may further enhance the acoustic sensitive surface area of the sensor, increasing the overall sensitivity. As discussed above, the sensor fiber 301 may be compact as may be needed in view of the small form factor needed for certain medical applications, in some examples, wherein the first diameter and/or the second diameter are less than 200 microns, less than 175 microns, less than 150 microns, less than 130 microns, less than 100 microns, or less than 85 microns.

Aspects of the sensor fiber 301 and the optical resonator structure 321 may be selected and/or designed to address the extreme tissue temperatures (both hot and cold) that may occur during ablation. For example, cavity materials described herein, including polymer materials such as MY-133 or BIO-133 may operate successfully in temperatures ranging from −150° C. to 125° C. Optical fibers provided with dual acrylate coating may maintain performance at temperatures ranging from −60° C. to +85° C. Polyimide coatings may extend the temperature range of optical fiber to −60° C. to +330° C. Dielectric mirror coating meeting the environmental and durability requirements of MIL-C-48497 may be used, providing a working temperature range between-62° C. to 71° C. In embodiments, other suitable materials having different operating temperature ranges may also be selected.

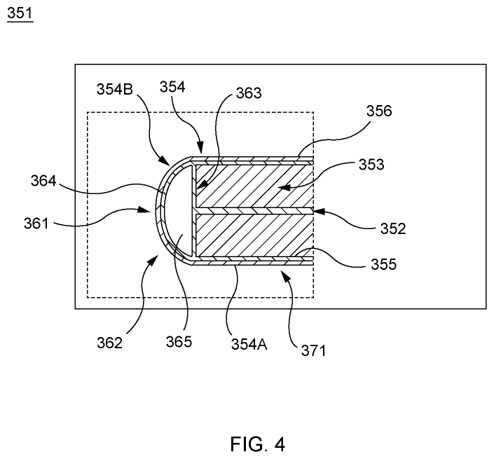

FIG. 4 illustrates a sensor fiber including an optical waveguide and optical resonator structure having a Fabry-Perot type resonator as an optical sensor. Sensor fiber 351 is an example of sensor fiber 301 and may include any of the features of sensor fiber 301 as described above. Sensor fiber 351 includes an optical waveguide 371 having a core 352 and a cladding 353. The core 352 may have a diameter in a range between 7 and 12 microns or a diameter of approximately 9 microns. These dimensions are provided as an example only and do not limit the sizes and diameters encompassed by embodiments of the present disclosure. The sensor fiber 351 may include an encapsulating structure 354, which may, for example, include an outer coating, protective outer layer, and/or fiber jacket, that encapsulates both the optical waveguide 371 and an optical resonator structure 361 disposed at an end of the optical waveguide 371. The encapsulating structure 354 may be a multi-layer structure, including, for example, an inner layer 355 and an outer layer 356. The inner layer 355 may include gold or any suitable reflective material layer for the optical wave while the outer layer 356 may include parylene, MY-133, BIO-133, or other suitable protective layer that may be acoustically transparent. The encapsulating structure 354 may include a first portion 354A that encapsulates or surrounds the optical waveguide 371 and a second portion 354B that encapsulates or surrounds the optical resonator structure 361. The encapsulating structure 354 may have features similar to those of encapsulating structure 314, including a first portion and a second portion of different materials. The optical resonator structure 361 may be configured with a Fabry-Perot resonator as an optical resonator 362. The optical resonator 362 includes a distal reflecting surface 364 and a proximal reflecting surface 363 arranged at either side of an optical cavity 365. The distal reflecting surface 364 and the proximal reflecting surface 363 may be constructed of any suitable reflective material. As shown in FIG. 4, the distal reflecting surface 364 and the proximal reflecting surface 363 are formed from and integral with the inner layer 355 of the encapsulating structure 354, and are thus formed of gold or other suitable reflective material. As illustrated in FIG. 4, the distal reflecting surface 364 may be curved and the proximal reflecting surface 363 may be substantially flat. This arrangement is by way of example only, and the distal and proximal reflecting surfaces 364/363 may be arranged with different shapes and/or configurations. In other embodiments, the distal reflecting surface 364 and the proximal reflecting surface 363 may be formed from different materials and/or may be structures separate from the encapsulating structure 354. The optical cavity 365 is disposed between the distal reflecting surface 364 and the proximal reflecting surface 363. The term “optical cavity,” as used herein, refers to a volume occupied by a material that provides minimal attenuation to light passing therethrough (e.g., having a high Q factor typically higher than 1000). The quality (Q) factor is a dimensionless parameter that describes the amount of damping within a resonator. A higher Q factor corresponds to a more sensitive resonator.

In optics, the Q factor of a resonant cavity is given by:

$$Q = \frac{2 π {f}_{0} E}{P} ,$$

where f₀ is the resonant frequency, E is the stored energy in the cavity, and

$$P = - \frac{dE}{dt}$$

is the power dissipated. The optical Q factor is equal to the ratio of the resonant frequency to the bandwidth of the cavity resonance. The average lifetime of a resonant photon in the cavity is proportional to the cavity's Q factor. Thus, a high Q factor represents low damping, with a high lifetime for a photon within the cavity.

The Q factor, as well as any other determinations of sensitivity and responsiveness, are ultimately limited by the choice of material used for the optical fiber core. A conventional Fabry-Pérot resonator may be formed uniformly from a single material, such as silica throughout the entire structure. Although silica, for example, has excellent optical transmission capabilities, it does not have equally exceptional acoustic sensitivity. Although numerous materials with superior acoustic sensitivity are known, such materials, on their own, may not make suitable replacements for silica and the like for optical fiber cores. The present invention adapts resonant actuators to take advantage of the acoustic sensitivity found in other materials.

The optical cavity 365 may be composed of a suitable material, such as a polymer. Polymer materials, such as MY-133 or BIO-133, with high acoustic transmissivity may be employed to enhance the sensitivity of the optical resonator structure, as discussed above. The optical resonator structure 361 may be configured to detect environmental pressure, environmental temperature, and/or acoustic signals. Acoustic signals incident upon the optical resonator structure, e.g., upon the distal reflecting surface 364, the proximal reflecting surface 363, and/or the optical cavity 365 may cause vibrations and/or other physical deformations of these structures, which may alter or influence their optical properties. Their optical properties may further be influenced by local temperatures and pressures. Further, due to the photo-elastic effect, the material properties of these structures may be altered and thus further change the optical properties. Accordingly, return optical signals provided to the optical waveguide 371 by the optical resonator structure 361 (e.g., in response to optical signals supplied via the optical waveguide 371) may be indicative of or representative of the acoustic signals incident upon the optical resonator structure 361 as well as the local temperature and pressure. More particularly, detected phase shifts of the light in the sensor beam, are indicative of environmental pressure, environmental temperature, and/or acoustic signals. With a polarization based sensor, a polarization analyzer will interpret the phase shift/delays between the different polarization components in order to generate the signal indicative of the sensed acoustic signals.

The temperature, pressure, and incident acoustic waves change either the geometry of the structure or the material properties that light “sees,” and those changes shift the optical signal that comes back through the fiber. Temperature changes cause the resonator and surrounding materials to expand or contract, which changes cavity dimensions and therefore the resonance. Temperature also changes refractive index directly. Those two effects again shift the resonant condition, so the returned optical signal varies with temperature.

For pressure and acoustic signals (including ultrasound), when local tissue pressure changes, or when an ultrasound wave impinges on the fiber end sensor, it applies a time varying pressure that slightly deforms the resonator region and the surrounding encapsulation. That deformation changes the resonator's optical path length, for example by changing the cavity length in a Fabry Perot resonator and by changing the spacing or curvature of the reflective surfaces. In parallel, stress in the materials produces a photoelastic effect that changes refractive index. Both effects change the resonance condition, so the “second optical signal” returned into the fiber is altered in a way that represents the incident temperature, pressure, or acoustic waveform.

In some embodiments, the optical resonator structure 361 is configured to sense temperature by translating thermally induced physical and optical-property changes into a measurable shift in the resonant response of the optical resonator 362. Temperature variations in the surrounding environment can cause dimensional changes in the materials that define the resonator region, including the encapsulating structure 354 and materials forming the proximal reflecting surface 363, distal reflecting surface 364, and the optical cavity 365. These dimensional changes can be driven by thermal expansion or contraction of the cavity material and adjacent layers, and may be particularly pronounced when the optical cavity 365 comprises a polymer selected for acoustic transmissivity. As the temperature changes, the effective optical path length of the resonator changes, thereby shifting the resonance condition of the Fabry Perot resonator 362.

In some examples, thermal expansion can change the separation between the proximal reflecting surface 363 and the distal reflecting surface 364, thereby changing the physical thickness of the optical cavity 365. Even very small changes in cavity thickness can shift the resonance because the resonant condition depends on both the cavity length and the refractive index within the cavity. In addition, temperature dependent expansion or contraction of the encapsulating structure 354, including the inner layer 355 and outer layer 356, can impose mechanical boundary conditions on the resonator region that further influence the effective cavity length or mirror geometry. For example, thermally induced strain in the second portion 354B can slightly alter the curvature or alignment of the reflecting surfaces 363 and 364, which can change coupling efficiency and the effective reflectivity profile experienced by light propagating in the optical waveguide 371.

Temperature can also directly modify the refractive index of one or more materials in the resonator region through the thermos-optic effect. In some embodiments, the refractive index of the optical cavity 365 changes with temperature, and the refractive index of portions of the optical waveguide 371, including the core 352 and cladding 353, may also vary with temperature. Because the resonance depends on the product of refractive index and cavity length, these thermos-optic changes can contribute to, or in some cases dominate, the overall resonance shift. Accordingly, a temperature change can produce a combined effect in which both the physical cavity thickness and the optical refractive index change concurrently, yielding a measurable and repeatable shift in the resonant response of the optical resonator 362, such as the Fabry Perot resonator.

In some embodiments, light conveyed by the optical waveguide 371 to the distal region of the sensor fiber 351 is partially reflected at the proximal reflecting surface 363 and the distal reflecting surface 364, producing an interference response that depends on the resonance of the optical resonator 362. As the temperature changes, the resulting shift in the resonance causes a corresponding change in one or more measured features of the returned optical signal, such as a change in intensity, wavelength, phase, or spectral position of a resonant peak or dip. In some examples, the optical sensor system 100B may therefore determine temperature based on this temperature dependent modulation of the returned optical signal. In other examples, the optical sensor system 100B may use calibration data to relate resonance shifts to absolute temperature or temperature change at the sensor location.

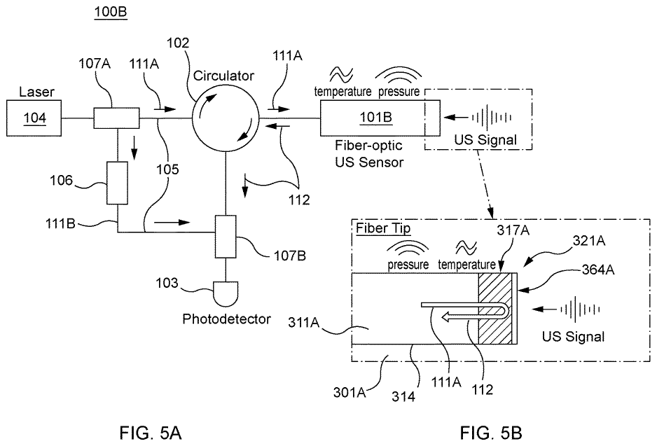

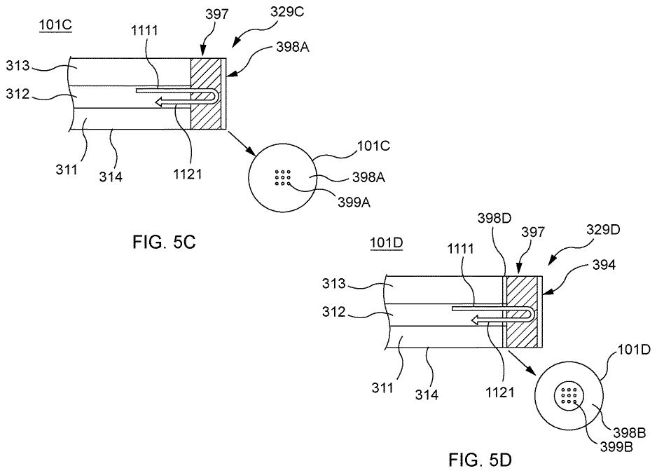

FIG. 5A illustrates an optical sensor system for use with a fiber optic sensor device according to embodiments herein. FIG. 5B illustrates an interferometer based optical sensor according to embodiments herein. The optical sensor system 100B of FIG. 5A is configured for use with an interferometer based fiber optic sensor device 101B, as shown in FIG. 5B.

The fiber optic sensor device 101B may include a fiber end sensor having an interferometer based acoustic sensor. The fiber optic sensor device 101B may include a sensor fiber 301A having an interferometer based fiber-end sensor structure 321A disposed at an end thereof, e.g., at the end of an optical waveguide 311A. Except where noted, the sensor fiber 301A may include features and structures consistent with those of sensor fiber 301. The interferometer based fiber-end sensor structure 321A may include, for example, a Mach-Zehnder (MZ) type of interferometer. In other examples, the interferometer based fiber-end sensor structure 321A may include other types of interferometers, such as, for example, a Michelson interferometer, a Fabry Perot interferometer, a Sagnac interferometer, a common path interferometers, a multimode interferometer, a polarization interferometers, or a Fizeau interferometer.

The interferometer based fiber-end sensor structure 321A is coupled to the end of the optical waveguide 311A. The interferometer based fiber-end sensor structure 321A may include, additional structures and components configured to facilitate the functionality of the interferometer based fiber-end sensor, as described below. The interferometer based fiber-end sensor is configured for receiving a first optical signal (e.g., light) supplied to it via the optical waveguide 311A and providing a second optical signal back along the optical waveguide 311A. The second optical signal may correspond to and represent environmental temperature, environmental pressure, and/or acoustic signals incident upon the interferometer based fiber-end sensor structure 321A. The environmental temperature, environmental pressure, and/or acoustic signals may cause physical deformation and/or material property alteration of the interferometer based fiber-end sensor structure 321A. Accordingly, an optical signal provided along the optical waveguide 311A by the interferometer based fiber-end sensor structure 321A may be altered by, influenced by, or otherwise indicative or representative of the environmental temperature, environmental pressure, and/or acoustic signals and therefore may be used to characterize the environmental temperature, environmental pressure, and/or acoustic signals.

The interferometer based fiber-end sensor structure 321A may include an acoustically, thermally, or pressure responsive polymer portion 317A including parylene or other suitable polymer. The acoustic impedance of the polymer portion 317A may be selected to match (e.g., within 1%, 5%, 10%, or 20%) the acoustic impedance of an encapsulating structure of the sensor fiber 301A to enhance the sensitivity of the fiber-end sensor structure 321A, as described above. A distal reflecting surface 364A is arranged at the distal end of the fiber-end sensor structure 321A and may be constructed of any suitable material, for example, gold. As shown in FIG. 5B the distal reflecting surface 364A is formed from gold and integral with polymer portion 317A.

The fiber-end sensor structure 321A is disposed at an end of the optical waveguide 311A and may therefore be referred to as a fiber end sensor. The optical waveguide 311A may have a first diameter and the fiber end sensor structure 321A may have a second diameter. The first diameter and the second diameter may be substantially the same and/or may have a ratio in a range between 1.05 and 0.95, a ratio in a range between 1.02 and 0.98, or a ratio in a range between 1.01 and 0.99. As discussed above, the sensor fiber 301A may be compact, e.g., wherein the first diameter and/or the second diameter are less than 200 microns, less than 175 microns, less than 150 microns, less than 130 microns, less than 100 microns, or less than 85 microns.

The optical sensor system 100B is configured for use with an interferometer based fiber optic sensor device 101B. The optical sensor system 100B may include a light source 104, such as a laser, a light reception device 103, such as a photodetector, one or more optical waveguides 105, an optical circulator 102, one or more frequency shifters 106, and one or more couplers 107. In operation, the light source 104 supplies the initial optical signal 111A to the fiber optic sensor device 101 via the optical waveguides 105, through a coupler/decoupler 107A, and through the optical circulator 102. The supplied initial optical signal 111A is returned by the fiber optic sensor device 101 back along the optical waveguide 105. The returned optical signal 112 travels via the optical waveguides 105 through the optical circulator 102 and a coupler/decoupler 107B and is received at the light reception device 103. The coupler/decoupler 107A serves to direct a portion of the initial optical signal 111A through the frequency shifter 106 as reference optical signal 111B to the coupler/decoupler 107B where it may be combined with the returned optical signal 112 for detection and comparison at the light reception device 103. As discussed above, environmental temperature, environmental pressure, and/or acoustic signals incident on the fiber optic sensor device 101 alter the optical characteristics (including the physical structure as well as the optical material properties) of the fiber optic sensor device 101. Such optical characteristic alterations may be measured according to changes in the returned optical signal 112 as compared to the reference optical signal 111B.

FIGS. 5C and 5D illustrate embodiments of fiber optic sensor devices that include fiber end facets configured to provide or enhance environmental parameter and acoustic measurement capabilities. Fiber optic sensor device 101C and fiber optic sensor device 101D each include at least an optical waveguide 311, a fiber core 312, a cladding structure 313, and an encapsulating structure 314. The fiber optic sensor device 101C includes an optical sensor structure 329C that includes an acoustically responsive polymer portion 397 and a facet substrate 398A located at a distal end thereof. The fiber optic sensor device 101D includes an optical sensor structure 329D that includes an acoustically responsive polymer portion 397, a facet substrate 398B disposed between the polymer portion 397 and the core 312 and cladding structure 313, and a distal end reflective surface 394 disposed at a distal end of the polymer portion 397.

In the fiber optic sensor device 101C, the facet substrate 398A is disposed at a distal end of the fiber optic sensor device 101C. The optical sensor structure 329C is formed by the polymer portion 397 and the facet substrate 398A. The facet substrate 398A includes one or more facet structures 399A, as shown in the cross-sectional view. The facet structures 399A may include microstructures responsive or sensitive to temperature, pressure, and/or acoustic signals. Such may include metasurfaces including patterns of small elements arranged to change the wavefront shape of the acoustic signals and maximize the detection of acoustic signals, acoustically responsive low-dimensional materials with optomechanical features selected to optimize acoustic response, e.g., features that are more prone to deformation when receiving acoustic signals, exhibit greater material responses to acoustic signals, and plasmonic structures patterned to amplify light-matter interactions, as described herein. Plasmonic structures may locally amplify incident light due to their plasmonic resonance. Each of these structural features may further perform differently when subject to temperature or pressure changes, thereby providing a means for environmental parameter measurement. The facet structures 399A operate as an optical sensor as described herein. During operation, the supplied optical signal 1111 reflects off of the facet substrate 398A and is returned to the system as the returned optical signal 1121. Because the facet structures 399A are acoustically, thermally, and/or pressure responsive, the returned optical signal 1121 is modified by changes in the facet structures 399A caused by incident acoustic signals and/or by changes in temperature or pressure. In embodiments, plasmonic resonance induced in a plasmonic meta-surface serving as the facet structures 399A or Mie resonance induced in a dielectric meta-surface serving as the facet structures 399A may be altered (e.g., shifted) by incident acoustic signals or by changes in temperature and pressure to provide detectable modifications in the returned optical signal 1121. The returned optical signal 1121 may then be interpreted by any of the systems described herein.

In the fiber optic sensor device 101D, the facet substrate 398B is disposed between the polymer portion 397 and core 312 and cladding structure 313. The optical sensor structure 329D is formed by the polymer portion 397, the facet substrate 398B, and the distal reflective surface 394. The facet substrate 398B includes one or more facet structures 399B, as shown in the cross-sectional view. The facet structures 399B may include acoustically, thermally, and/or pressure responsive microstructures similar to those described above with respect to facet structures 399A. The facet structures 399B operate to enhance, improve, or otherwise modify the acoustic, thermal, and/or pressure response of the optical sensor structure 329D. During operation, the supplied optical signal 1111 reflects off of distal reflective surface 394 and is returned to the system as the returned optical signal 1121. The polymer portion 397 and the distal reflective surface 394 are thermally, acoustically, and/or pressure responsive and the returned optical signal 1121 is modified according to acoustic signals incident upon these structures. Because the facet structures 399B are thermally, pressure, and/or acoustically responsive and both the supplied optical signal 1111 and the returned optical signal 1121 pass through the facet substrate 398B, the returned optical signal 1121 is further modified by changes in the facet structures 399B responsive to temperature, pressure, and/or incident acoustic signals. In embodiments, the facet structures 399B may be designed and/or selected to optimize coupling (e.g., decrease signal loss) and/or achieve critical coupling (e.g., eliminate signal loss) for the optical sensor structure 329D. Increased coupling in the optical sensor structure 329D serves to increase the amplitude of optical signals responsive to temperature, pressure, and/or incident acoustic signals. Thus, the returned optical signal 1121 may exhibit a higher signal to noise ratio. Further, temperature, pressure, and/or incident acoustic signals that cause deformation in the facet structures 399B may also server to alter the degree to which the facet structures 399B modify the coupling in the optical sensor structure 329D, thus providing another aspect of returned optical signal 1121 that is altered by incident acoustic signals for interpretation. The returned optical signal 1121 may then be interpreted by any of the systems described herein. Accordingly, the facet substrate 399B may serve to enhance, improve, or otherwise modify the thermal, pressure, and/or acoustic response of the optical sensor structure 329D.

In some embodiments, and with reference to FIGS. 5C, the distal sensing region may include additional engineered features configured to enhance coupling between incident acoustic signals and the optical resonator structure 361. By way of example, the facet structures 399A (and similarly, facet structures 399B) may include metasurfaces, such as patterns of small elements arranged on or within a facet substrate (e.g., facet substrate 398B) to modify the phase or spatial distribution of incident acoustic waves. In this manner, the metasurface type facet structures can direct a greater portion of acoustic energy toward the optical cavity 365 and associated reflecting surfaces 363, 364, thereby increasing acoustic induced modulation of the resonator response.

In some embodiments, the facet structures 399A, 399B may additionally or alternatively include one or more acoustically responsive materials, for example as a thin layer, membrane, or low dimensional material associated with the distal region (e.g., coupled to the second portion 354B of the encapsulating structure 354 and or integrated with a facet substrate). Such materials may be selected to exhibit increased compliance or enhanced photoelastic response, such that incident acoustic signals produce an increased mechanical deformation and or refractive index perturbation within the resonator region. By way of example, the acoustically responsive materials may include compliant polymers or elastomers, thin film or membrane layers, piezoelectric materials, and or low dimensional nanomaterials, each of which can be selected to increase displacement or stress induced optical changes in response to acoustic pressure waves. This may increase modulation of the optical path length of the optical resonator 362 without requiring substantial changes to the overall sensor geometry.

In some embodiments, the facet structures 399A, 399B may include plasmonic structures disposed at or near the distal end of the sensor fiber 351. Plasmonic structures are nanoscale metallic features that interact very strongly with light by coupling it to oscillations of free electrons in the metal, called surface plasmons. When light hits certain metals such as gold or silver at specific wavelengths, the conduction electrons at the surface collectively oscillate. Based on the engineered geometry, this produces a resonance known as localized surface plasmon resonance. At resonance, the electromagnetic field becomes highly concentrated near the surface of the structure, often much stronger than the incident light. These plasmonic structures are typically formed as very small patterns such as nanoparticles, nanoholes, nanorods, gratings, or thin films with periodic features. Their size, shape, and spacing determine the resonant behavior, meaning they can be designed to respond to specific wavelengths or environmental changes. In sensing applications, a very small physical change such as slight deformation, a tiny refractive index shift, or temperature variation may cause a disproportionately large change in the optical signal. Thus, the plasmonic structures may be used to improve sensitivity in detecting acoustic signals, pressure, temperature, or biochemical changes.

In some embodiments, the temperature dependent behavior of the facet structures 399A, 399B can be configured to increase the sensitivity and resolution of temperature measurements by amplifying the underlying thermos-optic and thermal expansion effects. For example, materials included within the facet structures may be selected to have relatively high coefficients of thermal expansion and/or large thermos-optic coefficients, such that small temperature changes produce larger variations in cavity length and refractive index within the optical cavity 365. In addition, geometries associated with the facet structures, including metasurface type features, acoustically responsive materials, and/or plasmonic structures, may be configured to mechanically amplify thermally induced strain, for example by concentrating expansion or deformation in the region proximate the reflecting surfaces 363 and 364. This can result in a greater effective change in optical path length for a given temperature variation. Accordingly, the combined use of material selection and structural design within the facet structures 399A, 399B can improve signal to noise ratio and enable detection of smaller temperature variations.

The facet structures 399A and 399B are illustrated in FIGS. 5C and 5D as being incorporated into fiber optic sensor devices 101C and 101D. Such facet substrates are not limited to use with optical sensors having the interferometer based structure and operation of fiber optic sensor devices 101C and 101D and may be incorporated into any of the fiber optic sensor devices discussed herein.

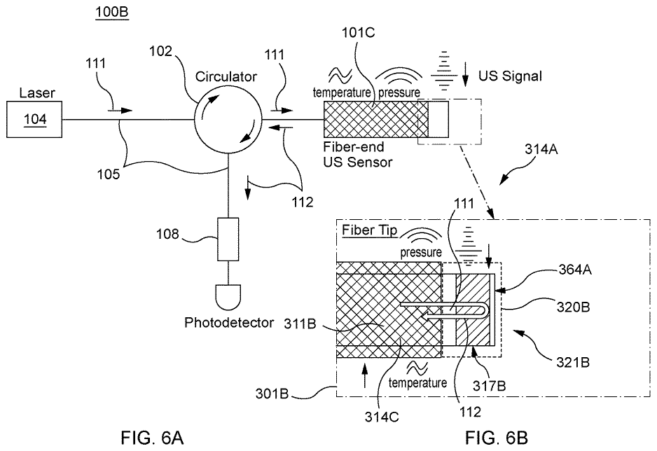

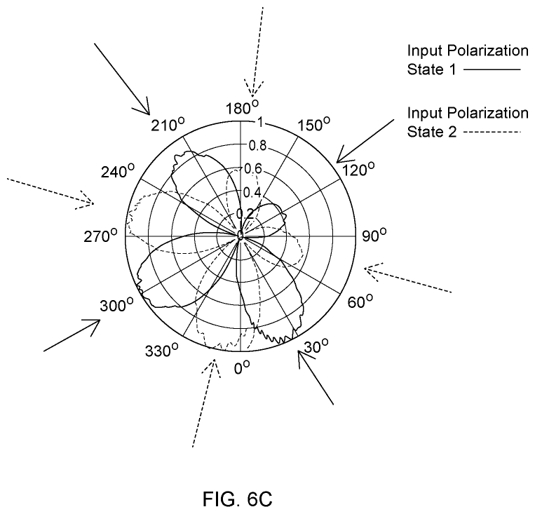

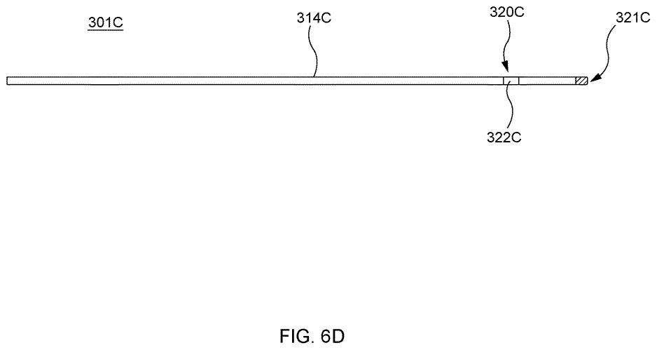

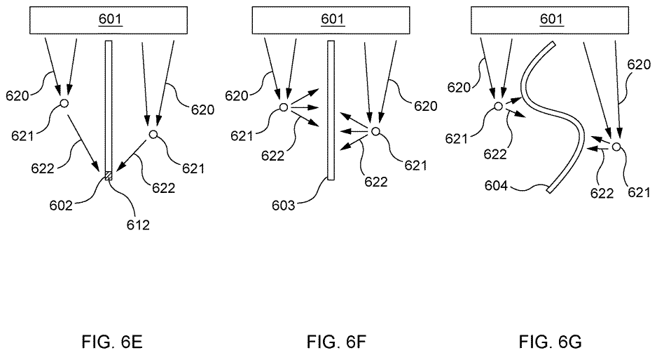

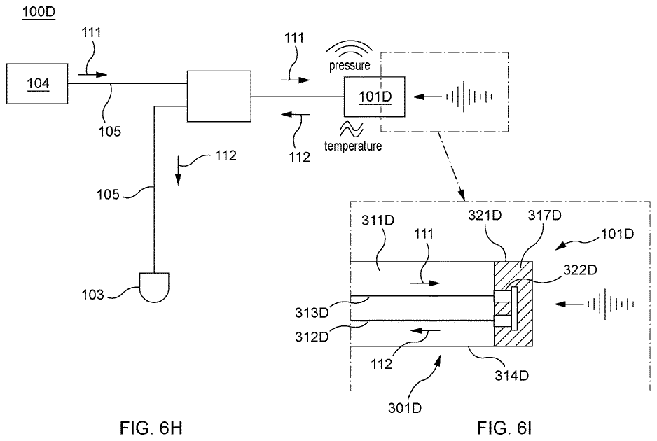

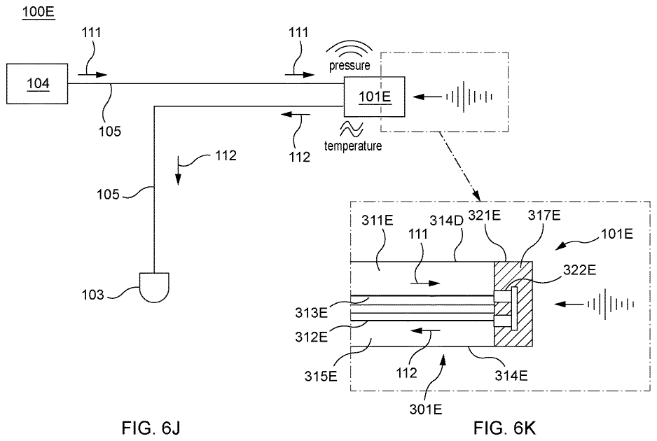

FIG. 6A illustrates an optical sensor system for use with a fiber optic sensor device according to embodiments herein. FIG. 6B illustrates a polarization based optical sensor according to embodiments herein. The optical sensor system 100B is configured for use with a polarization based fiber optic sensor device 101C. The fiber optic sensor device 101C may include a fiber end sensor having a polarization based acoustic sensor. The fiber optic sensor device 101C may include a sensor fiber 301B having a polarization based fiber-end sensor structure 321B disposed at an end thereof, e.g., at the end of an optical waveguide 311B. In further embodiments, as discussed below, the polarization based fiber-end sensor structure 321B may be disposed at any location along the sensor fiber 301B. Except where noted, the sensor fiber 301B may include features and structures consistent with those of sensor fiber 301. The sensor fiber 301B includes an encapsulating structure 314C, which may include, for example, an outer coating, protective outer layer, and/or fiber jacket. The encapsulating structure 314C may include a material selected to have a relatively high acoustic impedance mismatch with the cladding structure of the sensor fiber 301B. Accordingly, where the sensor fiber 301B is covered by the encapsulating structure 314C, incident acoustic signals may be reflected. The polarization based fiber-end sensor structure 321B may be exposed by a window 320B defined by a lack of encapsulating structure 314C and may include a polymer portion 317B comprising an acoustically responsive polymer and a distal reflective surface 364B configured to reflect the initial optical signal 111 as a reflected optical signal 112. In embodiments, the encapsulating structure 314C may also provide a thermal and/or pressure barrier between the local environment and the fiber end sensor structure. The polarization based fiber-end sensor structure 321B is configured for receiving a first optical signal (e.g., light) supplied to it via the optical waveguide 311B and providing a second optical signal back along the optical waveguide 311B. The second optical signal may correspond to and represent an acoustic signal incident upon the polarization based fiber-end sensor structure 321B. The incident acoustic signal may cause physical deformation and/or material property alteration of the polarization based fiber-end sensor structure 321B. Further, temperature and pressure variations in an environment local to the exposed window may also alter the properties of the polarization based fiber-end sensor structures in a manner that alters or influences the optical signal carried therein. Accordingly, an optical signal provided along the optical waveguide 311B by the polarization based fiber-end sensor structure 321B may be altered by, influenced by, or otherwise indicative or representative of the local temperature or pressure or an incident acoustic signal and therefore may be used to characterize the incident acoustic signal. In the polarization based fiber-end sensor structure 321B, the incident acoustic signal may cause stress in the polymer portion 317B that results in one or more of birefringence and a rotation of the polarization of the light passing through the polymer portion 317B changes in the polarization of the light carried by the optical waveguide 311B, which may be detected and analyzed by the optical sensor system 100B as discussed below. This phenomenon may be altered or influenced by local temperature changes and/or by a baseline pressure in the local tissue. Further, temperature changes and/or pressure changes in the local tissue may affect the polarization of light carried by the optical waveguide 311B in the absence of acoustic signals.

The optical sensor system 100B includes a light source 104, such as a laser, a light reception device 103, such as a photodetector, one or more optical waveguides 105, an optical circulator 102, and a fiber optic sensor device 101C. In operation, the light source 104 supplies the initial optical signal 111 to the fiber optic sensor device 101C via the optical waveguides 105 and through the optical circulator 102. The supplied initial optical signal 111 is returned by the fiber optic sensor device 101C back along the optical waveguide 105. The returned optical signal 112 travels via the optical waveguides 105 through the optical circulator 102, through the polarization analyzer 108, and is received at the light reception device 103. Use of the polarization analyzer 108 permits the determination of the polarization difference between the initial optical signal 111 and the returned optical signal 112. As discussed above, temperature, pressure, and/or acoustic signals incident on the fiber optic sensor device 101C alter the optical characteristics (including the physical structure as well as the optical material properties) of the fiber optic sensor device 101B and cause an alteration in the polarization of the returned optical signal 112. Such polarization changes may be measured according to differences in the returned optical signal 112 and the initial optical signal 111 as determined according to the photodetector.

In embodiments, the angular sensitivity of the polarization based fiber-end sensor structure 321B may be subject to differences in the polarization of the initial optical signal 111. Depending on the polarization of the initial optical signal 111, the angle of incident acoustic signals to which the polarization based fiber-end sensor structure 321B is most sensitive may be altered, as shown in FIG. 6C. Accordingly, in embodiments, a control system associated with the optical sensor system 100B may be configured to adjust or optimize the polarization of the initial optical signal 111, such as from input polarization state 1 to input polarization state 2 to increase thermal, pressure, and/or acoustic sensitivity, maximize signal to noise ratio, or reduce drift and polarization fading.

In an example related to acoustic sensitivity, FIG. 6C illustrates the direction in which input polarization state 1 and input polarization state 2 are most sensitive to incoming acoustic signals. The solid arrows correspond to the directions in which input polarization state 1 is most sensitive, and the dashed arrows correspond to input polarization state 2. Thus, the lobes of the polarization states provide the highest acoustic sensitivity. Because the most sensitive directions change with polarization, the system can choose a polarization state that aligns with the direction the acoustic wave is expected to come from. Accordingly, an input polarization state may be selected and implemented to align with an expected direction of acoustic signals or with a direction in which acoustic sensitivity (or, in other examples, temperature or pressure sensitivity) is most desired.

This may permit the optical sensor system 100B to optimize performance of the fiber-end sensor structure 321B according to an incoming direction of an acoustic signal. The angular sensitivity of the polarization based fiber-end sensor structure 321B is not reliant on the structure of the fiber-end sensor structure 321B. In embodiments, a polarization maintaining fiber may be used. After the polarization state is selected and implemented, it is maintained by the optical signal. In embodiments, an adjustable fiber component may be used to provide an adjustable polarization state. In an embodiment, the polarization state may be adjusted during use to account for changing conditions (such as movement of an acoustic transducer 221 generating the acoustic signal and/or movement, rotation, etc., of the fiber-end sensor structure 321B.) Further benefits of the polarization based fiber-end sensor structure 321B may include a simplified sensor structure and no wavelength locking requirements. Moreover, the angular sensitivity is not dependent on changing the physical structure of the fiber end sensor itself. Instead, the polarization maintaining fiber may be used to preserve the chosen polarization state as the light propagates, or an adjustable polarization component (like a polarization controller) may be used to set and adjust the polarization. If the geometry changes during use, for example the acoustic transducer moves or the fiber rotates, the system can readjust the polarization to keep the sensitivity lobe aligned with the acoustic source.

FIG. 6D illustrates a further embodiment of a fiber-based optical sensor consistent with embodiments hereof. A sensor fiber 301C may be an optical fiber configured similarly to sensor fiber 301, including an optical waveguide comprising a core and a cladding structure, as described herein. The sensor fiber 301C may have a fiber end sensor structure 321C disposed on an end thereof. The fiber end sensor structure 321C may include any of the fiber end sensor structures discussed herein, including optical resonator structures, interferometer structures, acoustically responsive fiber end facet structure and polarization based structures. The sensor fiber 301C may further include an encapsulating structure 314C configured to reflect incident acoustic signals and a window 320C representing a gap or exposure area that lacks encapsulating structure 314C. The window 320C may expose a polarization based optical sensor structure 322C, e.g., as discussed with respect to FIG. 6B. In embodiments, the polarization based optical sensor structure 322C is formed from the cladding structure and the core of the fiber of which it is a part. That is, the polarization based optical sensor structure 322C may be defined by exposure to the local tissue environment (e.g., pressure and temperature) and incident acoustic signals created by the lack of acoustic shielding at the window 320C, rather than any additional structure within the fiber. The sensor fiber 301C may include any number of windows 320C and polarization based optical sensor structures 322C disposed along its length. Thus, the sensor fiber 301C may include a plurality of optical based acoustic sensor structures, including both fiber end sensor structures 321C and polarization based optical sensor structures 322C configured for mid-fiber location. In further embodiments, the window 320C may be sized along the length of the fiber sufficiently that it will operate as a line sensor as opposed to a point sensor as will be discussed in greater detail below. The line sensor may be a straight line sensor or a curved line receiver. In further embodiments, the sensor fiber 301C may be configured with one or more polarization based optical sensor structures 322C disposed along its length while not including any fiber end sensor structures 321C.

Each of the fiber end sensor structure 321C and the polarization based optical sensor structures 322C may be used to facilitate environmental parameter measurement as well as imaging, location, guidance, and tracking, as described herein. In embodiments, a polarization based optical sensor 322C may be configured, e.g., by size/shape, to facilitate this functionality. For example, a longer polarization based optical sensor structure 322C may increase image quality, acting as a line sensor and the line may be straight or curved and/or may capture temperature/pressure over a larger area. In another example, multiple polarization based optical sensor structures 322C may be used to facilitate tracking methods (multiple sensors along a device may assist with orientation determination, for example.) In another example, multiple polarization based optical sensor structures 322C may be used to facilitate pressure or temperature mapping, e.g., by recording temperatures and/or pressures at multiple locations along the length of the sensor fiber 301C. Multiple sensors (e.g., polarization sensors) located along the length of a sensor fiber may be referred to herein as “distributed sensors.” When distributed sensors are used, the distances between them may be fixed to ensure that the locations of temperatures, pressures, and acoustic signals measured thereby can be understood.

In some embodiments, the polarization window portion may also work as a fiber optic sensor device that detects scattered acoustic signals and/or tissue harmonics. When the fiber optic sensor device is positioned within an imaging area of interest, it may receive weak harmonic or scattered acoustic signals that are unable to propagate very far. The fiber optic waveguide may convey optical signals corresponding to the received acoustic signals to a system processor (e.g., processing unit 209). The system processor may use the received optical signals to reconstruct the ultrasound image of the anatomy surrounding the sensor with a delay and sum beamforming method or other suitable image reconstruction method, as discussed in more detail in corresponding titled TRANSPONDER TRACKING AND ULTRASOUND IMAGE ENHANCEMENT and filed on Oct. 23, 2023. With this data, the system processor may generate an image of better quality than one generated solely based on the pulses emitted and received by an acoustic probe. In embodiments, the system processor may construct an image based solely on the optical signals received from one or more fiber optic sensor devices. In embodiments, the optical signals received from one or more fiber optic sensor devices may be used in conjunction with the acoustic signals received by a traditional ultrasound probe.

This principle is illustrated in greater detail in FIGS. 6E-6G. As shown in FIG. 6E, an acoustic probe 601 may be used to transmit acoustic signals 620 into an area of interest. The acoustic probe 601 may function as a traditional acoustic probe to detect reflections of the acoustic signals 620 for imaging purposes. These images may be enhanced by additional information obtained by one or more fiber optic sensor devices. The fiber optic sensor device 612 of the sensor fiber 602 may correspond to any of the fiber end optical sensor structures discussed herein and may receive acoustic signals 622. The acoustic signals 622 may result from reflection, scattering, and/or tissue harmonics. As shown in FIG. 6E, the acoustic signals 622 are generated from points 621 within the area of interest. The fiber optic sensor device 612 may be configured to receive acoustic signals 622 from any direction, as discussed herein. As shown in FIG. 6F, the sensor fiber 603 may be configured to act as a polarization based optical sensor, as discussed herein, and may receive acoustic signals 622 from directions lateral to the axis of the sensor fiber 603. As used herein, “lateral” refers to all directions that are not parallel to the axis of the sensor fiber 603. As shown in FIG. 6F, the acoustic signals 622 may be received by the sensor fiber 602 at any exposed portion along its length and from any direction, as discussed with respect to FIG. 6D. Further, as discussed with respect to and shown in FIG. 6C, the polarization of the sensor fiber 602 may be selected or adjusted to accommodate an expected or desired radial angle of incidence of the acoustic signals 622. FIG. 6G further illustrates the sensor fiber 604, which may curve within the area of imaging interest. Similar to the sensor fiber 603, the sensor fiber 604 may detect incident acoustic signals 622 lateral, substantially lateral, or from any direction relative to the axis of the sensor fiber 604.

Detection of lateral signals at multiple points along the length of the sensor fiber 604 may enhance an ability to track and/or locate the sensor fiber 604 when it is disposed within a medium (e.g., within a human body during a medical procedure). For example, as shown in FIG. 6G, multiple signals incident along the length of the sensor fiber 604 may enhance an ability to determine the location of different portions of the sensor fiber 604 along its length and therefore to identify the location of the entire sensor fiber 604, and not just a tip region. For example, as shown in FIG. 6G, multiple signals incident along the length of the sensor fiber 604 may enhance an ability to determine the location of different portions of the sensor fiber 604 and therefore to identify curvature of the sensor fiber 604 with greater accuracy. In embodiments, pressure and temperature measurement obtained at various locations along the length of the sensor fiber 604 may be combined with location measurements for temperature and/or pressure mapping purposes.

FIGS. 6H and 6I illustrate an optical sensor system for use with a fiber optic sensor device according to embodiments herein. FIG. 6I illustrates an optical resonator based optical sensor configured for use with a multi-core optical fiber according to embodiments herein. The optical sensor system 100D of FIG. 6H is configured for use with the multi-core optical resonator based fiber optic sensor device 101D, as shown in FIG. 6I. In further embodiments, other optical sensors discussed herein, including, for example, interferometer based sensors, may be employed in a multi-core optical fiber based system.

The fiber optic sensor device 101D may include a fiber end sensor having an optical resonator based sensor as described herein. The fiber optic sensor device 101D may include a sensor fiber 301D having an optical resonator based fiber-end sensor structure 321D disposed at an end thereof, e.g., at the end of an optical waveguide 311D. Except where noted, the sensor fiber 301D may include features and structures consistent with those of sensor fibers 301 and 351. The optical resonator based fiber-end sensor structure 321D is coupled to the end of the optical waveguide 311D. The optical resonator based fiber-end sensor structure 321D may include an optical resonator sensor 322D, in addition to additional structures and components configured to facilitate the functionality of the optical resonator sensor 322D, as described below. The optical resonator based fiber-end sensor 322D, schematically illustrated in FIG. 6I, may be waveguide-coupled such that it is configured for receiving an initial optical signal 111 (e.g., light) supplied to it via a first optical core 313D of the sensor fiber 301D and providing a returned optical signal 112 back along a second optical core 312D of the sensor fiber 301D. The second optical signal may correspond to and represent a temperature, pressure, and/or acoustic signal incident upon the optical resonator based fiber-end sensor structure 321D. The temperature, pressure, and/or incident acoustic signal may cause physical deformation and/or material property alteration of the optical resonator based fiber-end sensor structure 321D. Accordingly, an optical signal provided along the second optical core 312D by the optical resonator based fiber-end sensor structure 321D may be altered by, influenced by, or otherwise indicative or representative of the temperature, pressure, and/or acoustic signal and therefore may be used to characterize the temperature, pressure, and/or acoustic signal.

The optical resonator based fiber-end sensor structure 321D may include an acoustically, thermally, or pressure responsive polymer portion 317D including parylene or other suitable polymer that is sensitive to acoustic, thermal, and/or pressure signals. The acoustic impedance of the polymer portion 317D may be selected to match (e.g., within 1%, 5%, 10%, or 20%) of the acoustic impedance of an encapsulating structure or cladding structure 314D of the sensor fiber 301D to enhance the sensitivity of the optical resonator based fiber-end sensor structure 321D, as described above.

The fiber-end sensor structure 321D is disposed at an end of the optical waveguide 311D and may therefore be referred to as a fiber end sensor. The encapsulating or cladding structure 314D may have a first diameter and the fiber end sensor structure 321D may have a second diameter. The first diameter and the second diameter may be substantially the same and/or may have a ratio in a range between 1.05 and 0.95, a ratio in a range between 1.02 and 0.98, or a ratio in a range between 1.01 and 0.99. As discussed above, the sensor fiber 301D may be compact, e.g., wherein the first diameter and/or the second diameter are less than 200 microns, less than 175 microns, less than 150 microns, less than 130 microns, less than 100 microns, or less than 85 microns. With very small fiber diameters, increasing the diameter of the fiber sensor end may further enhance acoustic sensitivity.

The optical sensor system 100D is configured for use with the resonator based fiber optic sensor device 101D. The optical sensor system 100D may include a light source 104, such as a laser, a light reception device 103, such as a photodetector, one or more optical waveguides 105, and a multi-core fiber fan-out coupler 109. In operation, the light source 104 supplies the initial light signal 111 to the fiber optic sensor device 101D via the optical waveguide 105, through the multi-core fiber fan-out coupler 109. The supplied initial optical signal 111 travels to the optical resonator based fiber-end sensor structure 321D via a first optical core 313D, where it may be affected by an incident acoustic signal, and then is returned by the second optical core 312D as a returned optical signal 112. The returned optical signal 112 travels via the optical waveguides 105 through the fan-out coupler 109 to be received at the light reception device 103. As discussed above, temperature, pressure, and/or acoustic signal signals incident on the fiber optic sensor device 101D alter the optical characteristics (including the physical structure as well as the optical material properties) of the fiber optic sensor device 101D. Such optical characteristic alterations may be measured from the returned optical signal 112 to measure properties and characteristics of the temperature, pressure, and/or acoustic signals. In the embodiment of FIG. 6H, it is not necessary to provide the initial optical signal 111 to the light reception device 103 to measure the optical characteristic alterations, for example, because the parameters of the initial optical signal 111 are known by the system.

The multi-core fiber fan-out coupler 109 serves to couple the single core optical waveguides 105 to the multi-core optical waveguide 311D. Thus, the initial optical signal 111 and the returned optical signal 112 may travel in separate optical cores in the multi-core optical waveguide 311D. As compared to the optical sensor system 100B, use of the multi-core fiber fan-out coupler 109 and multi-core optical waveguide 311D in the optical sensor system 100D may eliminate the need for an optical circulator. Such a design may be advantageous for several reasons. For example, the multi-core fiber fan-out coupler 109 of the optical sensor system 100D may be smaller, lighter, and/or less expensive than an optical circulator, which may permit more flexibility when incorporating the fiber optic sensor device 101D into a device or apparatus. In embodiments, other suitable optical couplers configured for coupling single core optical fibers to multi-core optical fibers may take the place of the multi-core fiber fan-out coupler 109.

FIGS. 6J and 6K illustrate an optical sensor system for use with a fiber optic sensor device according to embodiments herein. FIG. 6K illustrates an optical resonator based optical sensor configured for use with a pair of single core optical fibers according to embodiments herein. The optical sensor system 100E of FIG. 6J is configured for use with the dual fiber optical resonator based sensor 101E, as shown in FIG. 6K. In further embodiments, other optical sensors discussed herein, including, for example, interferometer based sensors may be employed in a dual optical fiber based system.

The fiber optic sensor device 101E may include a fiber end sensor having an optical resonator based sensor as described herein. The fiber optic sensor device 101E may include a sensor fiber 301E having an optical resonator based fiber-end sensor structure 321D disposed at an end thereof. Except where noted, the sensor fiber 301E may include features and structures consistent with those of sensor fibers 301 and 351.

The fiber optic sensor device 101E may include a dual optical fiber structure. The fiber optic sensor device 101E may include a first optical waveguide 311E having a first fiber optical core 313E and a second optical waveguide 315E having a second fiber optical core 312E. Each of the first optical waveguide 311E and the second optical waveguide 315E may be individual optical fibers and may each have a separate cladding structure 314E. The first optical waveguide 311E and the second optical waveguide 315E may be coupled together. For example, the first optical waveguide 311E and the second optical waveguide 315E may be coupled via glue or other adhesive.

The optical resonator based fiber-end sensor structure 321E is coupled to the end of both the first optical waveguide 311E and the second optical waveguide 315E. The optical resonator based fiber-end sensor structure 321E may include an optical resonator sensor 322E, in addition to additional structures and components configured to facilitate the functionality of the optical resonator sensor 322E, as described below. The optical resonator based fiber-end sensor 322E, schematically illustrated in FIG. 6K, may be waveguide-coupled such that it is configured for receiving an initial optical signal 111 (e.g., light) supplied to it via a first optical core 313E of the first optical waveguide 311E and providing a returned optical signal 112 back along a second optical core 312E of the second optical waveguide 315E. The returned optical signal 112 may correspond to and represent a temperature, pressure, and/or acoustic signal incident upon the optical resonator based fiber-end sensor structure 321E. The temperature, pressure, and/or acoustic signal may cause physical deformation and/or material property alteration of the optical resonator based fiber-end sensor structure 321E. Accordingly, an optical signal provided along the second optical core 312E by the optical resonator based fiber-end sensor structure 321E may be altered by, influenced by, or otherwise indicative or representative of the acoustic signal and therefore may be used to characterize the incident acoustic signal. In the embodiment of FIG. 6J, it is not necessary to provide the initial optical signal 111 to the light reception device 103 to measure the optical characteristic alterations, for example, because the parameters of the initial optical signal 111 are known by the system.

The optical resonator based fiber-end sensor structure 321E may include an acoustically, thermally, or pressure responsive polymer portion 317E including parylene or other suitable polymer that is sensitive to temperature, pressure, and/or acoustic signals. The acoustic impedance of the polymer portion 317E may be selected to match (e.g., within 1%, 5%, 10%, or 20%) of the acoustic impedance of an encapsulating structure (or cladding structure) of the sensor fiber 301E to enhance the sensitivity of the optical resonator based fiber-end sensor structure 321E, as described above.

The optical sensor system 100E is configured for use with the resonator based fiber optic sensor device 101E. The optical sensor system 100D may include a light source 104, such as a laser, a light reception device 103, such as a photodetector, one or more optical waveguides 105. The one or more optical waveguides 105 may be structurally bound to one another to form the first optical waveguide 311E and the second optical waveguide 315E of the sensor fiber 301E and may be separated to couple with the light source 104 and the light reception device 103. In embodiments, a coupler or other device may be used to facilitate the junction. In operation, the light source 104 supplies the initial light signal 111 to the fiber optic sensor device 101E via the optical waveguide 105. The supplied initial optical signal 111 travels to the optical resonator based fiber-end sensor structure 321E via the first optical waveguide 311E, where it may be affected by an incident acoustic signal, and then is returned by the second optical waveguide 315E as a returned optical signal 112. The returned optical signal 112 travels via the optical waveguides 105 to be received at light reception device 103. As discussed above, temperature, pressure, and/or acoustic signal incident on the fiber optic sensor device 101E alter the optical characteristics (including the physical structure as well as the optical material properties) of the fiber optic sensor device 101E. Such optical characteristic alterations may be measured from the returned optical signal 112.

The dual fiber design of the sensor fiber 301E eliminates the need for a circulator or a multi-core fan-out coupler. Such a design may be advantageous for several reasons. For example, eliminating a multi-core fiber fan-out coupler and an optical circulator may provide a smaller, lighter, and/or less expensive system, which may permit more flexibility when incorporating the fiber optic sensor device 101E into a device or apparatus.

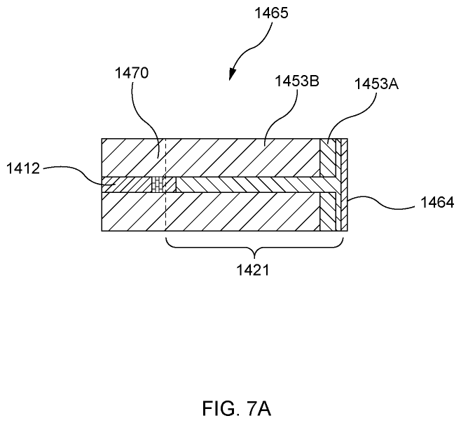

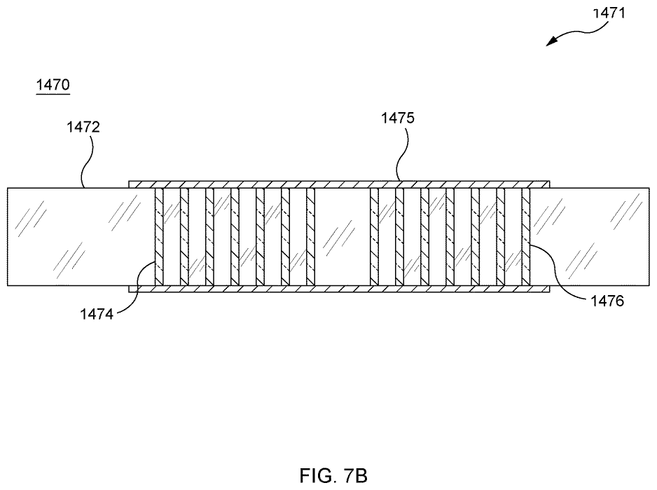

FIG. 7A illustrates an optical resonator structure including an in-fiber Bragg grating consistent with embodiments hereof. The optical resonator structure 1421 may be provided in combination with any of the sensor fibers discussed herein. The optical resonator structure 1421 includes a distal reflecting surface 1464, an elongated optical cavity 1465 comprising a distal cladding structure 1453A, and a proximal cladding structure 1453B, and a Bragg grating 1470. The Bragg grating 1470 is integrated within the structure of the core 1412 and defines variations in the refractive index of the core 1412, thereby producing a structure that may reflect light of specific wavelengths, e.g., the Bragg wavelength. The optical resonator structure 1421 may operate as a hybrid Fabry-Perot resonator or any other fiber-end sensor described herein.

FIG. 7B illustrates the fiber Bragg Grating 1470 in greater detail. In fiber Bragg gratings (FBGs) the Bragg wavelength is a known constant under stable conditions, but can shift due to variations in temperature and strain. For constant temperatures, the measurement of Bragg wavelength shift yields a highly accurate measurement of applied strain. Conversely, for constant strain, the measurement of Bragg wavelength can yield a highly accurate measurement of temperature. FBGs can be highly effective for measuring small vibrations and small changes in temperature. FBGs, therefore, may be used to measure environmental parameters such as temperature and pressure, as well as acoustic signals

As shown in FIG. 7B, the optical fiber with a fiber Bragg grating 1471 includes an optical fiber core 1472 with a pair of fiber Bragg gratings 1474, 1476 formed therein, such that each of the fiber Bragg gratings 1474, 1476 is spaced apart from the other. Although fiber Bragg gratings 1474, 1476 are shown as being identical in the non-limiting example of FIG. 7B, it should be understood that the grating parameters associated with gratings 1474, 1476 may also be non-identical. A cladding material 1475 is disposed on and surrounds at least a portion of the optical fiber core 1472. As shown, the cladding material 1475 may at least partially cover the fiber Bragg gratings 1474, 1476 and the region therebetween. The cladding material 1475 has at least one material property associated therewith, where the at least one material property may be a smaller Young's modulus than a Young's modulus of the optical fiber core 12, a larger photo-elastic coefficient than a photo-elastic coefficient of the optical fiber core 12, a lower refractive index (RI) than a refractive index of the optical fiber core 12, or combinations thereof. In embodiments, materials of the fiber Bragg grating may also be selected based on their thermal coefficient. It should be understood that the optical fiber core 1472 may be any suitable type of optical fiber core, such as those made from silica, silicon, optically transparent polymers, or the like. As a non-limiting example, if the optical fiber core 1472 is made from silica (SiO₂), the cladding material 1475 may be MY-133, a low refractive index optical coating manufactured by MY Polymers Ltd. of Israel, or BIO-133, also a low refractive index optical coating manufactured by MY Polymers Ltd. of Israel. As a further non-limiting example, if the core is silicon, which has a higher RI than silica, the cladding material 1475 may be polyvinylidene fluoride (PVDF), polystyrene (PS), parylene, benzocyclobutene (BCB), MY-133, or BIO-133. It should be further understood that, as an alternative, one of the fiber Bragg gratings 16, 18 may be replaced by any other suitable type of reflector, such as, for example, a metal coating mirror, a dielectric coating mirror, a total internal reflection mirror, or the like.

In the embodiment of FIG. 7A, the optical sensor structure 1421 (which may be any fiber end sensor discussed herein) may be employed for detection of acoustic signals as well as temperature and pressure measurements, as discussed herein. The fiber Bragg Grating 1470, which may include multiple fiber Bragg gratings 1470 arranged along a length of the fiber optic sensor may also be configured for temperature, pressure, and acoustic signal measurement. This arrangement permits the fiber optic sensor device to generate measurements of these parameters along its entire length, thus allowing for mapping of temperature and pressure.

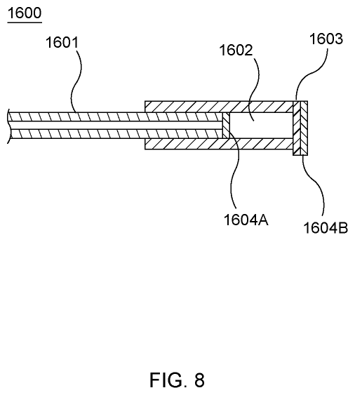

FIG. 8 illustrates a membrane based fiber optic sensor device. The membrane based fiber optic sensor device 1600 includes a fiber optic waveguide 1601, an air cavity 1602, a membrane 1603, and one or more reflective surfaces (e.g., mirrors) 1604A and 1604B. The membrane based fiber optic sensor device 1600 may be configured to provide high pressure sensitivity. The flexible membrane 1603 may deform when subject to external pressure, causing a distortion in the distal mirror 1604B and changes in the air cavity 1602 (e.g., in the length of the cavity). These distortions and changes may alter an incoming optical signal that is reflected by the distal mirror 1604B within the chamber created by the air cavity 1602. The changes to the optical signal may be present in a reflected optical signal, which may then be interpreted by an optical sensor system (e.g., as described above) to determine pressure. The membrane based fiber optical device 1600 may also be thermally sensitive and acoustically sensitive and therefore may operate in multiple modalities.

As discussed above, the various optical sensor structures may each be used in a multi-modal fashion to measure pressure, temperature, and incoming acoustic signals. Some of the above-described optical sensor structures may be particularly advantageous in measuring specific parameters. Advantageous qualities described in association with one or more of the sensor types described above does not imply any insufficiency in measurement capabilities of any other sensor type. For example, as described above, fiber-end sensors may be particularly advantageous in measuring ultrasound signals due their compact size, good acoustic bandwidth, omnidirectional receiving angles, and high bandwidth. In another example, FBG sensors may be particularly advantageous in measuring temperature, due to the large temperature range that can be measured, their distributed nature along the length of a fiber that creates a mapping capability, and ease of manufacturing. The membrane based fiber optical devices described herein may excel at pressure measurement, due to high sensitivity. The above described optical sensor structures may be combined in any suitable way to facilitate ablation procedures. Combinations of the described optical sensor structures may use some sensors in a unimodal fashion, e.g., collecting one type of data, and/or in a multimodal fashion, e.g., collecting multiple types of data by a single sensor.

The following describes several non-limiting examples of sensor combinations and structures consistent with the present disclosure.

In embodiments, any of the fiber end sensor devices described herein may be combined with an ultrasound emitter, such as a mechanical acoustic energy generating device (e.g., PZT), photo-acoustic effect generating device (e.g., from emitted laser light) to provide ultrasound imagery from a point-of-view of the device itself. Such a fiber end sensor may further be capable of temperature and pressure measurement as well as measurement of acoustic signals provided from another source. Such a combined device may further be combined with any of the distributed sensors described herein (e.g., FBGs or polarization based sensors) and disposed on the same fiber optic waveguide and/or any other fiber end sensor device described herein as an array.

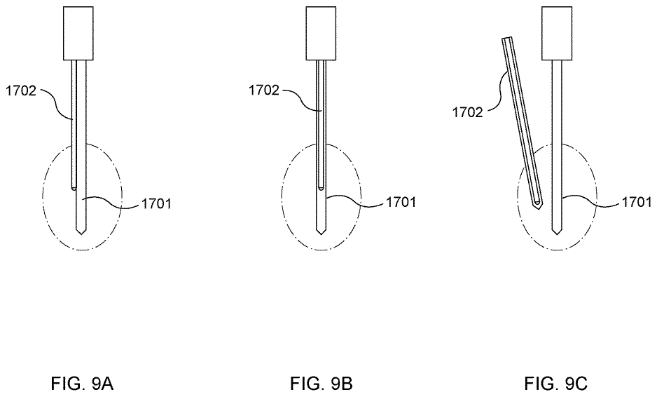

FIGS. 9A-9C illustrate various structural embodiments of fiber optic sensor devices consistent with the present disclosure. Each of FIGS. 9A-9C illustrate an ablation device 1701 and a fiber optic sensor device 1702. In FIG. 9A, the ablation device 1701 and the fiber optic sensor device 1702 are bundled or attached together such that the fiber optic sensor device 1702 is collocated with the ablation device 1701. In FIG. 9B, the fiber optic sensor device 1702 is integrated with and built directly into the ablation device 1701 to achieve collocation. In FIG. 9C, the ablation device 1701 and the fiber optic sensor device 1702 are maintained in separate structures. The fiber optic sensor device 1702 is packaged into a standalone probe. In embodiments, multiple fiber optic sensor device probes may be used with an ablation device 1701 during an ablation procedure.

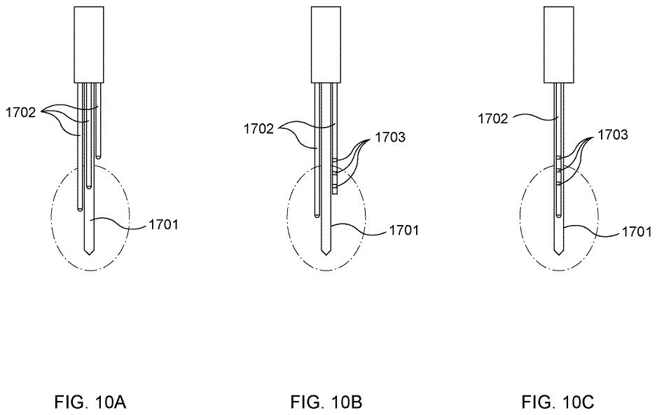

FIGS. 10A-10C illustrate various fiber optic sensor device combinations arranged structurally with an ablation device.

The embodiment of FIG. 10A includes a plurality of fiber optic sensor devices 1702 collocated with the ablation device 1701. The plurality of fiber optic sensor devices 1702 each include a fiber end sensor configured for temperature, pressure, and acoustic signal measurement. As illustrated in FIG. 10A, the plurality of fiber optic sensor devices 1702 are arranged such that their ends (e.g., the locations of the fiber end sensors) are located at different points along the length of the ablation device. This arrangement permits the fiber optic sensor devices 1702 to measure pressure, temperature, and acoustic signals at different locations, thus providing a mapping capability. As shown in FIG. 10A, each of the fiber optic sensor devices 1702 is arranged proximal to the ablation device tip. In embodiments, ablation energy may be provided from a vicinity of the tip and all fiber optic sensor devices 1702 are located proximal to the origin of ablation energy. In different embodiments, ablation energy may be provided from a location along the length of the ablation device 1701 and fiber optic sensor devices 1702 may be located both proximally and distally of the origin of ablation energy, thus providing the ability to map temperatures and pressures on both sides.

The embodiment of FIG. 10B includes a plurality of fiber optic sensor devices 1702 collocated with the ablation device 1701. At least one of the plurality of fiber optic sensor devices 1702 includes a fiber end sensor configured for temperature, pressure, and acoustic signal measurement. At least one of the plurality of fiber optic sensor devices 1702 includes a plurality of distributed optical sensors 1703 (e.g., multiple FBG sensors and/or multiple polarization based sensors) configured for temperature, pressure, and acoustic signal measurement and, optionally, a fiber end sensor. As illustrated in FIG. 10B, the plurality of fiber optic sensor devices 1702 are arranged such that the distributed optical sensors 1703 and/or any included fiber end sensors are arranged along the length of the ablation device 1702. This arrangement permits the fiber optic sensor devices 1702 and/or distributed optical sensors 1703 to measure pressure, temperature, and acoustic signals at different locations, thus providing a mapping capability. As shown in FIG. 10B, each of the fiber optic sensor devices 1702 and distributed optical sensors 1703 are arranged proximal to the ablation device tip. In embodiments, ablation energy may be provided from a vicinity of the tip and all fiber optic sensor devices 1702 and distributed optical sensors 1703 are located proximal to the origin of ablation energy. In different embodiments, ablation energy may be provided from a location along the length of the ablation device 1701 and fiber optic sensor devices 1702 and distributed optical sensors 1703 may be located both proximally and distally of the origin of ablation energy, thus providing the ability to map temperatures and pressures on both sides.

The embodiment of FIG. 10C includes a single fiber optic sensor device 1702 collocated with the ablation device 1701. The fiber optic sensor device 1702 includes a fiber end sensor and a plurality of distributed optical sensors 1703 (e.g., multiple FBG sensors and/or multiple polarization based sensors) configured for temperature, pressure, and acoustic signal measurement and, optionally, a fiber end sensor. In further embodiments, multiple fiber optic sensor devices 1702 having fiber end sensors and distributed optical sensors 1703 may be included. As illustrated in FIG. 10C, the fiber optic sensor device 1702 is arranged such the distributed optical sensors 1703 and/or any included fiber end sensors are arranged along the length of the ablation device 1702. This arrangement permits the fiber optic sensor devices 1702 and distributed optical sensors 1703 to measure pressure, temperature, and acoustic signals at different locations, thus providing a mapping capability. As shown in FIG. 10C, each of the fiber optic sensor devices 1702 and distributed optical sensors 1703 are arranged proximal to the ablation device tip. In embodiments, ablation energy may be provided from a vicinity of the tip and all fiber optic sensor devices 1702 and distributed optical sensors 1703 are located proximal to the origin of ablation energy. In different embodiments, ablation energy may be provided from a location along the length of the ablation device 1701 and fiber optic sensor devices 1702 and distributed optical sensors 1703 may be located both proximally and distally of the origin of ablation energy, thus providing the ability to map temperatures and pressures on both sides.

FIGS. 10A-10C illustrate embodiments of fiber optic sensor devices 1702 arranged with rigid ablation devices 1701. In embodiments, ablation devices may be flexible (e.g., based on catheters, guidewires, or other flexible delivery systems). In such embodiments, the fiber optic sensor devices 1702 may be likewise flexible.

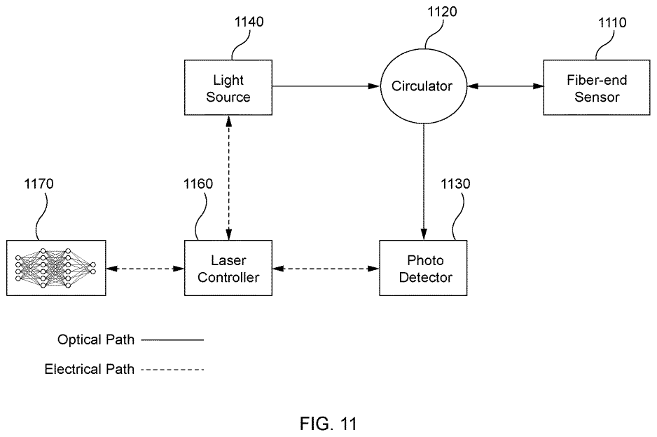

FIG. 11 illustrates an example optical sensing configuration for use with a fiber-end optical sensor device consistent with embodiments described herein. As shown in FIG. 11, the optical sensing system may include a light source 1140, a laser controller 1160, an optical circulator 1120, a fiber-end sensor 1110, and a photo detector 1130. Optical connections between optical components are illustrated as optical paths, while control or signal connections between electronic components are illustrated as electrical paths. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

In some embodiments, the laser controller 1160 controls operation of the light source 1140, which may comprise a coherent optical source such as a laser. In other embodiments, the light source may comprise a broadband or partially coherent optical source, such as a light-emitting diode (LED), a superluminescent diode (SLD), an amplified spontaneous emission (ASE) source, a tungsten-halogen lamp, an arc lamp, a supercontinuum source, or another suitable optical source capable of generating optical signals for interrogation of the fiber-optic sensor device, including an optical system on a chip. The light source 1140 generates an optical signal that is directed along the optical path to the optical circulator 1120. The optical circulator 1120 directs the optical signal toward the fiber-end sensor 1110, which may be disposed at a distal end of an optical waveguide or sensor fiber.

The fiber-end sensor 1110 receives the optical signal and interacts with the optical signal in a manner that depends on environmental conditions present at the sensor location. In various embodiments, the fiber-end sensor 1110 may include an optical sensing structure configured to respond to environmental parameters such as temperature, pressure, and/or acoustic signals, including ultrasound signals. Interaction between the optical signal and the sensing structure produces a returned optical signal that carries information representative of the sensed environmental conditions.

In some embodiments, the fiber-end sensor 1110 may include an optical resonator structure, such as a Fabry-Perot resonator, optical cavity resonator, whispering-gallery-mode resonator, micro-ring resonator, photonic crystal resonator, or any of the sensors described above with reference to FIGS. 1-10C above. Environmental temperature, pressure, and/or incident acoustic waves may induce changes in the resonator geometry, refractive index, or optical path length, thereby modifying the resonance condition of the resonator. These changes may produce detectable variations in the returned optical signal, such as shifts in phase, intensity, wavelength, or spectral characteristics.

In other embodiments, the fiber-end sensor 1110 may include an interferometer-based sensing structure. For example, the sensing structure may comprise a Mach-Zehnder interferometer, Michelson interferometer, Fabry-Perot interferometer, Sagnac interferometer, or other interferometric configuration. Environmental parameters may produce mechanical deformation, refractive index changes, or stress-induced optical effects within the sensing structure, thereby altering the optical phase relationship between interfering optical paths. The resulting interference changes may be detected in the returned optical signal.

In further embodiments, the fiber-end sensor 1110 may include a fiber Bragg grating sensor or other wavelength-selective optical structure. Environmental conditions such as temperature, pressure, or acoustic signals may alter the effective grating period or refractive index of the grating structure of the fiber Bragg grating, thereby shifting the reflected wavelength or modifying the spectral response of the grating. These changes may likewise be detected in the returned optical signal.

Regardless of the specific sensing architecture employed, the returned optical signal propagates back through the optical circulator 1120, which directs the returned signal toward the photo detector 1130. The photo detector 1130 converts the optical signal into an electrical signal representative of the sensed environmental conditions at the fiber-end sensor 1110.

The resulting electrical signal may then be processed by associated processing electronics or control systems, not shown in FIG. 11, to determine environmental parameters such as temperature, pressure, acoustic signals, or combinations thereof at the sensing location. In this manner, the configuration shown in FIG. 11 provides an optical interrogation architecture capable of supporting multiple types of fiber-optic sensing mechanisms while maintaining a simplified optical path between the light source, sensor, and detection components.

In some embodiments, one or more operations of the optical sensing system of FIG. 11, including temperature determination, acoustic sensing and/or imaging, and control of the ablation device, may be performed using one or more machine learning models 1170. The machine learning models may be configured to process optical signals corresponding to sensed environmental parameters and acoustic signals to generate outputs including temperature estimates, acoustic images, and/or control signals. Further details regarding such machine learning models and their integration with sensing and control operations are described with reference to FIG. 19.

In some embodiments, temperature sensing may also be performed using the fiber-end sensor 1110 disposed at the distal end of the optical path. The fiber-end sensor 1110 may include an optical sensing structure, such as a resonator, interferometric structure, fiber Bragg grating, or acoustically and thermally responsive polymer region, configured to translate temperature changes into measurable variations in an optical signal provided by the light source 1140 and routed through the circulator 1120. The laser controller 1160 controls the light source 1140 to deliver optical signals to the fiber-end sensor 1110, and the fiber-end sensor 1110 interacts with the optical signal and returns a modified optical signal through the circulator 1120 toward the photo detector 1130. Changes in the surrounding temperature may induce thermal expansion or contraction of the sensing structure and/or modify the refractive index of one or more materials in the sensor region through the thermo-optic effect. These changes may alter the effective optical path length, resonance condition, interference phase, or reflected wavelength associated with the fiber-end sensor 1110. As a result, the returned optical signal received at the photo detector 1130 may exhibit detectable changes in phase, intensity, wavelength, or spectral characteristics that correspond to the local temperature at the fiber-end sensor location. The fiber-end sensor 1110 may provide localized temperature measurements, which may be useful during procedures such as tissue ablation where monitoring the thermal environment near the distal end of a probe or delivery device is desirable.

In some embodiments, the optical sensing system of FIG. 11 is further configured to sense acoustic waves, including ultrasound waves, present in the environment surrounding the fiber-end sensor 1110. Acoustic waves incident on the fiber-end sensor 1110 may induce mechanical deformation and/or refractive index changes in the sensor structure, thereby modulating the optical signal transmitted through and returned by the optical fiber. The modulated optical signal may be detected by the photo detector 1130 and processed to determine characteristics of the acoustic waves for purposes including imaging, tracking, and/or guidance.

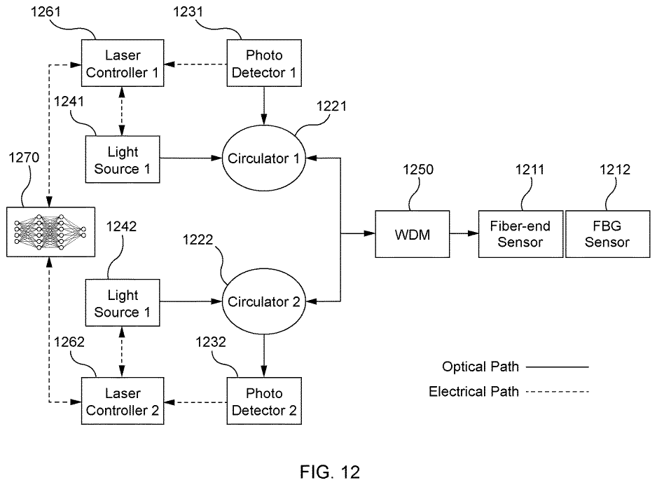

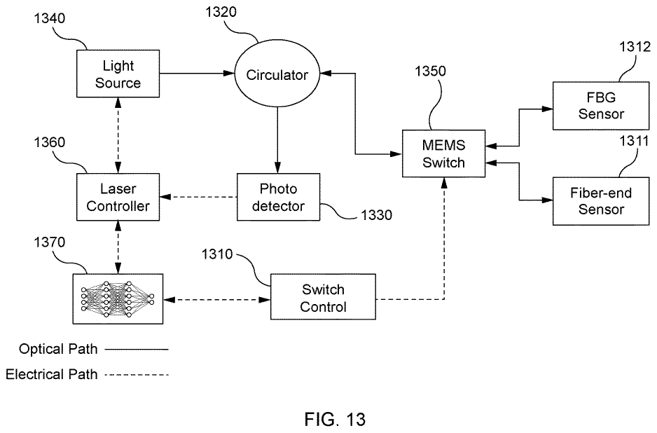

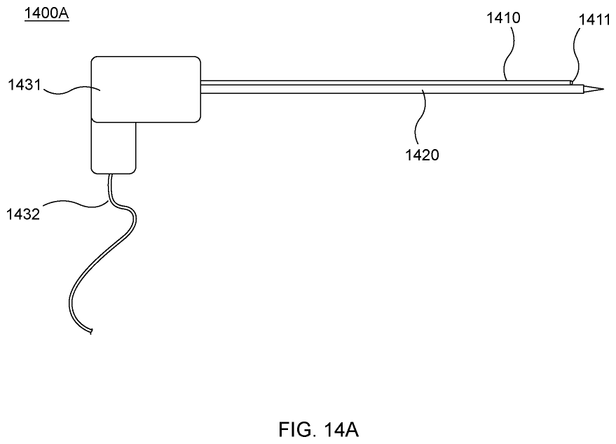

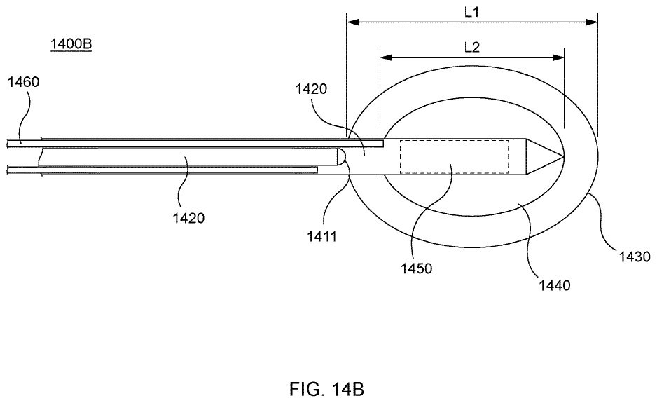

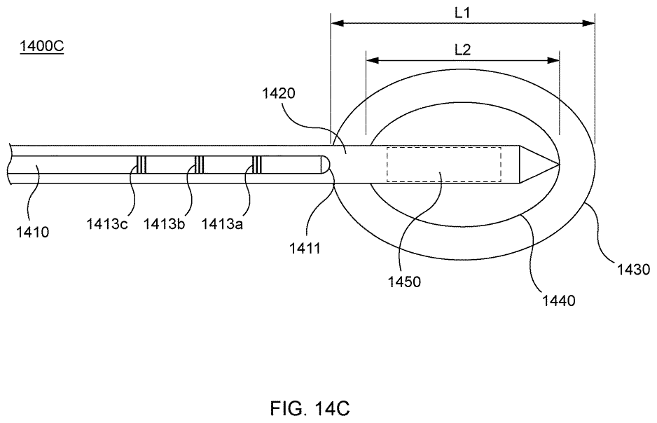

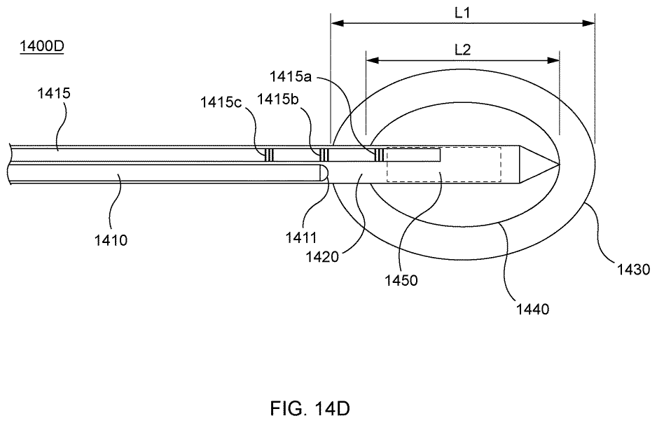

The configuration illustrated in FIG. 11 is provided by way of example and is not intended to be limiting. Other optical interrogation architectures may be used, such as, for example, the architectures illustrated in FIGS. 12-13, which are described below. In some examples, additional optical components such as splitters, couplers, wavelength-division multiplexing devices, polarization controllers, polarization analyzers, frequency shifters, optical filters, or additional circulators may be incorporated into the optical path. In some embodiments, multiple sensors may be arranged along a single optical fiber or across a plurality of fibers and interrogated using wavelength, time, frequency, or spatial multiplexing techniques. Accordingly, the architecture shown in FIG. 11 should be understood as one example of an optical interrogation system capable of interacting with fiber-optic sensor devices described herein such as, for example, the fiber optic sensor device 1410 illustrated in FIG. 14A.

FIG. 12 illustrates another example optical sensing configuration for use with fiber-optic sensor devices consistent with embodiments described herein. As shown in FIG. 12, the optical sensing system may include a first light source 1241 and a second light source 1242, corresponding laser controllers 1261 and 1262, photo detectors 1231 and 1232, circulators 1221 and 1222, a wavelength-division multiplexing (WDM) device 1250, a Fiber Bragg grating (FBG) sensor 1212, and a fiber-end sensor 1211. Optical connections between optical components are illustrated as optical paths, while control or signal connections between electronic components are illustrated as electrical paths. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

In some embodiments, as illustrated in FIG. 12, the optical sensing configuration may include two separate sensing channels that operate independently to interrogate different sensor types. A first sensing channel may include the first light source 1241, the laser controller 1261, the circulator 1221, and the photo detector 1231, and may be configured to interrogate the FBG sensor 1212. A second sensing channel may include the second light source 1242, the laser controller 1262, the circulator 1222, and the photo detector 1232, and may be configured to interrogate the fiber-end sensor 1211. In some embodiments, the wavelength-division multiplexing (WDM) device 1250 may be used to combine and/or separate optical signals associated with the two sensing channels within the optical path. In this manner, the two sensing channels may operate independently or in parallel, allowing the system to monitor environmental parameters such as temperature, pressure, or acoustic signals using either sensor type or both simultaneously.

In the configuration of FIG. 12, the optical sensing system may employ wavelength division multiplexing to interrogate multiple sensing elements along a common optical fiber. In particular, a plurality of optical wavelengths λ₁, . . . , λᵢ may be provided to a corresponding plurality of fiber Bragg grating (FBG) sensors disposed along the optical fiber. Each FBG sensor may be configured to reflect a respective wavelength band centered at one of λ₁, . . . , λᵢ, thereby enabling independent interrogation of multiple sensing locations using a single optical waveguide.

In addition to the FBG sensors, the system may include a fiber-end sensor configured to operate at a wavelength λₚ. The wavelength λₚ may be distinct from the wavelengths λ₁, . . . , λᵢ associated with the FBG sensors, thereby allowing the fiber-end sensor to be interrogated concurrently with the FBG sensors without spectral overlap. In this manner, the optical sensing configuration of FIG. 12 may simultaneously obtain distributed sensing data from the FBG sensors and localized sensing data from the fiber-end sensor using a multiplexed optical signal. Environmental parameters such as temperature, pressure, and/or acoustic signals may induce shifts in the reflected wavelengths of the FBG sensors and/or changes in the optical response at wavelength λₚ of the fiber-end sensor. These wavelength shifts and/or signal modulations may be detected and processed to determine localized environmental conditions along the optical fiber.

In some embodiments, the laser controllers 1261 and 1262 may control operation of the light sources 1241 and 1242, respectively. The light sources may comprise coherent optical sources such as lasers. In other embodiments, the light sources may comprise broadband or partially coherent optical sources, such as light-emitting diodes (LEDs), superluminescent diodes (SLDs), amplified spontaneous emission (ASE) sources, tungsten-halogen lamps, arc lamps, supercontinuum sources, or other suitable optical sources capable of generating optical signals for interrogation of fiber-optic sensor devices. The first light source 1241 may generate a first optical signal and the second light source 1242 may generate a second optical signal, which may be introduced into respective optical paths through the circulators 1221 and 1222.

In some embodiments, the wavelength-division WDM device 1250 is configured to combine and/or separate optical signals having different wavelengths. For example, optical signals associated with the first light source 1241 and the second light source 1242 may be multiplexed by the WDM device 1250 into a shared optical path and may thereafter be directed toward one or more sensing locations. Returned optical signals may likewise be de-multiplexed by the WDM device 1250 and routed into corresponding detection paths. In this manner, the WDM device 1250 may enable interrogation of multiple sensors using different wavelengths within the same optical sensing architecture.

The optical sensing system of FIG. 12 may include both the fiber-end sensor 1211 and the FBG sensor 1212. In some embodiments, the multiplexed or distributed optical signals may be directed toward the fiber-end sensor 1211 and the FBG sensor 1212, each of which may interact with incident optical signals in a manner that depends on environmental conditions present at the sensor location. In various embodiments, the fiber-end sensor 1211 and the FBG sensor 1212 may be configured to respond to environmental parameters such as temperature, pressure, and/or acoustic signals, including ultrasound signals. Interaction between the optical signals and the sensing structures may produce returned optical signals carrying information representative of the sensed environmental conditions.

In some embodiments, the optical sensing configuration of FIG. 12 may be used in an operating environment in which different sensor types are assigned different sensing functions. For example, the fiber-end sensor 1211 may be configured for acoustic sensing, such as ultrasound sensing, and may be used to detect acoustic signals for guidance, tracking, and image generation, while the FBG sensor 1212 may be configured for temperature sensing. The FBG sensor 1212 may generate temperature-related data in the vicinity of the FBG sensor 1212. In such an embodiment, a first sensing channel including the first light source 1241, the laser controller 1261, the circulator 1221, and the photo detector 1231 may be used to interrogate the fiber-end sensor 1211, and a second sensing channel including the second light source 1242, the laser controller 1262, the circulator 1222, and the photo detector 1232 may be used to interrogate the FBG sensor 1212. The WMD device 1250 may be used to combine and/or separate optical signals associated with the two sensing channels.

In an operating environment such as a tissue ablation procedure, the fiber-end sensor 1211 may be positioned on or near a distal end of an ablation probe, catheter, needle, or other delivery device and may detect acoustic signals that are used to generate image data representative of surrounding tissue and/or the position of the device within the tissue. In some embodiments, the fiber-end sensor 1211 may detect transmitted acoustic waves, reflected acoustic waves, scattered acoustic waves, tissue harmonics, or combinations thereof, and the detected signals may be processed to generate image data useful for device guidance and monitoring of the treatment region. At the same time, the FBG sensor 1212 may sense temperature in the local environment by translating thermally induced changes in grating period and/or refractive index into shifts in reflected wavelength or other detectable spectral changes. Such temperature measurements may be useful during procedures involving heating or cooling of tissue, including thermal ablation techniques such as radiofrequency, microwave, laser, or ultrasound ablation, as well as cooling-based procedures such as cryoablation. In this manner, the fiber-end sensor 1211 may provide acoustic sensing and imaging functionality, while the FBG sensor 1212 may provide localized temperature monitoring, thereby enabling simultaneous imaging and thermal monitoring during therapeutic procedures.

In some embodiments, energy that is delivered by an ablation device, such as one of the ablation devices illustrated in the examples of FIGS. 14A-14D, may produce a central ablation zone surrounded by a thermal exposure zone within the surrounding tissue. The ablation zone may correspond to a region in which tissue temperatures reach levels sufficient to produce rapid and irreversible tissue damage, such as coagulative necrosis. For many heat-based ablation modalities, including radiofrequency ablation, microwave ablation, laser ablation, and high-intensity focused ultrasound ablation, temperatures within the ablation zone may typically exceed approximately 60° C., and may in some cases reach 80°-100° C. or higher near the energy delivery source. At temperatures above approximately 50°−55° C., rapid protein denaturation and cellular injury may occur, and temperatures above approximately 60° C. may result in near-instantaneous cell death. In some cases, temperatures exceeding 100° C. may lead to tissue vaporization or boiling near the ablation device.

Surrounding the ablation zone, the tissue may experience a thermal exposure zone, in which tissue temperatures are elevated above normal physiological temperature, such as approximately 37° C., but remain below the threshold typically associated with immediate tissue necrosis. For example, tissue within the thermal exposure zone may experience temperatures in the range of approximately 40°−60° C. during heat-based ablation procedures. Within this range, thermal exposure may cause reversible cellular stress, delayed tissue injury, or sub-lethal thermal effects depending on the temperature level and duration of exposure. In some embodiments, the fiber-optic sensing systems described herein may be used to monitor these temperature distributions during therapeutic procedures. For example, the FBG sensor 1212 may detect temperature within the thermal exposure zone or the ablation zone to estimate proximity to the ablation zone, while the fiber-end sensor 1211 may remain positioned outside the most extreme temperature region while performing acoustic sensing or imaging functions. In this manner, monitoring temperatures within the thermal exposure zone may assist in guiding device positioning, estimating the size and location of the ablation zone, and reducing unintended thermal injury to surrounding tissue.

In some embodiments, the fiber-end sensor 1211 may include an optical resonator structure, such as a Fabry-Perot resonator, optical cavity resonator, whispering-gallery-mode resonator, micro-ring resonator, photonic crystal resonator, or any of the sensors described above with reference to FIGS. 1-11. Environmental temperature, pressure, and/or incident acoustic waves may induce changes in resonator geometry, refractive index, or optical path length, thereby modifying the resonance condition of the resonator. These changes may produce detectable variations in the returned optical signal, such as shifts in phase, intensity, wavelength, or spectral characteristics.

In other embodiments, the fiber-end sensor 1211 may include an interferometer-based sensing structure. For example, the sensing structure may comprise a Mach-Zehnder interferometer, Michelson interferometer, Fabry-Perot interferometer, Sagnac interferometer, or other interferometric configuration. Environmental parameters may produce mechanical deformation, refractive index changes, or stress-induced optical effects within the sensing structure, thereby altering the optical phase relationship between interfering optical paths. The resulting interference changes may be detected in the returned optical signal.

In some embodiments, the FBG sensor 1212 may comprise a fiber Bragg grating or other wavelength-selective optical structure. Environmental conditions such as temperature, pressure, or acoustic signals may alter the effective grating period and/or refractive index of the grating structure, thereby shifting the reflected wavelength or modifying the spectral response of the grating. These changes may be detected in the returned optical signal and may be used to determine one or more sensed environmental conditions.

Returned optical signals from the fiber-end sensor 1211 and the FBG sensor 1212 may propagate back through the optical sensing architecture toward the WDM device 1250, which may separate the returned signals according to wavelength and route the signals through circulators 1221 and 1222 toward the corresponding photo detectors 1231 and 1232. The photo detectors 1231 and 1232 convert the returned optical signals into electrical signals representative of the sensed environmental conditions at the corresponding sensor locations.

The electrical signals produced by the photo detectors 1231 and 1232 may then be processed by associated processing electronics or control systems, not shown in FIG. 12, to determine environmental parameters such as temperature, pressure, acoustic signals, or combinations thereof. By using multiple light sources and a wavelength-division multiplexing architecture, the configuration shown in FIG. 12 may enable interrogation of multiple fiber-optic sensor devices and may support sensing of different parameters at different sensing locations within the same system.

In some embodiments, one or more operations of the optical sensing system of FIG. 12, including distributed temperature sensing using FBG sensors, acoustic sensing and/or imaging using the fiber-end sensor, and control of the ablation device, may be performed using one or more machine learning models 1270. The machine learning models may process multi-wavelength optical signals obtained via wavelength division multiplexing to extract features associated with temperature and acoustic responses and to generate outputs including temperature profiles, acoustic images, and/or control signals. Further details regarding such machine learning models are described with reference to FIG. 19.

In some embodiments, temperature sensing may be performed using one or both of the fiber-end sensor 1211 and the FBG sensor 1212. The fiber-end sensor 1211 may include an optical sensing structure configured to translate temperature changes into measurable variations in an optical signal provided by one or both of the light sources 1241, 1242 and routed through one or both of the circulators 1221, 1222. Similarly, the FBG sensor 1212 may translate temperature changes into shifts in reflected wavelength or other spectral characteristics. Changes in the surrounding temperature may induce thermal expansion or contraction of the sensing structures and/or modify the refractive index of one or more materials in the sensor regions through the thermo-optic effect. These changes may alter effective optical path length, resonance condition, interference phase, grating period, or reflected wavelength associated with the sensors 1211 and 1212. As a result, the returned optical signals received at the photo detectors 1231 and 1232 may exhibit detectable changes corresponding to the local temperature at the sensor locations. The sensors 1211 and 1212 may thereby provide localized temperature measurements, which may be useful during procedures such as tissue ablation where monitoring the thermal environment near the distal end of a probe or delivery device is desirable.

In some embodiments, the optical sensing system of FIG. 12 is further configured to sense acoustic waves, including ultrasound waves, present in the environment surrounding the fiber-end sensor 1211. Acoustic waves incident on the fiber-end sensor 1211 may cause changes in optical properties of the sensor, including deformation and/or refractive index variation, resulting in modulation of the reflected optical signals associated with the fiber-end sensor 1211. In embodiments employing wavelength division multiplexing, such acoustic-induced modulation may be encoded in one or more wavelength channels and detected by the corresponding photodetectors (e.g., 1231, 1232), enabling processing of acoustic information for imaging, tracking, and/or guidance.

The optical sensing configuration illustrated in FIG. 12 is provided by way of example and is not intended to be limiting. Other multiplexed optical interrogation architectures may be used consistent with the principles described herein. In some examples, additional optical components such as splitters, couplers, wavelength-division multiplexing devices, polarization controllers, polarization analyzers, frequency shifters, optical filters, or additional circulators may be incorporated into the optical path. In some embodiments, multiple sensors may be arranged along a single optical fiber or across a plurality of fibers and interrogated using wavelength, time, frequency, or spatial multiplexing techniques. Accordingly, the architecture shown in FIG. 12 should be understood as one example of an optical interrogation system capable of interacting with fiber-optic sensor devices described herein such as, for example, the ablation device 1400C illustrated in FIG. 14C.

FIG. 13 illustrates another example optical sensing configuration for use with fiber-optic sensor devices consistent with embodiments described herein. As shown in FIG. 13, the optical sensing system may include a light source 1340, a laser controller 1360, an optical circulator 1320, a photo detector 1330, a MEMS switch 1350, a switch control 1310, an FBG sensor 1312, and a fiber-end sensor 1311. Optical connections between optical components are illustrated as optical paths, while control or signal connections between electronic components are illustrated as electrical paths. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

In some embodiments, the laser controller 1360 controls operation of the light source 1340, which may comprise a coherent optical source such as a laser. In other embodiments, the light source may comprise a broadband or partially coherent optical source, such as a light-emitting diode (LED), a superluminescent diode (SLD), an amplified spontaneous emission (ASE) source, a tungsten-halogen lamp, an arc lamp, a supercontinuum source, or another suitable optical source capable of generating optical signals for interrogation of fiber-optic sensor devices. The light source 1340 generates an optical signal that is directed along the optical path to the optical circulator 1320.

In some embodiments, the switch control 1310 is configured to control operation of the MEMS switch 1350. The switch control 1310 may provide electrical control signals that cause the MEMS switch 1350 to select a desired optical path. For example, the switch control 1310 may cause the MEMS switch 1350 to couple the optical path from the circulator 1320 to the fiber-end sensor 1311 during a first sensing interval and to the FBG sensor 1312 during a second sensing interval. In this manner, the optical sensing system may selectively interrogate different sensors in a time-multiplexed manner.

The MEMS switch 1350 may comprise a micro-electro-mechanical systems (MEMS) optical switch configured to selectively route optical signals between different optical paths using micro-scale mechanically actuated switching elements. In some embodiments, the MEMS switch 1350 may include one or more movable micro-mirrors, cantilever structures, or other micro-mechanical optical elements that redirect an optical beam between multiple optical fibers or waveguides.

In some embodiments, the MEMS switch 1350 may be actuated using electrostatic, thermal, piezoelectric, or electromagnetic actuation mechanisms. Actuation of the MEMS switch 1350 may be controlled by the switch control 1310, which may apply control signals to position the movable optical elements so that the optical signal is directed toward a selected sensor path. The MEMS switch 1350 may enable a single interrogation light source to be selectively coupled to multiple sensors, such as the FBG sensor 1312 and the fiber-end sensor 1311. Thus, the MEMS switch 1350 may support time-multiplexed interrogation of multiple sensing elements while allowing the optical sensing system to share a common light source and detection path.

The fiber-end sensor 1311 and the FBG sensor 1312 may each interact with incident optical signals in a manner that depends on environmental conditions present at the sensor location. In various embodiments, the fiber-end sensor 1311 and the FBG sensor 1312 may be configured to respond to environmental parameters such as temperature, pressure, and/or acoustic signals, including ultrasound signals. Interaction between the optical signal and the sensing structure may produce a returned optical signal carrying information representative of the sensed environmental conditions.

In some embodiments, the fiber-end sensor 1311 may include an optical resonator structure, such as a Fabry-Perot resonator, optical cavity resonator, whispering-gallery-mode resonator, micro-ring resonator, photonic crystal resonator, or any of the sensors described above with reference to FIGS. 1-12. Environmental temperature, pressure, and/or incident acoustic waves may induce changes in the resonator geometry, refractive index, or optical path length, thereby modifying the resonance condition of the resonator. These changes may produce detectable variations in the returned optical signal, such as shifts in phase, intensity, wavelength, or spectral characteristics.

In some embodiments, the fiber-end sensor 1311 may include an interferometer-based sensing structure. For example, the sensing structure may comprise a Mach-Zehnder interferometer, Michelson interferometer, Fabry-Perot interferometer, Sagnac interferometer, or other interferometric configuration. Environmental parameters may produce mechanical deformation, refractive index changes, or stress-induced optical effects within the sensing structure, thereby altering the optical phase relationship between interfering optical paths. The resulting interference changes may be detected in the returned optical signal.

In some embodiments, the FBG sensor 1312 may comprise a fiber Bragg grating or other wavelength-selective optical structure. Environmental conditions such as temperature, pressure, or acoustic signals may alter the effective grating period or refractive index of the grating structure, thereby shifting the reflected wavelength or modifying the spectral response of the grating. These changes may be detected in the returned optical signal and may be used to determine one or more sensed environmental conditions.

Returned optical signals from the selected sensor, such as the fiber-end sensor 1311 or the FBG sensor 1312, may propagate back through the MEMS switch 1350 and the optical circulator 1320, which directs the returned signal toward the photo detector 1330. The photo detector 1330 converts the optical signal into an electrical signal representative of the sensed environmental conditions at the selected sensor location.

The electrical signal produced by the photo detector 1330 may then be processed by associated processing electronics or control systems, not shown in FIG. 13, to determine environmental parameters such as temperature, pressure, acoustic signals, or combinations thereof. In this manner, the configuration shown in FIG. 13 may enable selective interrogation of multiple fiber-optic sensor devices using a shared light source, circulator, and detection path.

In some embodiments, one or more operations of the optical sensing system of FIG. 13, including temperature sensing, acoustic sensing and/or imaging, and control of the ablation device, may be performed using one or more machine learning models 1370. The machine learning models may process optical signals obtained from selectively interrogated sensors, for example via the MEMS switch, to generate outputs including temperature estimates, acoustic images, and/or control signals for operation of the ablation device. Further details regarding such machine learning models and associated processing are described with reference to FIG. 19.

In some embodiments, the optical sensing system of FIG. 13 is further configured to sense acoustic waves, including ultrasound waves, present in the environment surrounding the fiber-end sensor 1311. Acoustic waves incident on the fiber-end sensor 1311 may induce mechanical and/or optical property changes in the sensor structure, resulting in modulation of the optical signal returned through the optical fiber. In embodiments utilizing the MEMS switch 1350, optical interrogation of the fiber-end sensor 1311 may be selectively controlled, and the resulting modulated optical signals may be detected by the photo detector 1330 and processed to determine acoustic characteristics for imaging, tracking, and/or guidance.

In some embodiments, the selectable architecture shown in FIG. 13 may be used in an operating environment in which different sensor types are assigned different sensing functions. For example, the fiber-end sensor 1311 may be configured for acoustic sensing and imaging, while the FBG sensor 1312 may be configured for temperature sensing. The switch control 1310 may control the MEMS switch 1350 to selectively interrogate the sensors during different sensing intervals. Such an arrangement may be useful during procedures such as tissue ablation, in which acoustic imaging and temperature monitoring may both be desirable.

The configuration illustrated in FIG. 13 is provided by way of example and is not intended to be limiting. Other switched optical interrogation architectures may be used consistent with the principles described herein. In some examples, additional optical components such as splitters, couplers, wavelength-division multiplexing devices, polarization controllers, polarization analyzers, frequency shifters, optical filters, additional circulators, or additional switching elements may be incorporated into the optical path. Accordingly, the architecture shown in FIG. 13 should be understood as one example of an optical interrogation system capable of interacting with fiber-optic sensor devices described herein such as, for example, the ablation device 1400B illustrated in FIG. 14B.

FIGS. 11-13 illustrate some examples of optical interrogation architectures for use with the fiber-optic sensor devices described herein, such as the ablation devices 1400A, 1400B, and 1400C illustrated in FIGS. 14A-14C, respectively. FIG. 11 illustrates a single-sensor interrogation architecture in which a light source, circulator, and photo detector are used to interrogate a single fiber-optic sensor. FIG. 12 illustrates a parallel multi-sensor interrogation architecture in which multiple sensing channels and a wavelength-division multiplexing device may be used to interrogate multiple sensors, such as a fiber-end sensor and an FBG sensor, in parallel or substantially simultaneously. FIG. 13 illustrates a switched multi-sensor interrogation architecture in which a switching element, such as a MEMS switch, may be used to selectively route optical signals between different sensor paths so that multiple sensors may be interrogated using a shared optical source and detection path. In FIG. 13, the fiber-end sensor 1311 and the FBG sensor 1312 may be selectively interrogated through the switched optical path. Thus, the optical sensing systems described herein may be implemented in single-sensor, parallel multi-sensor, or switched multi-sensor configurations depending on the requirements of the particular sensing application.

FIG. 14A illustrates an example ablation device 1400A incorporating a fiber optic sensing configuration, consistent with embodiments described herein. As shown in FIG. 14A, the ablation device 1400A includes an ablation device body 1431, an ablation probe 1420 extending from the ablation device body 1431, and an electrical cable housing 1432 configured to provide electrical connectivity to the ablation device 1400A. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

The ablation device 1400A may be configured for use with a variety of ablation modalities, including heat-based ablation modalities and cold-based ablation modalities. For example, the ablation device 1400A may be configured to perform radiofrequency ablation, microwave ablation, laser ablation, ultrasound ablation, resistive heating ablation, cryoablation, electroporation, or other suitable tissue treatment procedures. In some embodiments, the ablation device 1400A may be powered via the electrical cable housing 1432. In other embodiments, the ablation device 1400A may be powered by alternative power sources, including an internal or external battery, wireless power transfer, inductive coupling, or other suitable power delivery mechanisms.

A fiber optic sensor device 1410 is coupled to the ablation device 1400A and extends along at least a portion of the ablation probe 1420. The fiber optic sensor device 1410 may include an optical fiber and a fiber-end sensor 1411 disposed at a distal portion of the optical fiber. In some embodiments, one or more fiber Bragg grating (FBG) sensors may be disposed along a length of the optical fiber to enable distributed sensing along the ablation probe 1420. The fiber optic sensor device 1410 may be configured to transmit and receive optical signals for sensing one or more environmental parameters. In some embodiments, the electrical cable housing 1432 may house, route, and protect the optical fiber or other cable associated with the fiber optic sensor device 1410, as well as one or more additional cables, conductors, or communication lines associated with powering, controlling, or operating the ablation device 1400A and/or the fiber optic sensor device 1410. Various configurations and arrangements of the fiber optic sensor device 1410, including configurations employing additional sensing elements and/or additional fiber optic sensor devices, are described in further detail below with respect to FIGS. 14B-16D.

The fiber-end sensor 1411 may be positioned at or near a distal end of the ablation probe 1420 such that the fiber-end sensor 1411 is located proximate to a treatment region during use of the ablation device 1400A. In this manner, the fiber-end sensor 1411 may be configured to sense environmental conditions at or near a site of energy delivery.

The fiber optic sensor device 1410 may be optically coupled to an optical sensing system, such as the optical sensing configuration described with respect to any of FIGS. 11-13, to enable interrogation of the fiber-end sensor 1411. For example, optical signals may be transmitted along the optical fiber of the fiber optic sensor device 1410 to the fiber-end sensor 1411, and optical signals modified by environmental conditions at the fiber-end sensor 1411 may be returned along the optical fiber for detection and processing.

In some embodiments, the fiber optic sensor device 1410 may be configured to detect one or more environmental parameters, including temperature, pressure, and/or acoustic signals associated with operation of the ablation device 1400A. The detected parameters may be used to monitor the ablation procedure and to provide feedback for controlling operation of the ablation probe 1420.

Accordingly, the configuration of FIG. 14A provides an integrated ablation and sensing system in which the fiber optic sensor device 1410 is incorporated into the ablation device 1400A to enable real-time sensing of conditions at or near the treatment site. The sensing provided by the fiber optic sensor device 1410 may also improve guidance, positioning, and monitoring of the ablation probe 1420 during the procedure.

FIG. 14B illustrates an example of an ablation device 1400B incorporating a fiber optic sensing configuration for monitoring thermal characteristics of an ablation procedure, consistent with embodiments described herein. As shown in FIG. 14B, the ablation device 1400B includes an ablation probe 1420 configured to deliver energy from a heating source 1450 to a target region. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

The heating source 1450 may generate an ablation region defined by an ablation zone boundary 1440, within which tissue is subjected to temperatures sufficient to cause irreversible tissue damage. A surrounding region defined by a thermal exposure zone boundary 1430 may correspond to tissue exposed to elevated temperatures below an ablation threshold. The thermal exposure zone may have a diameter L1, and the ablation zone may have a diameter L2, where L1 is greater than L2. The ablation zone and the thermal exposure zone are discussed above with reference to FIG. 12, above.

A fiber optic sensor device 1410 is positioned relative to the ablation probe 1420 and may include a fiber-end sensor 1411 disposed at or near a distal portion of the optical fiber. The fiber-end sensor 1411 may be configured to sense environmental parameters at or near the ablation region, including temperature, pressure, and/or acoustic signals associated with operation of the heating source 1450.

In some embodiments, the fiber optic sensor device 1410 may be used to detect temperature gradients corresponding to the ablation zone boundary 1440 and the thermal exposure zone boundary 1430. For example, measurements obtained from the fiber-end sensor 1411 may be used to estimate the extent of the ablation zone and the surrounding thermal exposure zone, including determination of L2 and L1.

The ablation device 1400B may further include a thermocouple 1460 positioned within or near the ablation probe 1420. The thermocouple 1460 may be configured to provide a temperature measurement associated with the ablation region. In some embodiments, the thermocouple 1460 may be used to calibrate, validate, or supplement measurements obtained from the fiber optic sensor device 1410. In some embodiments, the thermocouple 1460 may be positioned within a bonding or potting region used to secure the fiber optic sensor device 1410 to the ablation probe 1420. For example, the thermocouple 1460 and the optical fiber may be co-located within a common adhesive, potting material, or protective sheath, such that the thermocouple 1460 is mechanically coupled to the ablation probe 1420 together with the fiber optic sensor device 1410.

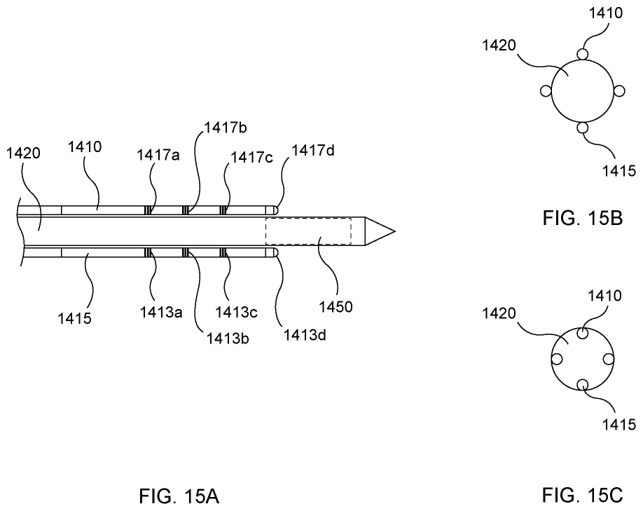

In some embodiments, the fiber optic sensor device 1410 may be secured to the ablation probe 1420 using one or more attachment techniques, including bonding, potting, coating, heat-shrink tubing, lamination, mechanical retention, or combinations thereof. Example embodiments for attaching the fiber optic sensor device 1410 to the ablation probe 1420 are described in further detail with reference to FIGS. 15A-15B.

In some embodiments, the fiber-end sensor 1411 may be any of the fiber optic sensor devices or fiber-end sensing structures disclosed in the present specification, such as, for example, the sensor fiber 301 including the optical resonator structure 321 of FIG. 3, the sensor fiber 351 including the optical resonator structure 361 and Fabry-Perot resonator 362 of FIG. 4, the interferometer-based fiber optic sensor device 101B including the fiber-end sensor structure 321A of FIGS. 5A and 5B, the fiber optic sensor device 101C including the optical sensor structure 329C of FIG. 5C, and the fiber optic sensor device 101D including the optical sensor structure 329D of FIG. 5D. In further embodiments, the fiber-end sensor 1411 may also be implemented using any of the additional fiber optic sensor device configurations disclosed in FIGS. 6A-8.

The fiber optic sensor device 1410 may be optically coupled to an optical sensing system, such as the configurations described with respect to FIGS. 11-13, to enable interrogation of the fiber-end sensor 1411. Optical signals transmitted along the optical fiber may be modulated by environmental conditions at the fiber-end sensor 1411, and the modulated signals may be detected and processed to determine parameters associated with the ablation procedure, such as temperature and pressure. In addition to sensing such parameters, the fiber-end sensor 1411 may also be used for imaging, including ultrasound-based imaging, guidance, tracking, and/or visualization of tissue and/or the position of the ablation probe 1420 during the procedure.

Accordingly, the ablation device 1400B of FIG. 14B enables real-time monitoring of thermal characteristics of the ablation procedure, including the extent of the ablation zone and the surrounding thermal exposure zone. The sensed information may be used to improve tracking, guidance, positioning, and control of the ablation probe 1420, and to reduce unintended thermal damage to surrounding tissue.

FIG. 14C illustrates an example ablation device 1400C incorporating a fiber optic sensing configuration with distributed sensing capability, consistent with embodiments described herein. As shown in FIG. 14C, the ablation device 1400C includes an ablation probe 1420 and a fiber optic sensor device 1410 extending along at least a portion of the ablation probe 1420. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

In the ablation device 1400C, the fiber optic sensor device 1410 may include an optical fiber having a plurality of fiber Bragg grating (FBG) sensors 1413a, 1413b, and 1413c disposed along a length thereof. The FBG sensors 1413a-1413c may be spaced along the optical fiber at predetermined locations to provide sensing at multiple positions along the ablation probe 1420.

Each of the FBG sensors 1413a-1413c may be configured to reflect a respective wavelength band and to exhibit a shift in the reflected wavelength in response to changes in environmental conditions, such as temperature. In some embodiments, the FBG sensors 1413a-1413c may be interrogated using wavelength division multiplexing, such that each FBG sensor is associated with a different wavelength, thereby enabling simultaneous interrogation of multiple sensing locations along the optical fiber.

Each of the FBG sensors 1413a-1413c may be configured to detect environmental parameters, including temperature, strain, pressure, and/or acoustic signals, at corresponding locations along the ablation probe 1420. In this manner, the fiber optic sensor device 1410 may provide spatially distributed sensing data along the ablation probe 1420, in contrast to single-point sensing configurations.

In some embodiments, measurements obtained from the FBG sensors 1413a-1413c may be used to determine a spatial profile of temperature along the ablation probe 1420 during operation. For example, the distributed sensing data may be used to estimate the extent of an ablation zone and a surrounding thermal exposure zone, and to monitor changes in these zones over time.

The fiber optic sensor device 1410 may further include a fiber-end sensor 1411 disposed at or near a distal end of the optical fiber. In some embodiments, the fiber-end sensor 1411 may be particularly suited for sensing acoustic signals and/or for imaging. For example, the fiber-end sensor 1411 may detect acoustic waves generated during operation of the ablation probe 1420, including signals associated with cavitation, tissue interaction, or energy deposition. The fiber-end sensor 1411 is described above with reference to FIG. 14B, and the above description of the fiber-end sensor 1411 is incorporated herein.

In some embodiments, the fiber-end sensor 1411 may be used for imaging, including ultrasound-based imaging, acousto-optic imaging, optoacoustic imaging, and/or other imaging modalities. The fiber-end sensor 1411 may thereby provide information regarding tissue structure, boundaries, and/or the position of the ablation probe 1420 relative to surrounding tissue.

The plurality of FBG sensors 1413a-1413c and the fiber-end sensor 1411 may be configured to provide complementary sensing. For example, the FBG sensors may provide distributed measurements of temperature, strain, and/or pressure along the ablation probe 1420, while the fiber-end sensor 1411 may provide localized acoustic sensing and/or imaging at or near the distal end of the ablation probe 1420.

The fiber optic sensor device 1410 may be optically coupled to an optical sensing system, such as the configurations described with respect to FIGS. 11-13, to enable interrogation of the FBG sensors 1413a-1413c and the fiber-end sensor 1411. Optical signals transmitted along the optical fiber may be reflected and/or modulated by the sensors in response to environmental conditions, and the resulting signals may be detected and processed.

Accordingly, the configuration of FIG. 14C provides an ablation device with both distributed sensing and localized acoustic and/or imaging capability, enabling enhanced tracking, monitoring, guidance, and control of the ablation probe 1420 during a procedure.

FIG. 14D illustrates an example ablation device 1400D incorporating multiple fiber optic sensor devices, consistent with embodiments described herein. As shown in FIG. 14D, the ablation device 1400D includes an ablation probe 1420, a first fiber optic sensor device 1410, and a second fiber optic sensor device 1415, each positioned relative to the ablation probe 1420. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

In the ablation device 1400D, the first fiber optic sensor device 1410 may include an optical fiber having a fiber-end sensor 1411 disposed at or near a distal end thereof. The second fiber optic sensor device 1415 may be separate from the first fiber optic sensor device 1410 and may include an optical fiber having a plurality of fiber Bragg grating (FBG) sensors 1415a, 1415b, and 1415c disposed along a length thereof. Thus, the configuration of FIG. 14D may employ two separate optical fibers having different sensing functions. The fiber-end sensor 1411 is described above with reference to FIG. 14B, and the above description of the fiber-end sensor 1411 is incorporated herein. The plurality of fiber Bragg grating (FBG) sensors 1415a, 1415b, and 1415c may be as described above with reference to FIG. 14C, and the above description of the fiber Bragg grating (FBG) sensors 1413a, 1413b, and 1413c is incorporated herein.

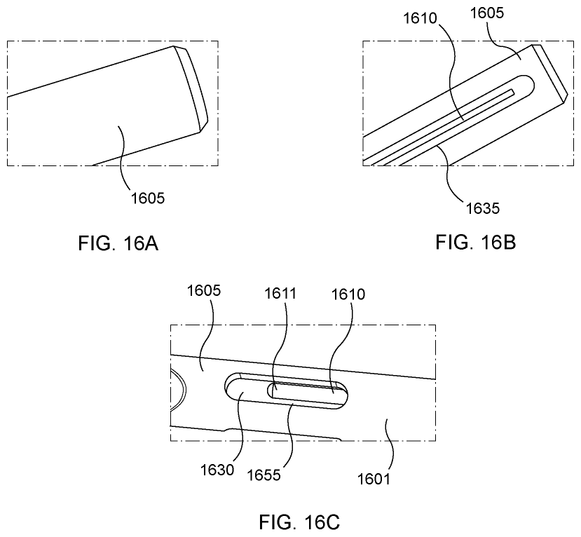

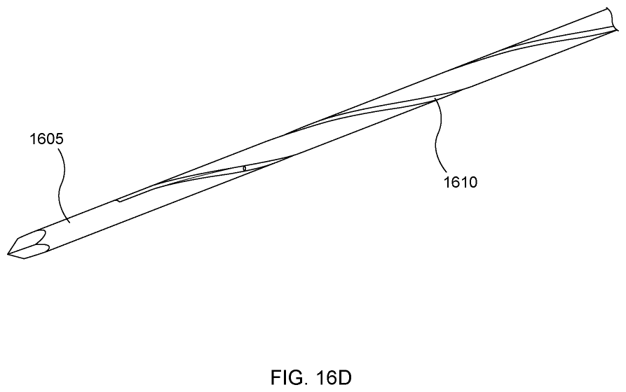

The first fiber optic sensor device 1410 and the second fiber optic sensor device 1415 may be arranged at different locations relative to the ablation probe 1420. For example, the fiber optic sensor devices 1410 and 1415 may be disposed on different portions of the ablation probe 1420, at different circumferential positions, at different longitudinal positions, or in other spatial arrangements selected to provide sensing in multiple regions surrounding the ablation probe 1420. In some embodiments, one or more of the fiber optic sensor devices 1410 and 1415 may be arranged in accordance with the configuration illustrated in FIG. 6C, for example, in which an optical fiber is disposed along and/or integrated with the ablation probe, including configurations in which the optical fiber is coupled to a surface, embedded within a structure, or otherwise secured relative to the ablation probe 1420. Further details for securing the fiber optic sensor devices to the ablation probe 1420 is provided below with reference to FIGS. 15A-16D.

In other embodiments, the number of fiber optic sensor devices disposed around the ablation probe 1420 may be varied depending on the coverage around the ablation probe 1420 that is desired and the circumference of the ablation probe 1420 and first fiber optic sensor. For example, two, three, four, or more fiber optic sensor devices may be distributed circumferentially around the ablation probe 1420 to provide partial or full circumferential coverage. In some embodiments, the angular spacing between adjacent fiber optic sensor devices may be uniform or non-uniform based on desired sensing resolution in particular regions. The number and arrangement of the fiber optic sensor devices may also be selected based on the diameter of the ablation probe 1420, available surface area, and routing constraints for the optical fibers. In some embodiments, increasing the number of fiber optic sensor devices may improve spatial resolution and more detailed temperature profiling, while fewer fiber optic sensor devices may be used to reduce device complexity, size, and cost. Additionally, the fiber optic sensor devices may be arranged in one or more circumferential rings and/or at multiple longitudinal positions along the ablation probe 1420 to provide three-dimensional sensing coverage.

In some embodiments, the FBG sensors 1415a-1415c of the second fiber optic sensor device 1415 may provide distributed measurements of temperature, strain, and/or pressure along the ablation probe 1420, while the fiber-end sensor 1411 of the first fiber optic sensor device 1410 may provide localized sensing at or near a distal treatment region. In some embodiments, the fiber-end sensor 1411 may be particularly suited for sensing acoustic signals and/or for imaging, including ultrasound-based imaging, acousto-optic imaging and/or optoacoustic imaging.

The first fiber optic sensor device 1410 and the second fiber optic sensor device 1415 may thereby provide complementary sensing. For example, the distributed measurements from the FBG sensors 1415a-1415c may be used to determine temperature gradients and estimate the location of a thermal exposure zone boundary 1430 and an ablation zone boundary 1440, while the fiber-end sensor 1411 may provide localized acoustic sensing and/or imaging at or near the distal end of the ablation probe 1420.

In some embodiments, the fiber-end sensor 1411 may be more susceptible to damage due to exposure to elevated temperatures and/or temperature gradients associated with operation of the ablation probe 1420. Accordingly, the position of the fiber-end sensor 1411 relative to the thermal exposure zone boundary 1430 and the ablation zone boundary 1440 may be managed based on temperature measurements obtained from the FBG sensors 1415a-1415c of the second fiber optic sensor device 1415. For example, the distributed temperature measurements may be used to ensure that the fiber-end sensor 1411 remains outside of a region exceeding a predetermined temperature threshold.

In some embodiments, feedback from the FBG sensors 1415a-1415c may be used to adjust operation of the ablation device 1400D and/or reposition the ablation probe 1420 to limit thermal exposure of the fiber-end sensor 1411. Additionally or alternatively, the sensing data may be used to dynamically determine safe operating conditions for the fiber-end sensor 1411 while maintaining effective ablation performance.

The first fiber optic sensor device 1410 and the second fiber optic sensor device 1415 may be optically coupled to one or more optical sensing systems, such as the configurations described with respect to FIGS. 11-13, to enable interrogation of the fiber-end sensor 1411 and the FBG sensors 1415a-1415c. In some embodiments, the two fiber optic sensor devices may be interrogated independently or by multiplexing techniques.

Accordingly, the configuration of FIG. 14D provides an ablation device in which a first fiber optic sensor device 1410 provides localized acoustic sensing and/or imaging and a second fiber optic sensor device 1415 provides distributed sensing, thereby enabling enhanced tracking, monitoring, guidance, temperature control and protection of sensing components during an ablation procedure.

FIGS. 15A-15C illustrate example configurations for placement and integration of one or more fiber optic sensor devices with an ablation probe 1420 of an ablation device, consistent with embodiments described herein. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

In some embodiments, the fiber optic sensor devices, such as the first fiber optic sensor device 1410 and the second fiber optic sensor device 1415, may be integrated with the ablation probe 1420 by being routed along the exterior surface, partially embedded within a channel formed in the probe, or disposed within an interior portion of the probe structure. The number of fiber optic sensor devices that may be incorporated with the ablation probe 1420 may depend on factors including the diameter of the ablation probe 1420, the outer diameter of the optical fibers, and spacing requirements between fibers, mechanical integrity, and functional requirements. In some examples, the ratio between fiber size and probe size determines how many fibers may be bundled or integrated with the probe.

In some embodiments, as shown in FIG. 15C, fiber optic sensor devices routed within an interior portion of the ablation probe 1420 may be limited to sensing configurations that do not require direct exposure to the surrounding environment. For example, fiber Bragg grating (FBG) sensor arrangements, such as FBG sensors 1417a-1417c and 1413a-1413c, may be disposed within a channel formed in the interior of the ablation probe 1420 for distributed temperature sensing. Placement of such fibers within the probe may be selected based on thermal characteristics of the device, including insulation or heat distribution along the probe, such that the fibers provide representative thermal measurements while avoiding excessive thermal exposure.

In contrast, sensing elements configured for acoustic signal sensing, including fiber-end sensors 1417d and 1413d, may be positioned such that they are exposed to acoustic signals generated during operation of the ablation probe 1420 and propagate from the ablation zone and/or the surrounding tissue region in response to energy delivery during ablation and imaging. For example, such sensors may be positioned at or near an exterior surface of the ablation probe 1420, including within channels, openings in the channels, or other regions that permit acoustic coupling between surrounding tissue and the sensing element. In some embodiments, acoustic sensing regions may be coupled with a potting material, such as a UV-curable potting material (e.g., Vitralit 6108) that is acoustically transmissive to facilitate detection of acoustic signals for tracking, positioning, and imaging applications, including OnPoint imaging. In contrast, FBG sensing arrangements sensing regions may not require such acoustically sensitive potting materials and may be encapsulated or protected using different materials optimized for thermal and mechanical stability.

In the embodiment illustrated in FIG. 15A, one or more fiber optic sensor devices 1410 and 1415 are embedded within one or more channels formed in an outer surface of the ablation probe 1420. The channels may extend longitudinally along the probe and may be dimensioned to receive the optical fibers, for example, within shallow grooves formed in the outer surface. The fibers may be routed along the ablation probe 1420 and along internal or external components used to control or deliver energy to a distal end of the ablation probe 1420. In some embodiments, the embedded configuration provides mechanical protection, reduces profile, maintains a smooth outer surface of the ablation probe 1420, thereby providing mechanical protection and maintaining a low-profile outer surface.

In the embodiment illustrated in FIG. 15B, one or more fiber optic sensor devices may be bundled with the ablation probe 1420 along an exterior surface thereof. In such configurations, the optical fibers may be secured to the probe using an adhesive or coating material, such as a dip coating including Loctite 4310, or other suitable bonding materials. The fibers may be arranged in parallel, helically wrapped, or otherwise distributed around the circumference of the ablation probe 1420 to achieve desired sensing coverage. In some embodiments, an outer coating may be applied over the fiber and probe assembly to provide mechanical stability, environmental protection, and improved biocompatibility.

FIG. 15C illustrates an additional configuration in which one or more fiber optic sensor devices 1410 and 1415 are disposed within one or more interior lumens of the ablation probe 1420. In some embodiments, optical fibers routed within the interior lumens may extend longitudinally through the probe and may be positioned alongside other components used for controlling and/or energizing the distal ablation portion of the instrument. Fiber optic sensor devices disposed within the interior lumens may include FBG sensing arrangements for distributed temperature sensing, strain sensing, and/or pressure sensing. In some embodiments, one or more windows may be formed in the wall of the lumen and/or the probe shaft to expose selected sensing regions of the optical fibers to the surrounding environment. For example, an acoustic sensing fiber or fiber-end sensor, such as 1417d and 1413d may be aligned with such a window so that acoustic signals from surrounding tissue may reach the sensing region, thereby enabling acoustic sensing, tracking, positioning, and/or imaging, while other portions of the fiber remain protected within the interior lumen. In some embodiments, the sensing region exposed through the window may be coupled with an acoustically transmissive potting material to facilitate acoustic coupling, while FBG sensing regions disposed within the interior lumen may remain unexposed and may be integrated without requiring such acoustically sensitive potting material.

In some embodiments, the optical fibers of the fiber optic sensor device 1410 may be secured to the ablation probe 1420 using a combination of mechanical retention features and bonding materials. For example, fibers disposed within channels may be further secured using adhesives, coatings, or potting materials, while fibers disposed externally may be adhered using dip coating processes, localized bonding, heat shrink tubing, or overmolding techniques. The selection of materials and attachment methods may depend on factors including mechanical robustness, acoustic transmission requirements, thermal tolerance, and manufacturing considerations.

In some embodiments, materials used for the fiber optic sensor devices, bonding agents, coatings, and potting materials may be selected to satisfy regulatory requirements for medical devices, including biocompatibility, sterilization compatibility, and shelf-life stability. For example, materials may be selected to comply with applicable FDA requirements and standards for implantable or insertable medical devices.

FIGS. 16A-16D illustrate example configurations of a fiber optic sensor device integrated with an ablation device, including structural features for acoustic coupling and protection of sensing elements, consistent with embodiments described herein. As shown in FIGS. 16A-16D, an ablation device 1605 may include a fiber optic sensor device 1610 having a fiber-end sensor 1611 disposed at or near a distal sensing region. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

In the embodiment illustrated in FIG. 16B, the fiber optic sensor device 1610 may be disposed along a surface of the ablation device 1605 and may be at least partially received within a channel 1635 formed in the device structure. The channel 1635 may extend longitudinally along the ablation device 1605 and may be configured to retain and protect the optical fiber while maintaining a low-profile integration with the device. In some embodiments, the channel 1635 may be dimensioned to closely match the diameter of the optical fiber, thereby improving alignment and mechanical stability.

As further illustrated in FIG. 16C, a lumen within the ablation device 1605 may include an opening or window 1655 that provides access between the interior of the device and the external environment. In some embodiments, the fiber optic sensor device 1610 may be routed through the lumen and positioned such that the fiber-end sensor 1611 is aligned with the window 1655. This configuration enables the fiber-end sensor 1611 to receive acoustic signals from surrounding tissue while the remainder of the optical fiber is protected within the lumen or channel.

In some embodiments, as illustrated in FIG. 16C, the fiber-end sensor 1611 and/or a sensing region of the fiber optic sensor device 1610 exposed through the window 1655 may be coupled with a potting material 1630.

The potting material 1630 may comprise an optically transparent and/or acoustically transmissive polymer configured to facilitate transmission of acoustic signals while providing mechanical support and environmental protection.

The potting material 1630 may be, for example, a UV-curable potting material such as Vitralit 6108, and may be selected to be acoustically transmissive to facilitate efficient transmission of acoustic signals to the fiber-end sensor 1611. In other examples, the potting compound may be one or more of Norland Optical Adhesive (e.g., NOA61), Norland-65 glue, Norland 81 glue, MY-132A polymer, MY-133, BIO-133, DC-133 or other suitable materials including acrylate-based, epoxy-based, or silicone-based polymers. The potting compound may be selected according to its acoustic and mechanical properties, for example, the speed of sound, acoustic impedance, thermal conductivity, water proofing, etc. In some embodiments, the potting material may be selected to have an acoustic impedance compatible with surrounding tissue or medium to enhance acoustic coupling, while also maintaining optical clarity for transmission and modulation of optical signals. The potting compound may also offer modification of acoustic impedance matching to the surrounding medium in addition to the mechanical fixing and protection of the sensor. The potting material 1630 may be cured to mechanically secure the sensing region and to provide acoustic coupling. The potting material 1630 may additionally provide mechanical stabilization and environmental protection for the sensing region while maintaining sensitivity to acoustic signals.

In some embodiments, the fiber optic sensor device 1610 is integrated with the ablation device 1605 without exposure of the sensing region through a window. In such configurations, the fiber optic sensor device 1610 may be fully enclosed within the device structure, for example within a lumen or along an interior or exterior surface, and may be configured for sensing modalities that do not require direct acoustic exposure, such as distributed temperature sensing using FBG sensors or other environmental parameter sensing.

FIG. 16D illustrates an embodiment in which the fiber optic sensor device 1610 is disposed along an exterior surface of the ablation device 1605 in a helical configuration. In some embodiments, one or more grooves may be formed in the outer surface of the ablation device 1605, and the optical fiber of the fiber optic sensor device 1610 may be received within the grooves and routed helically along a length of the ablation probe. The helical arrangement may provide distributed coverage around the circumference of the ablation probe while maintaining a low-profile integration with the device surface. In some embodiments, the optical fiber may be secured within the grooves using an adhesive, coating, or overmolding process, such as a dip coating material, to retain the fiber in place and protect it from mechanical damage. The helical routing may also increase the effective sensing length of the optical fiber within a given axial length of the ablation probe, thereby enhancing spatial resolution of distributed sensing, such as temperature sensing using FBG sensors.

In some embodiments, the configurations illustrated in FIGS. 16A-16D may be combined or modified to achieve desired sensing performance, mechanical robustness, and manufacturability. For example, multiple windows may be provided along the length of the ablation device 1605, multiple fiber optic sensor devices 1610 may be incorporated, and different sensing regions may be selectively exposed or protected depending on the sensing modality.

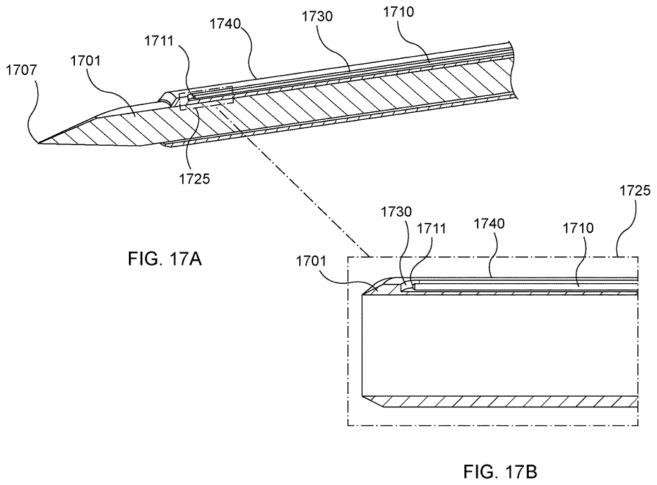

FIGS. 17A and 17B illustrates example configurations of an ablation device 1701 integrated with a fiber optic sensor device 1710, consistent with embodiments described herein. As shown in FIGS. 17A and 17B, the fiber optic sensor device 1710 includes a fiber-end sensor 1711 disposed at a distal region of the optical fiber and positioned proximal to an ablation tip 1707 of the ablation device 1701. FIG. 17A illustrates an example configurations of a fiber optic sensor device 1710 integrated with an ablation device 10. FIG. 17B illustrates an example of an enlarged view of region 1725 illustrating an examples of the fiber optic sensor device 1710 includes a fiber-end sensor 1711 positioned proximal to an ablation tip 1707 of the ablation device 1701. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

In some embodiments, the optical fiber of the fiber optic sensor device 1710 may be routed along the ablation device 1701, for example extending along a length of a hypotube associated with the ablation device. In some embodiments, the optical fiber may be disposed within a channel formed on an exterior surface of the hypotube. For example, the channel may have a depth of approximately 100 microns and a width of approximately 300 microns, sized to receive the optical fiber while maintaining a low-profile integration with the ablation device 1701.

In some embodiments, the dimensions of the channel may be varied based on the diameter of the optical fiber and the desired degree of recessing of the fiber within the hypotube. For example, the channel may have a depth in a range of approximately 50 microns to 200 microns and a width in a range of approximately 150 microns to 500 microns. In some embodiments, for an optical fiber having an outer diameter of approximately 80 microns to 150 microns, the channel depth may be approximately equal to or slightly less than the fiber diameter and the channel width may be approximately 1.5 to 3 times the fiber diameter. In other embodiments, the channel may be shallower to permit partial exposure of the optical fiber above the outer surface of the hypotube, or deeper to more fully recess and protect the optical fiber within the hypotube.

In some embodiments, the fiber-end sensor 1711 and/or a distal portion of the optical fiber may be bonded to a distal end region of the hypotube using a potting material 1730. The potting material 1730 may comprise a UV-curable material, such as Vitralit 6108, and may provide mechanical fixation and environmental protection for the sensing region.

In some embodiments, an outer coating 1740 may be applied along at least a portion of the ablation device 1701 and the fiber optic sensor device 1710. The outer coating 1740 may comprise, for example, a dip coating material such as Loctite 4310, and may extend along substantially the entire length of the hypotube to protect the optical fiber and the potted sensing region from mechanical damage and environmental exposure.

In some embodiments, a proximal portion of the optical fiber may be routed through a hub of the ablation device 1701 and coupled to an optical cable. For example, the hub may be connected with a strain relief to a jacketed optical cable having a length and diameter suitable for interfacing with an external system. In some embodiments, the optical cable may terminate in a connector configured to couple with an external sensing or imaging system.

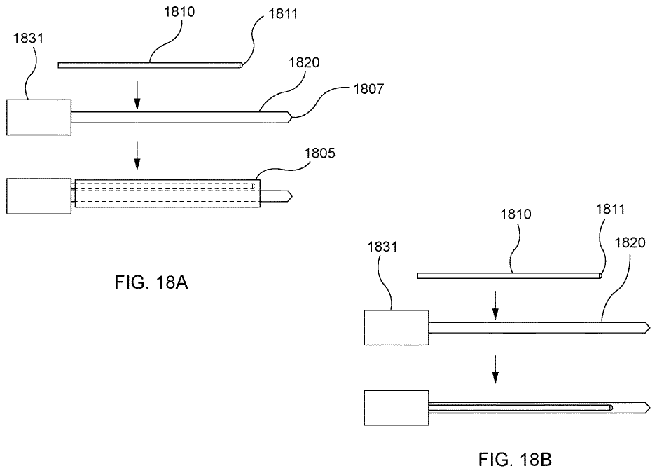

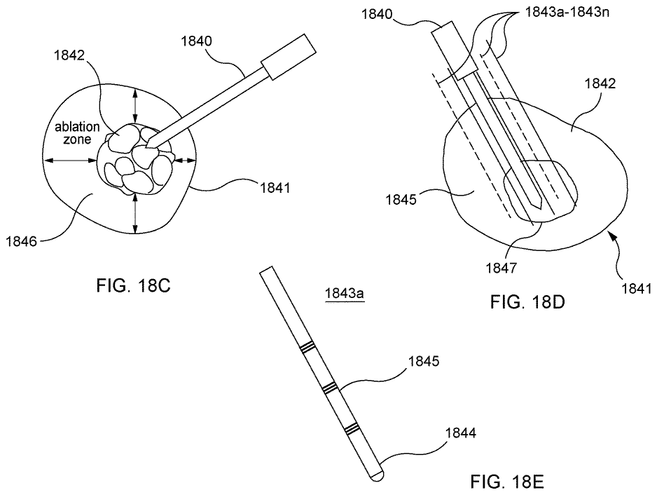

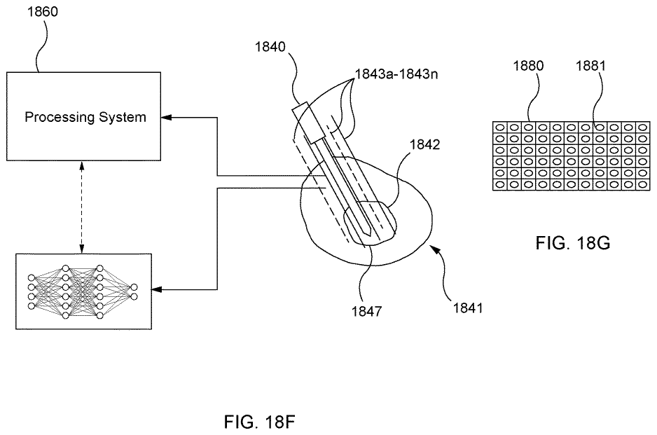

FIGS. 18A and 18B illustrate example configurations of a fiber optic sensor device integrated with an ablation device, consistent with embodiments described herein. As shown in FIGS. 18A and 18B, a fiber optic sensor device 1810 includes a fiber-end sensor 1811 disposed at a distal region thereof and positioned relative to an ablation probe 1820 extending from a body 1831 of an ablation device. The fiber-end sensor 1811 is disposed proximal to a tip 1807 of the ablation probe 1820. In some embodiments, the configurations, components, and operational details described with respect to the preceding figures are applicable to the embodiment of the present figure and are incorporated herein by reference. For the sake of brevity, such details are not repeated, and the present figure is described primarily with respect to its distinguishing features.

In the embodiment illustrated in FIG. 18A, the fiber optic sensor device 1810 is coupled to at least a portion of the ablation probe 1820, which extends from an ablation device body 1831. In some embodiments, the fiber optic sensor device 1810 may be disposed along an exterior surface of the ablation probe 1820 and positioned such that the fiber-end sensor 1811 is located at or near a distal portion of the ablation probe 1820 or proximal to the tip of the ablation probe 1820. The fiber optic sensor device 1810 may be secured to the ablation probe 1820 and/or the ablation device body 1831 using an attachment layer 1805. The attachment layer 1805 may comprise a polymer coating, adhesive, heat shrink tubing, lamination material, or combinations thereof, configured to secure the optical fiber while maintaining flexibility and mechanical integrity. In some embodiments, the attachment layer 1805 may extend along at least a portion of the fiber optic sensor device 1810 to protect the optical fiber and maintain its position relative to the ablation probe 1820. At least a portion of the fiber optic sensor device 1810 and the may be coated with a protective coating, such as a conformal coatings. Non-limiting examples of such conformal coatings which provide additional protection for the sensor portion and the optical fiber include polyamide, parylene, silicone, acrylic, polyethylene terephthalate (PET), fluorinated ethylene propylene (FEP), polytetrafluoroethylene (PTFE) and the like.

In some embodiments, the configuration of FIG. 18A may be similar to the external mounting arrangements described with respect to FIGS. 15A and 15B, in which the optical fiber is routed along an outer surface of the ablation probe 1820 and secured using coatings, adhesives, or other bonding techniques. Such configurations may enable exposure of the fiber-end sensor 1811 to the surrounding environment for sensing, including acoustic sensing, imaging, and/or environmental parameter measurement.

In the embodiment illustrated in FIG. 18B, the fiber optic sensor device 1810 is embedded into the ablation probe 1820 and/or the ablation device body 1831. In some embodiments, the ablation probe 1820 may include a recess, channel, or embedded region configured to receive the fiber optic sensor device 1810, such that the optical fiber is at least partially recessed within the structure of the ablation probe 1820. The fiber optic sensor device 1810 may be secured within the embedded region using the attachment layer 1805, which may comprise a polymer coating, adhesive, heat shrink tubing, lamination material, or combinations thereof.

In some embodiments, embedding the fiber optic sensor device 1810 within the ablation probe 1820 may provide improved mechanical protection, reduced profile, and enhanced durability of the optical fiber. The embedded configuration may also improve alignment and stability of the fiber-end sensor 1811 relative to the ablation probe 1820, while still enabling the fiber-end sensor 1811 to be positioned proximal to a tip of the ablation probe 1820.

In some embodiments, the configurations illustrated in FIGS. 18A and 18B may be selected based on desired sensing performance, manufacturability, and device robustness. For example, the externally mounted configuration of FIG. 18A may provide greater exposure of the fiber-end sensor 1811 for acoustic sensing and imaging, while the embedded configuration of FIG. 18B may provide increased protection and structural integration of the fiber optic sensor device 1810.

FIGS. 18C-16G illustrates an example configuration of a distributed fiber-optic sensing system deployed relative to an ablation probe for spatially resolved monitoring and verification of an ablation procedure.

As shown in FIGS. 18C-18D, an ablation probe 1840 may be positioned within a target tissue region 1841, defining an expected treatment volume 1842. A plurality of needle-based sensor delivery devices 1843a-1843n may be inserted into the tissue in a generally parallel or semi-parallel arrangement relative to the ablation probe 1840. In some embodiments, the needle-based sensor delivery devices 1843a-1843n may include needles or other elongated delivery members sized for minimally invasive placement within tissue. For example, the needle-based sensor delivery devices 1843a-1843n may have a gauge size of about 24 gauge or 25 gauge, or may have another size selected based on the anatomy, target location, sensing requirements, and/or ablation procedure. As illustrated in FIG. 18E, each needle-based sensor delivery device 1843a-1843n may include one or more fiber-optic sensors 1844 extending along at least a portion of its length, with one or more sensing regions 1845 positioned at known spatial locations.

The fiber-optic sensors 1844 may be arranged in a grid or other semi-regular pattern surrounding and/or extending through the expected treatment volume 1842, with at least some sensing regions 1845 positioned near a periphery 1846 of the expected treatment volume and others extending beyond a distal boundary 1847 of the ablation region. The sensing regions 1845 may be arranged to provide one or more sensing directions or measurement paths along which temperature, pressure, and/or tissue characterization data may be acquired over time and space.

In some embodiments, as illustrated in FIG. 18F, outputs from the fiber-optic sensors 1844 may be provided to a processing system 1860, which may include one or more computational models 1861, such as bioheat models based on the Pennes bioheat equation, finite element analysis models, and/or machine learning models such as those described with reference to FIGS. 19-20. The processing system 1860 may generate one or more outputs, including a thermal map, an ablation boundary, or a devascularized margin, which may be compared with a pre-treatment imaging boundary to assess efficacy and completeness of the treatment.

In some embodiments, as illustrated in FIG. 18G, the plurality of needle-based sensor delivery devices 1843a-1843n may be supported by a structured deployment device 1880, such as a grid or template having regularly spaced apertures 1881, to facilitate controlled placement of the sensors within the tissue.

In some embodiments, the apertures 1881 may be irregularly spaced. For example, the spacing between adjacent apertures 1881 may vary to accommodate anatomical constraints, target geometry, anticipated treatment margins, tissue heterogeneity, physician preference, or a desired sensing density in one or more regions of interest. In this manner, the structured deployment device 1880 may support placement patterns that are uniform in some regions and non-uniform in other regions, thereby permitting more tailored positioning of the needle-based sensor delivery devices 1843a-1843n relative to an expected ablation zone, a tumor margin, a devascularized margin, or other treatment boundary.

FIG. 19 illustrates an example processing architecture 1900 for integrating sensing, prediction, imaging, and control operations of the ablation system, consistent with embodiments described herein. As shown in FIG. 19, the processing architecture 1900 may receive multimodal inputs including FBG sensor inputs 1910, fiber-end sensor inputs 1920, prior data/content 1940, and device parameters 1930. These inputs may be processed by a feature extraction and fusion module 1960 and provided to a machine learning model 1950 configured to generate outputs including thermal and acoustic prediction through the thermal and acoustic prediction module 1970, zone boundary determination through the zone boundary determination module 1980, and control outputs through the control outputs module 1990. In some embodiments, the processing architecture 1900 operates as a closed-loop system in which the control outputs are used to adjust operation of the ablation device and sensing components, with updated sensing data continuously fed back into the system for real-time monitoring, prediction, and control.

In some embodiments, machine learning models may be trained to predict thermal gradients, acoustic signatures, and/or tissue responses, and to generate control signals for adjusting energy delivery, probe positioning, and/or sensor operation in real time for the ablation device, such as ablation device 1400D. In some embodiments, the machine learning models may be implemented as part of a processing architecture 1900 (FIG. 19) that receives sensed data from the FBG sensors 1415a-1415c and the fiber-end sensor 1411 as model inputs and generates predicted procedural states and/or control variables as model outputs. For example, the model inputs may correspond to FBG sensor inputs 1910 and fiber-end sensor inputs 1920, and may include wavelength shifts, reflected intensity changes, phase information, temperature values derived from the FBG sensors 1415a-1415c, acoustic response data derived from the fiber-end sensor 1411, and optionally time-series, frequency-domain, or time-frequency features generated therefrom. Additional inputs may include prior data and/or content 1940 and device parameters 1930, such as power level, duty cycle, pulse duration, frequency, probe position, elapsed treatment time, and/or previously estimated locations of the thermal exposure zone boundary 1430 and the ablation zone boundary 1440. Based on such inputs, the machine learning models may generate outputs corresponding to thermal and acoustic prediction through the thermal and acoustic prediction module 1970, zone boundary determination through the zone boundary determination module 1980, and control outputs through the control outputs module 1990, including predicted thermal gradients, predicted future temperature distributions, classifications of acoustic signatures, acoustic imaging, estimates of tissue state, estimated proximity of the fiber-end sensor 1411 to the thermal exposure zone boundary 1430 or the ablation zone boundary 1440, and recommended or automatically generated control signals for adjusting energy delivery, probe positioning, probe guidance and/or sensor operation.

In some embodiments, the processing architecture 1900 may include an input stage 1910, 1920, 1930, and 1940, a feature extraction and fusion stage, a machine learning inference stage implemented by the machine learning model 1950, and an output stage. For example, raw signals from the FBG sensors 1415a-1415c and the fiber-end sensor 1411 may first be preprocessed to extract features representative of slower thermal effects and faster acoustic effects, after which the features may be fused into a common representation by the feature extraction and fusion module 1960 for input to the machine learning model 1950. The inference stage may then generate one or more outputs, including imaging, monitoring, prediction, classification, and/or closed-loop control outputs. In this manner, the machine learning models may be integrated into a robust sensing and control architecture of any of FIGS. 11-13, as further illustrated in FIG. 19, which enables independent extraction of dynamic acoustic responses and slower thermal effects while also supporting prediction of boundary locations, protection of the fiber-end sensor 1411, and control of the ablation procedure.

In some embodiments, the machine learning models provide a technical improvement in the robust sensing and control architecture of any of FIGS. 11-13 by enabling real-time fusion of distributed optical sensing data (e.g., FBG sensor inputs 1910) and localized acoustic sensing data (e.g., fiber-end sensor inputs 1920) within the processing architecture 1900 to generate more accurate and responsive control of energy delivery and guidance. For example, the machine learning models improve the ability of the system to distinguish between thermal effects and acoustic phenomena, thereby enabling more accurate image creation, more precise estimation of ablation zone boundaries (e.g., via zone boundary determination module 1980) and reducing uncertainty in sensor measurements.

In some embodiments, the machine learning models improve the functioning of the ablation device itself by enabling dynamic adjustment of operating parameters based on predicted thermal gradients and tissue responses, such as those generated by the thermal and acoustic prediction module 1970, thereby reducing the risk of unintended thermal damage to surrounding tissue and to sensing components, including the fiber-end sensor 1411. Such improvements may result in enhanced safety, increased procedural accuracy, and improved reliability of the sensing system.

In some embodiments, the machine learning models provide improved signal processing capabilities that are not achievable using conventional threshold-based or rule-based approaches. For example, the models, in conjunction with the feature extraction and fusion module 1950, may extract and separate overlapping acoustic and thermal signals from optical sensing data, thereby improving signal fidelity and enabling more accurate interpretation of sensing data under complex and dynamic procedural conditions.

In some embodiments, the integration of the machine learning models with the fiber optic sensing architecture enables a closed-loop control system that continuously adapts to changing procedural conditions, as illustrated by the closed-loop feedback and control in FIG. 19, thereby improving control responsiveness and stability of the ablation process

In other embodiments, the machine learning models may receive time-series data from the FBG sensors 1415a-1415c and the fiber-end sensor 1411 and may output predictions of current and future temperature distributions, rates of temperature change, and/or likelihood of exceeding a predetermined thermal threshold, as part of the thermal and acoustic prediction outputs of the thermal and acoustic prediction module 1970. In some embodiments, the machine learning models may also classify acoustic signatures associated with cavitation events, tissue disruption, or other procedural states, and correlate such signatures with corresponding thermal conditions, acoustic information, and tissue responses.

In some embodiments, the machine learning models may be configured to generate control outputs through the control outputs module 1990 that adjust one or more operating parameters of the ablation device 1400D, including power level, duty cycle, pulse duration, frequency, or energy delivery profile, to maintain desired ablation performance while limiting thermal exposure to the fiber-end sensor 1411 and surrounding tissue. Additionally or alternatively, the control outputs may be used to guide repositioning of the ablation probe 1420, including advancing, retracting, or reorienting the probe based on predicted spatial distributions of temperature and/or acoustic activity.

In some embodiments, the machine learning models may operate in a closed-loop control system, in which sensed data is continuously used to update predictions and refine control actions in real time. For example, discrepancies between predicted and measured temperature or acoustic signals may be used to update model parameters and improve prediction accuracy during the procedure. In some embodiments, the models may incorporate safety constraints, such as maintaining the fiber-end sensor 1411 outside of the thermal exposure zone boundary 1430 or limiting exposure to temperatures above a predetermined threshold, such as 60° C. while optimizing ablation efficiency. In other examples, the predetermined threshold temperature may be 65° C., 50° C., 55° C., and 40° C., for example.

In some embodiments, the machine learning models may further integrate prior data and/or content 1940, including historical data from prior procedures, patient-specific information, and/or device-specific characteristics, to improve prediction accuracy and control performance. For example, the models may adapt to variations in tissue properties, probe geometry, or energy delivery modalities, thereby enabling more robust and personalized control of the ablation procedure.

In some embodiments, the machine learning models may be further configured to generate one or more images based on acoustic signals detected by the fiber-end sensor 1411 (e.g., fiber-end sensor inputs 1920) in combination with environmental parameters obtained from the FBG sensors 1415a-1415c. For example, acoustic response data captured at the fiber-end sensor 1411 may be processed to reconstruct spatial representations of tissue structures, while temperature and/or pressure information obtained from the FBG sensors 1415a-1415c may be incorporated to provide contextual information regarding tissue state, thermal gradients, and/or ablation boundaries. In this manner, the generated images may reflect both structural and environmental characteristics of the tissue.

In some embodiments, image generation may be performed by the machine learning model 1950 or alternatively by a separate model within the processing architecture 1900 configured specifically for image reconstruction. For example, a first model may perform signal separation and feature extraction to distinguish acoustic and thermal components, while a second model may utilize the extracted acoustic features, optionally in combination with FBG-derived environmental data, to generate two-dimensional or three-dimensional images of the treatment region. In some embodiments, such models may include convolutional neural networks, or other models configured for acoustic image reconstruction, thereby enabling enhanced visualization, guidance, and monitoring of the ablation procedure.

The generation of images based on acoustic signals from the fiber-end sensor 1411, optionally in combination with environmental parameters from the FBG sensors 1415a-1415c, as implemented within the processing architecture 1900 of FIG. 19, provides a concrete technological improvement in the field of ablation systems by enabling real-time visualization of tissue structure, ablation boundaries, and probe position during a procedure. Such image generation is not merely an abstract data analysis, but instead transforms sensor-derived physical signals into clinically actionable representations that improve guidance, accuracy, and safety of the ablation probe 1420. By integrating multimodal sensing and image reconstruction within a sensor-hostile ablation environment, the disclosed system enhances device functionality and procedural outcomes, thereby providing a practical application that addresses a technical problem in medical device operation.

FIG. 20 illustrates an example hardware architecture 2000 for implementing the machine learning model and processing operations described with respect to FIG. 19. The hardware architecture 2000 may be configured to receive multimodal sensing inputs, perform feature extraction and machine learning inference, and generate control outputs for operation of the ablation system.

In some embodiments, the hardware architecture 2000 may include a system bus 2005, one or more processors 2090, memory 2080, a database 2085, and one or more hardware accelerators 2070 coupled to the system bus 2005. The processors 2090 may include one or more central processing units (CPUs), microcontrollers, or digital signal processors (DSPs) configured to execute instructions for signal processing, feature extraction, and control operations. The memory 2080 may include volatile and/or non-volatile memory configured to store program instructions, model parameters, calibration data, and/or historical sensing data. In some embodiments, the database 2085 may store patient-specific information, historical procedural data, device configuration data, training data, and/or other data used by the machine learning model and control system.

In some embodiments, the hardware accelerators 2070 may include one or more graphics processing units (GPUs), tensor processing units (TPUs), field-programmable gate arrays (FPGAs), or application-specific integrated circuits (ASICs) configured to accelerate execution of machine learning models. The hardware accelerators 2070 may be configured to execute inference operations of the machine learning model (e.g., model 1950) and, in some embodiments, may also support on-device training or model updates. In some embodiments, the processors 2090, memory 2080, database 2085, and hardware accelerators 2070 may communicate over the system bus 2005 to support coordinated processing, storage, retrieval, and control operations within the hardware architecture 2000.

In some embodiments, the hardware architecture 2000 may further include a sensing interface 2010 configured to receive sensor data from the fiber optic sensing system, including FBG sensor inputs (e.g., 1910) and fiber-end sensor inputs (e.g., 1920). The sensing interface 2040 may include optical receivers, analog-to-digital converters, and/or signal conditioning circuitry configured to convert optical and/or electrical signals into digital data suitable for processing.

In some embodiments, the hardware architecture 2000 may further include an operational and contextual data module 2020 configured to receive and manage multiple sources of input data for use by the sensing and control system. For example, the operational and contextual data module 2020 may receive parameters of the ablation device (e.g., 1930), including power level, pulse duration, duty cycle, probe position, and other operating conditions, as well as prior data and content (e.g., 1940), including patient-specific information, historical procedural data, and previously acquired sensing data. In some embodiments, the operational and contextual data module 2020 may further receive feedback-driven control information generated by the machine learning model and/or control outputs, including adjustments to energy delivery, probe positioning, and sensor operation. The operational and contextual data module 2020 may be configured to aggregate, normalize, and provide such data to downstream processing components, including feature extraction and fusion module 2030 and machine learning models 2040, thereby enabling context-aware prediction, adaptive control, and improved accuracy of sensing and imaging operations within the ablation system.

In some embodiments, the hardware architecture 2000 may include a feature processing module 2030 configured to perform preprocessing and feature extraction on the received sensing data. The feature processing module 2030 may be implemented in software executed by the processors 2090 and/or in dedicated hardware, and may generate time-domain, frequency-domain, and/or time-frequency features for input to the machine learning model. In some embodiments, preprocessing operations may include signal conditioning, noise filtering, normalization, synchronization of signals from multiple sensors, and segmentation of time-series data. The feature extraction operations may derive features representative of both relatively slower thermal responses and relatively faster acoustic responses, including amplitude, phase, spectral components, and temporal patterns. In some embodiments, the feature processing module 2030 may further perform feature selection, dimensionality reduction, and/or fusion of features obtained from different sensing modalities to generate a unified representation for input to the machine learning model.

In some embodiments, the hardware architecture 2000 may include a machine learning inference engine 2040 configured to execute the machine learning model (e.g., ML model 1950) using the processed features. The inference engine 2040 may be implemented using the processors 2090 and/or the hardware accelerators 2070 and may generate outputs corresponding to thermal and acoustic prediction, zone boundary determination, and control outputs. In some embodiments, the inference engine 2040 may also generate image outputs based on acoustic signals detected by the fiber-end sensor, optionally in combination with environmental parameter data obtained from one or more FBG sensors. Such image outputs may include two-dimensional or three-dimensional acoustic images representing tissue structure, ablation boundaries, and/or probe position.

In some embodiments, the hardware architecture 2000 may include a control interface 2050 configured to generate and transmit control signals to the ablation device, including signals for adjusting energy delivery, probe positioning, and/or sensor operation. The control interface 2050 may include digital-to-analog converters, communication interfaces, and/or control circuitry configured to interface with the ablation probe and associated systems.

In some embodiments, the hardware architecture 2000 may further include a communication interface 2060 configured to communicate with external systems, including user interfaces, remote computing systems, data storage systems, or other machine learning or image processing systems. The communication interface 2060 may support wired and/or wireless communication protocols.

In some embodiments, the components of the hardware architecture 2000 may be integrated within the ablation device, provided as part of an external console connected to the ablation device, or distributed across multiple systems. For example, real-time inference may be performed locally within the ablation system, while training or model updates may be performed on a remote system.

Accordingly, the hardware architecture 2000 provides a physical implementation of the machine learning processing described with respect to FIG. 19, enabling real-time processing of multimodal sensing data and generation of improved control outputs for guiding operation of the ablation system.

In another embodiment, interventional instrumentation, e.g., a minimally invasive tool, integrates two distinct optical fiber-based sensors—one configured for ultrasound detection and the other for temperature measurement—enabling simultaneous and independent monitoring of acoustic waves and thermal changes. The ultrasound sensor operates in the time domain by detecting oscillations in the intensity of optical signals corresponding to high-frequency acoustic waves, while the temperature sensor operates in the frequency domain by monitoring the wavelength or frequency shift of optical signals induced by thermal variations. This dual-sensor configuration allows decoupling of the acoustic and thermal signals based on their distinct temporal and spectral characteristics. The temperature data derived from the frequency shift can be used to calibrate and compensate for thermal effects on the acoustic sensor's signal, significantly improving the accuracy and reliability of ultrasound detection in environments with fluctuating temperatures. This architecture is particularly advantageous for applications such as thermal ablation, where precise acoustic monitoring must be maintained alongside real-time temperature feedback. The described method represents one implementation of simultaneous multimodal sensing using fiber-optic sensors and does not preclude other configurations or signal processing techniques capable of achieving similar outcomes.

The systems and devices provided herein may provide significant advantages for use during ablation procedures. Each of the various procedures, methods, and functionalities discussed below may provide advantages during ablation procedures and may employ various combinations of fiber optic sensor devices as described herein. The fiber optic sensor devices described herein may be combined in various ways to permit any and all of the various procedures, methods, and functionalities to be combined for performance by a single device. Some such devices may include fiber optic sensor devices operating in multimodal fashion. This type of operation may simplify device design, as fewer devices are required. Due to the above-discussed compact size of the fiber optic sensor devices discussed herein, it may also be convenient to combine multiple fiber optic devices, each optimized for measurement of one or more of the parameters discussed herein.

In embodiments, the systems and devices herein may provide tracking, location, and/or guidance. Some techniques may be discussed in U.S. Pat. No. 12,025,489, titled FIBER-OPTICAL SENSOR SYSTEM FOR ULTRASOUND SENSING AND IMAGING, issued on Jul. 2, 2024, U.S. patent application Ser. No. 18/382,984, titled TRANSPONDER TRACKING AND ULTRASOUND IMAGE ENHANCEMENT, filed on Oct. 23, 2023 and published as U.S. Patent Pub. No. 2024/0423482, and U.S. patent application Ser. No. 18/698,193, titled ULTRASOUND BEACON VISUALIZATION WITH OPTICAL SENSORS, filed on Oct. 7, 2022 and published as U.S. Patent Pub. No. 2025/0235180, each of which is incorporated herein by reference. Tracking, location, and guidance may be provided through ultrasound techniques discussed herein. Fiber optic sensor devices with acoustic sensing capabilities may be used to provide real-time location of objects on which they are located. Thus, when coupled with ablation devices, fiber optic sensor devices may be employed to guide ablation devices to ablation targets. Additionally, as discussed above, fiber optic sensor devices may be employed to provide ultrasound visualization from the point of view of an ablation device, thus increasing the ability to guide an ablation procedure. Further, in embodiments, an acoustic reflection of an ablation device, e.g., as measured by external ultrasound and/or by fiber optic sensor devices located within the tissue, may be used for location, guidance, and tracking.

Ablation zone mapping may be facilitated by various applications of the present technology. As discussed above and throughout, distributed sensors along fiber optic sensor devices may be employed to detect or measure temperature at multiple points within the tissue. This information may be used to map temperature gradients and isothermal zones, which may be indicative of ablation procedure progress. In further embodiments, isothermal zones may be measured through acoustic signals, e.g., by measurement of time of arrival changes that may be indicative of speed of sound changes in tissue caused by ablation procedures. In embodiments, temperature measurements made by fiber optic sensor devices may be combined with acoustic signal measurements indicative of temperatures to provide further detail and confidence in ablation zone mapping. In some embodiments, one or more fiber optic sensor devices may be anchored in tissue during a procedure to provide ablation zone monitoring and tracking throughout the length of a procedure.

Tissue pressure measurements provided by fiber optic sensors devices disclosed herein may also provide information related to ablation progress. During ablation procedures, as tissue is ablated, tissue pressure may change. As described herein, fiber optic sensor devices may be used to detect pressure and pressure changes, e.g., elastography. This information may be used to measure/monitor an ablation procedure. In embodiments that include distributed optical sensors, measurement of pressure changes at multiple locations may be used for pressure mapping. In embodiments, these tissue pressure measurements may be combined with tissue pressure measurements made via acoustic signal detection, including techniques such as strain wave or shear wave elastography and acoustic radiation force impulse imaging.

In embodiments, information captured through acoustic signal detection provided by internally located fiber optic sensor devices may be used with doppler techniques for blood flow measurement. Doppler ultrasound may be used to measure localized blood flow. Such techniques may be performed by fiber optic sensor devices, as discussed herein, to measure blood flow in an ablation region. Blood flow may be an important factor in selecting an appropriate ablation site as well as in monitoring ablation progress.

In embodiments, information captured through acoustic signal detection provided by internally located fiber optic sensor devices may be used to track or identify bubbles created through thermal ablation. Such techniques may further employ contrast enhanced ultrasound methods. Bubble tracking/identification may be used to monitor ablation progress.

In embodiments, information captured through acoustic signal detection provided by internally located fiber optic sensor devices may be combined according to sensor fusion methods. Such methods may permit the production of improved ultrasound images through the use of information captured by multiple different fiber optic sensor devices provided at different locations. Further, such information may be combined with information captured via external ultrasound devices for further enhancement. Additionally, non-imaging ultrasound techniques (e.g., doppler techniques, elastography techniques, etc.) may be employed by one or more fiber optic sensor devices and/or by one or more externally located devices. Non-imaging ultrasound information captured from any of these devices may be combined via sensor fusion techniques.

Embodiment 1 is a method of manufacturing an ablation device, comprising forming an ablation probe having a proximal portion and a distal portion comprising a distal tip, forming a channel in an outer surface of the ablation probe, disposing at least one optical fiber of a fiber optic sensor device within the channel, positioning a sensing region of the optical fiber proximal to the distal tip of the ablation probe, and securing the optical fiber to the ablation probe.

Embodiment 2 is the method of embodiment 1, wherein securing the optical fiber comprises applying an adhesive coating to the ablation probe and/or the optical fiber.

Embodiment 3 is the method of embodiment 2, wherein the adhesive coating comprises a dip coating applied along at least a portion of the ablation probe.

Embodiment 4 is the method of embodiment 1, further comprising disposing a plurality of optical fibers around a circumference of the ablation probe based on a ratio between a diameter of the ablation probe and a diameter of the optical fibers.

Embodiment 5 is the method of embodiment 1, wherein the optical fiber comprises at least one fiber Bragg grating (FBG) sensor disposed along a length of the optical fiber.

Embodiment 6 is the method of embodiment 1, wherein the sensing region comprises a fiber-end sensor configured to detect acoustic signals.

Embodiment 7 is the method of embodiment 6, further comprising coupling the fiber-end sensor to an acoustically transmissive material.

Embodiment 8 is the method of embodiment 1, further comprising forming the channel as a recessed groove sized to receive the optical fiber and applying a bonding agent to retain the optical fiber within the channel.

Embodiment 9 is the method of embodiment 1, wherein disposing the optical fiber comprises arranging the optical fiber in a longitudinal, circumferential, or helical configuration relative to the ablation probe.

Embodiment 10. A method of manufacturing an ablation device, comprising forming an ablation probe having a proximal portion and a distal portion comprising a distal tip, forming a lumen within the ablation probe, disposing at least one optical fiber of a fiber optic sensor device along the ablation probe, including at least partially within the lumen, positioning a sensing region of the optical fiber proximal to the distal tip of the ablation probe, and securing the optical fiber to the ablation probe.

Embodiment 11 is the method of embodiment 10, further comprising forming an opening or window in a wall of the lumen, and aligning the sensing region of the optical fiber with the window such that the sensing region is exposed to an external environment.

Embodiment 12 is the method of embodiment 11, further comprising disposing a potting material within the window to couple the sensing region of the optical fiber to the external environment.

Embodiment 13 is the method of embodiment 12, wherein the potting material comprises a UV-curable material configured to be acoustically transmissive.

Embodiment 14 is the method of embodiment 13, further comprising curing the potting material to mechanically secure the sensing region and provide acoustic coupling.

Embodiment 15 is the method of embodiment 10, wherein disposing the optical fiber within the lumen comprises routing the optical fiber alongside one or more components configured to deliver energy to the distal portion of the ablation probe.

Embodiment 16 is an ablation system, comprising an ablation probe, a sensing system associated with the ablation probe and comprising at least one temperature sensor configured to generate temperature-related data, and at least one acoustic sensor configured to generate acoustic data, and one or more processors configured to determine a temperature profile based on the temperature-related data, and control at least one of operation, sensing, or positioning of the acoustic sensor based on the temperature profile.

Embodiment 17 is the system of embodiment 16, wherein the sensing system comprises an optical fiber, and wherein the at least one temperature sensor comprises a plurality of fiber Bragg grating (FBG) sensors disposed along the optical fiber and configured to generate the temperature-related data, and the at least one acoustic sensor comprises a fiber-end sensor disposed at a distal portion of the optical fiber and configured to generate the acoustic data.

Embodiment 18 is the system of embodiment 17, wherein the one or more processors are further configured to control positioning of the fiber-end sensor relative to a thermal exposure zone based on the temperature profile.

Embodiment 19 is the system of embodiment 17, wherein the one or more processors are further configured to maintain the fiber-end sensor outside of a region exceeding a predetermined temperature threshold.

Embodiment 20 is the system of embodiment 19, wherein the predetermined temperature threshold corresponds to a thermal exposure zone boundary.

Embodiment 21 is the system of embodiment 17, wherein the one or more processors are further configured to determine a distance between the fiber-end sensor and a thermal exposure zone boundary based on the temperature profile.

Embodiment 22 is the system of embodiment 17, wherein the one or more processors are further configured to adjust a position of the ablation probe to limit thermal exposure of the fiber-end sensor.

Embodiment 23 is the system of embodiment 17, further comprising a wavelength division multiplexing (WDM) module configured to simultaneously interrogate the plurality of FBG sensors and the fiber-end sensor.

Embodiment 24 is the system of embodiment 17, wherein each of the plurality of FBG sensors is each associated with a respective wavelength channel.

Embodiment 25 is the system of embodiment 17, wherein acoustic signal modulation at the fiber-end sensor is encoded in at least one wavelength channel.

Embodiment 26 is the system of embodiment 25, further comprising a plurality of photodetectors configured to detect optical signals associated with the respective wavelength channels.

Embodiment 27 is the system of embodiment 17, further comprising a switch configured to selectively couple the optical fiber to one or more sensors.

Embodiment 28 is the system of embodiment 27, wherein the switch comprises a micro-electro-mechanical system (MEMS) switch.

Embodiment 29 is the system of embodiment 28, wherein one or more processors are further configured to control the switch to selectively interrogate the plurality of FBG sensors and the fiber-end sensor.

Embodiment 30 is the system of embodiment 17, wherein the one or more processors are further configured to correlate acoustic data from the fiber-end sensor with the temperature profile to determine a tissue state.

Embodiment 31 is the system of embodiment 16, wherein the one or more processors are further configured to generate control signals for adjusting energy delivery based on both the temperature profile and acoustic data.

Embodiment 32 is the system of embodiment 17, wherein the one or more processors are further configured to determine a thermal gradient and adjust positioning of the fiber-end sensor based on the thermal gradient.

Embodiment 33 is a method of operating an ablation system, comprising obtaining temperature-related data from a plurality of FBG sensors disposed along an optical fiber, determining a temperature profile along an ablation probe based on the temperature-related data, obtaining acoustic data from a fiber-end sensor disposed at a distal portion of the optical fiber, and controlling at least one of positioning or operation of the fiber-end sensor based on the temperature profile.

Embodiment 34 is an ablation system, comprising an ablation probe, an optical fiber extending along at least a portion of the ablation probe, a plurality of fiber Bragg grating (FBG) sensors disposed along the optical fiber and configured to generate temperature-related data, a fiber-end sensor disposed at a distal portion of the optical fiber and configured to sense acoustic signals, and one or more processors configured to determine a temperature profile along the ablation probe based on the temperature-related data from the plurality of FBG sensors, and control at least one of positioning or operation of the fiber-end sensor based on the temperature profile.

Embodiment 35 is an ablation system, comprising an ablation probe, a fiber optic sensing system comprising at least one fiber Bragg grating (FBG) sensor disposed on the ablation probe, the FBG sensor being configured to generate FBG sensor inputs including temperature-related data, and a fiber-end sensor disposed on a distal portion of the ablation probe, the FBG sensor being configured to generate fiber-end sensor inputs including acoustic data, and one or more processors configured to receive the FBG sensor inputs and the fiber-end sensor inputs, process the FBG sensor inputs and the fiber-end sensor inputs using a machine learning model, and generate, based on the processing, one or more outputs including at least one of a thermal prediction, an acoustic output, a zone boundary determination, or a control output for the ablation probe.

Embodiment 36 is the ablation system of embodiment 35, wherein the one or more processors is further configured to receive prior data and content including patient-specific data, historical data, or procedural data.

Embodiment 37 is the ablation system of embodiment 35, wherein the one or more processors is configured to receive device parameters including one or more of power level, pulse duration, duty cycle, or probe position.

Embodiment 38 is the ablation system of embodiment 35, wherein the one or more processors comprises a feature extraction and fusion module configured to generate fused representations of the FBG sensor inputs and the fiber-end sensor inputs for the machine learning model.

Embodiment 39 is the ablation system of embodiment 38, wherein the feature extraction and fusion module is further configured to separate thermal signals from acoustic signals based on differences in temporal, wavelength, or frequency characteristics.

Embodiment 40 is the ablation system of embodiment 35, wherein the machine learning model is further configured to generate thermal predictions including at least one of a thermal profile, a temperature distribution, or a rate of temperature change.

Embodiment 41 is the ablation system of embodiment 35, wherein the machine learning model is further configured to generate acoustic outputs including acoustic imaging.

Embodiment 42 is the ablation system of embodiment 35, wherein the machine learning model is further configured to determine a zone boundary including at least one of a thermal exposure zone boundary or an ablation zone boundary.

Embodiment 43 is the ablation system of embodiment 35, wherein the control output comprises at least one of an energy adjustment signal, probe positioning instruction, or guidance signal.

Embodiment 44 is the ablation system of embodiment 35, wherein the control output is used to adjust operation of the ablation probe and to updated sensor inputs that are fed back into the machine learning model.

Embodiment 45 is the ablation system of embodiment 35, wherein the machine learning model is configured to generate an image based on the fiber-end sensor inputs.

Embodiment 46 is the ablation system of embodiment 45, wherein the image comprises an acoustic image representing tissue structure and/or ablation boundaries.

Embodiment 47 is the ablation system of embodiment 35, wherein the machine learning model is configured to estimate a proximity of the fiber-end sensor to a thermal exposure zone boundary.

Embodiment 48 is the ablation system of embodiment 35, wherein the machine learning model is configured to limit exposure of the fiber-end sensor to temperatures above a predetermined threshold.

Embodiment 49 is the ablation system of embodiment 35, wherein the machine learning model comprises a neural network configured to process multimodal sensing data.

Embodiment 50 is a method of operating an ablation system, comprising receiving FBG sensor inputs including temperature-related data from at least one fiber Bragg grating sensor, receiving fiber-end sensor inputs including acoustic data from a fiber-end sensor, processing the FBG sensor inputs and the fiber-end sensor inputs using a machine learning model, and generating, based on the processing, one or more outputs including at least one of a thermal prediction, an acoustic output, a zone boundary determination, or a control signal for controlling an ablation probe.

Embodiment 51 is the method of embodiment 50, further comprising generating fused features from the FBG sensor inputs and the fiber-end sensor inputs prior to processing by the machine learning model.

Embodiment 52 is the method of embodiment 51, further comprising adjusting one or more operating parameters of the ablation probe based on the control signal.

Embodiment 53 is an ablation system, comprising an ablation device configured to deliver energy to a target tissue region, one or more sensor devices configured to be positioned within or adjacent to the target tissue region, and a processing system configured to receive sensor inputs from the one or more sensor devices and to generate, based on the sensor inputs, one or more outputs associated with an ablation procedure, wherein the sensor inputs include at least one of temperature data, pressure data, or tissue characterization data, and wherein the one or more sensor devices are arranged to provide spatially distributed measurements within or adjacent to an estimated treatment volume of the ablation device.

Embodiment 54 is the system of embodiment 53, wherein the one or more sensor devices are supported by a positioning structure configured to control placement of the sensor devices within the target tissue region, the positioning structure comprising a plurality of openings arranged in a regular, semi-regular, or irregular pattern.

Embodiment 55 is the system of embodiment 53, wherein the processing system comprises one or more models configured to process the sensor inputs and to generate the one or more outputs, the one or more outputs comprising at least one of a thermal distribution, an ablation boundary, a tissue characterization map, or a treatment verification output.

Embodiment 56 is the system of embodiment 53, wherein each of the one or more sensor devices comprises an optical sensor having a plurality of sensing regions distributed along a portion of the sensor device, the plurality of sensing regions configured to acquire spatially and temporally resolved measurements of at least one of temperature, pressure, or tissue characterization along one or more measurement paths within or adjacent to the target tissue region.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The embodiments described above are illustrative examples and it should not be construed that the present invention is limited to these particular embodiments. It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the methods or processes). In addition, while certain features of embodiments hereof are described as being performed by a single module, device, or unit for purposes of clarity, it should be understood that the features and functions described herein may be performed by any combination of units or modules. Thus, various changes and modifications may be affected by one skilled in the art without departing from the spirit or scope of the invention as defined in the appended claims.

1.-15. (canceled)

16. An ablation system, comprising:
an ablation probe;
a sensing system associated with the ablation probe and comprising:
at least one temperature sensor configured to generate temperature-related data; and
at least one acoustic sensor configured to generate acoustic data; and
one or more processors configured to:
determine a temperature profile based on the temperature-related data; and
control at least one of operation, sensing, or positioning of the acoustic sensor based on the temperature profile.

17. The system of claim 16, wherein the sensing system comprises an optical fiber, and wherein:
the at least one temperature sensor comprises a plurality of fiber Bragg grating (FBG) sensors disposed along the optical fiber and configured to generate the temperature-related data; and
the at least one acoustic sensor comprises a fiber-end sensor disposed at a distal portion of the optical fiber and configured to generate the acoustic data.

18. The system of claim 17, wherein the one or more processors are further configured to control positioning of the fiber-end sensor relative to a thermal exposure zone based on the temperature profile.

19. The system of claim 17, wherein the one or more processors are further configured to maintain the fiber-end sensor outside of a region exceeding a predetermined temperature threshold.

20. The system of claim 19, wherein the predetermined temperature threshold corresponds to a thermal exposure zone boundary.

21. The system of claim 17, wherein the one or more processors are further configured to determine a distance between the fiber-end sensor and a thermal exposure zone boundary based on the temperature profile.

22. The system of claim 17, wherein the one or more processors are further configured to adjust a position of the ablation probe to limit thermal exposure of the fiber-end sensor.

23. The system of claim 17, further comprising a wavelength division multiplexing (WDM) module configured to simultaneously interrogate the plurality of FBG sensors and the fiber-end sensor.

24. The system of claim 17, wherein each of the plurality of FBG sensors is each associated with a respective wavelength channel.

25. The system of claim 17, wherein acoustic signal modulation at the fiber-end sensor is encoded in at least one wavelength channel.

26. The system of claim 25, further comprising a plurality of photodetectors configured to detect optical signals associated with the respective wavelength channels.

27. The system of claim 17, further comprising a switch configured to selectively couple the optical fiber to one or more sensors.

28. The system of claim 27, wherein the switch comprises a micro-electro-mechanical system (MEMS) switch.

29. The system of claim 28, wherein one or more processors are further configured to control the switch to selectively interrogate the plurality of FBG sensors and the fiber-end sensor.

30. The system of claim 17, wherein the one or more processors are further configured to correlate acoustic data from the fiber-end sensor with the temperature profile to determine a tissue state.

31. The system of claim 16, wherein the one or more processors are further configured to generate control signals for adjusting energy delivery based on both the temperature profile and acoustic data.

32. The system of claim 17, wherein the one or more processors are further configured to determine a thermal gradient and adjust positioning of the fiber-end sensor based on the thermal gradient.

33. A method of operating an ablation system, comprising:
obtaining temperature-related data from a plurality of FBG sensors disposed along an optical fiber;
determining a temperature profile along an ablation probe based on the temperature-related data;
obtaining acoustic data from a fiber-end sensor disposed at a distal portion of the optical fiber; and
controlling at least one of positioning or operation of the fiber-end sensor based on the temperature profile.

34. An ablation system, comprising:
an ablation probe;
an optical fiber extending along at least a portion of the ablation probe;
a plurality of fiber Bragg grating (FBG) sensors disposed along the optical fiber and configured to generate temperature-related data;
a fiber-end sensor disposed at a distal portion of the optical fiber and configured to sense acoustic signals; and
one or more processors configured to:
determine a temperature profile along the ablation probe based on the temperature-related data from the plurality of FBG sensors, and
control at least one of positioning or operation of the fiber-end sensor based on the temperature profile.

35.-56. (canceled)
